(19) United States
(12) Patent Application Publication
(10) Pub. No.: US 2026/0275375 A1
(43) Pub. Date: Sep. 17, 2026

(54) SYNTHETIC CIRCUITS AND USES THEREOF

(71) Applicant: STRAND THERAPEUTICS INC., Boston, MA (US)

(72) Inventors: Tasuku KITADA, Newton, MA (US); Jaspreet KHURANA, Westborough, MA (US); Wen Allen TSENG, Somerville, MA (US); Justin LETENDRE, Stoughton, MA (US); Rachel EICHMAN, Philadelphia, PA (US); Sarah BENING, Cambridge, MA (US); Aidan SIMPSON, Cambridge, MA (US); Dahyana ARIAS ESCAYOLA, Boston, MA (US)

(73) Assignee: STRAND THERAPEUTICS INC., Boston, MA (US)

(21) Appl. No.: 19/473,530

(22) PCT Filed: Apr. 12, 2024
(86) PCT No.: PCT/US2024/024440

§ 371 Date: Oct. 8, 2025

Related U.S. Application Data
(60) Provisional application No. 63/495,744, filed on Apr. 12, 2023.
(60) Provisional application No. 63/504,684, filed on May 26, 2023.
(60) Provisional application No. 63/515,237, filed on Jul. 24, 2023.

(51) Int. Cl. C12N 15/85; A61K 31/7088; A61P 35/00; C07K 14/705; C12N 9/22; C12N 15/88

(52) U.S. Cl. CPC C12N 15/85; A61K 31/7088; A61P 35/00; C07K 14/7051; C12N 9/224; C12N 15/88; C12N 2310/14; C12N 2310/141; C12N 2310/16; C12N 2310/532; C12N 2840/00

(57) ABSTRACT

The present disclosure relates to a synthetic circuit that can be used to selectively express a payload in a target cell. In some aspects, a synthetic circuit comprises a payload sequence comprising a sensor and a regulator sequence comprising a sensor. Also provided herein are methods of using such synthetic circuits to treat a wide range of disease or disorders.

### CROSS REFERENCE TO RELATED APPLICATIONS

This PCT application claims the priority benefit of U.S. Provisional Application No. 63/495,744, filed Apr. 12, 2023; U.S. Provisional Application No. 63/504,684, filed May 26, 2023; and U.S. Provisional Application No. 63/515,237, filed Jul. 24, 2023, the entire disclosures of each which are incorporated herein by reference for all purposes.

### REFERENCE TO SEQUENCE LISTING SUBMITTED ELECTRONICALLY VIA EFS-WEB

The content of the sequence listing is submitted electronically (Name: 4597_017PC04_SequenceListing_ST26.xml; Size: 11,580 bytes; and Date of Creation: Apr. 11, 2024) and is filed with the application is herein incorporated by reference in its entirety.

### BACKGROUND OF THE DISCLOSURE

mRNA therapeutics hold tremendous potential for the treatment of disease. However, controlling expression of payload proteins from mRNA therapeutics in a tissue-specific manner remains a challenge. Therefore, there is a need in the art to develop synthetic RNA-based genetic circuits that yield tightly controlled desired binary output. The presently claimed invention solves this problem through the application of synthetic genetic circuits, in which designed genetic elements selectively express payloads in certain cell types based on sensing of molecular inputs.

### BRIEF SUMMARY OF THE DISCLOSURE

Provided herein is a synthetic circuit comprising: (a) a first nucleotide sequence encoding a payload (payload sequence) and (b) a second nucleotide sequence encoding a regulator (regulator sequence), wherein the payload sequence comprises a sensor that is capable of specifically recognizing the regulator (type P sensor), wherein the regulator sequence comprises a sensor that is capable of specifically recognizing a marker (type R sensor), and wherein the regulator and the marker recognized by the type R sensor (type R marker) are not the same.

In some aspects, the payload sequence comprises a plurality of the type P sensor. In some aspects, the plurality of the type P sensor comprises two type P sensors, three type P sensors, four type P sensors, five type P sensors, six type P sensors, seven type P sensors, or eight or more type P sensors. In some aspects, each of the type P sensors is the same. In some aspects, one or more of the type P sensors are different.

In some aspects, the payload sequence comprises a spacer sequence (type P spacer). In some aspects, the payload sequence comprises a plurality of type P spacer. In some aspects, each of the type P spacers is the same. In some aspects, one or more of the type P spacers are different. In some aspects, (a) at least one type P spacer is positioned upstream of the type P sensor, (b) at least one type P spacer is positioned downstream of the type P sensor, or (c) both (a) and (b). In some aspects, the synthetic circuit described herein (e.g., described above) comprises at least two type P sensors, wherein at least one type P spacer is positioned between the at least two type P sensors.

Also provided herein is a synthetic circuit comprising: (a) a first nucleotide sequence encoding a payload (payload sequence) and (b) a second nucleotide sequence encoding a regulator (regulator sequence), wherein the payload sequence comprises a first sensor that is capable of specifically recognizing the regulator (first type P sensor) and a second sensor that is capable of specifically recognizing a marker (second type P sensor), wherein the regulator sequence comprises a sensor that is capable of specifically recognizing a marker (type R sensor), and wherein the regulator, the marker recognized by the second type P sensor (second type P marker), and/or the marker recognized by the type R sensor (type R marker) are not the same.

In some aspects, the payload sequence comprises a plurality of the first type P sensor. In some aspects, the plurality of the first type P sensor comprises two first type P sensors, three first type P sensors, four first type P sensors, five first type P sensors, six first type P sensors, seven first type P sensors, eight first type P sensors, nine first type P sensors, ten first type P sensors, eleven first type P sensors, or twelve first type P sensors. In some aspects, each of the first type P sensors is the same. In some aspects, one or more of the first type P sensors are different.

In some aspects, the payload sequence comprises a plurality of the second type P sensor. In some aspects, the plurality of the second type P sensor comprises two second type P sensors, three second type P sensors, four second type P sensors, five second type P sensors, six second type P sensors, seven second type P sensors, eight second type P sensors, nine second type P sensors, ten second type P sensors, eleven second type P sensors, or twelve second type P sensors. In some aspects, each of the second type P sensors is the same. In some aspects, one or more of the second type P sensors are different.

In some aspects, a payload sequence provided herein (e.g., described above) comprises a plurality of type P spacer. In some aspects, each of the type P spacers is the same. In some aspects, one or more of the type P spacers are different. In some aspects, (a) at least one type P spacer is positioned between the first type P sensor and the second type P sensor; (b) at least one type P spacer is positioned upstream of both the first type P sensor and the second type P sensor; (c) at least one type P spacer is positioned downstream of both the first type P sensor and the second type P sensor; or (d) any combination of (a) to (c).

Where a synthetic circuit comprises a plurality of first type P sensors, in some aspects, two or more of the first type P sensors are separated by a type P spacer. In some aspects, each of the first type P sensors are separated by a type P spacer.

Where a synthetic circuit comprises a plurality of second type P sensors, in some aspects, two or more of the second type P sensors are separated by a type P spacer. In some aspects, each of the second type P sensors are separated by a type P spacer.

In some aspects, the type P spacer is between about 1 to about 50 nucleotides in length. In some aspects, the type P spacer is at least about 10 nucleotides in length. In some aspects, the type P spacer is about 10 nucleotides in length, about 20 nucleotides in length, or about 50 nucleotides in length. In some aspects, the type P spacer comprises, consists essentially of, or consists of the sequence tttcctttcccccttccctttttcctttcctttcctttcccccttccctt (SEQ ID NO: 1), tttcctttcccccttccctt (SEQ ID NO: 2), or gcggccgctaaa (SEQ ID NO: 3) or fragments thereof.

For any of the synthetic circuits provided herein (e.g., such as those described above), in some aspects, the regulator sequence comprises a plurality of the type R sensor. In some aspects, the plurality of the type R sensor comprises two type R sensors, three type R sensors, four type R sensors, five type R sensors, six type R sensors, seven type R sensors, or eight or more type R sensors. In some aspects, each of the type R sensors is the same. In some aspects, one or more of the type R sensors are different.

In some aspects, the regulator sequence comprises a spacer sequence (type R spacer). In some aspects, the regulator sequence comprises a plurality of type R spacer. In some aspects, each of the type R spacers is the same. In some aspects, one or more of the type R spacers are different.

Where a synthetic circuit comprises a plurality of the type R sensor, in some aspects, two or more of the type R sensors are separated by a type R spacer. In some aspects, each of the type R sensors are separated by a type R spacer. In some aspects, at least one type R spacer is upstream of at least one type R sensor.

In some aspects, the type R spacer is between about 1 to about 50 nucleotides in length. In some aspects, the type R spacer is at least about 10 nucleotides in length. In some aspects, the type R spacer is about 10 nucleotides in length, about 20 nucleotides in length, or about 50 nucleotides in length. In some aspects, the type R spacer comprises, consists essentially of, or consists of the sequence tttcctttcccccttccctttttcctttcctttcctttcccccttccctt (SEQ ID NO: 1) or a fragment thereof. In some aspects, the type R spacer comprises, consists essentially of, or consists of the sequence tttcctttcccccttccctt (SEQ ID NO: 2). In some aspects, the type R spacer comprises, consists essentially of, or consists of the sequence gcggccgctaaa (SEQ ID NO: 3).

For any of the synthetic circuits provided herein (e.g., described above), in some aspects, the first marker, the second marker, or the first and second markers comprise a microRNA, a protein, a metabolite, or combinations thereof.

In some aspects, the regulator comprises a RNA-binding protein, siRNA, shRNA, pre-miRNA, ribozyme, or combinations thereof. In some aspects, the RNA-binding protein comprises a ribonuclease. In some aspects, the ribonuclease comprises a Cas protein. In some aspects, the Cas protein comprises a Cas6 protein.

For any of the synthetic circuits provided herein (e.g., described above), in some aspects, the payload sequence, the regulator sequence, or both the payload and regulator sequences comprise a linear RNA or a circular RNA. In some aspects, the payload sequence is a self-replicating RNA and the regulator sequence is a non-replicating RNA. In some aspects, the payload sequence is a self-replicating RNA and the regulator sequence is a circular RNA. In some aspects, the payload sequence is a self-replicating RNA and the regulator sequence is a linear non-replicating RNA. In some aspects, the payload sequence is a circular RNA and the regulator sequence is a circular RNA. In some aspects, the payload sequence is a circular RNA and the regulator sequence is a linear non-replicating RNA.

The present disclosure further provides a synthetic circuit comprising: (a) a first nucleotide sequence encoding a payload (payload sequence) and (b) a second nucleotide sequence encoding a regulator (regulator sequence); wherein the payload sequence is a self-replicating RNA and comprises a sensor that is capable of specifically recognizing the regulator (type P sensor); wherein the regulator sequence is a circular RNA and comprises a sensor that is capable of specifically recognizing a marker (type R sensor); and wherein the regulator and the marker recognized by the type R sensor (type R marker) are not the same.

Some aspects of the present disclosure is directed to a synthetic circuit comprising: (a) a first nucleotide sequence encoding a payload (payload sequence) and (b) a second nucleotide sequence encoding a regulator (regulator sequence); wherein the payload sequence is a self-replicating RNA and comprises a first sensor that is capable of specifically recognizing the regulator (first type P sensor) and a second sensor that is capable of specifically recognizing a marker (second type P sensor); wherein the regulator sequence is a circular RNA and comprises a sensor that is capable of specifically recognizing a marker (type R sensor); and wherein the regulator, the marker recognized by the second type P sensor (type P marker), and the marker recognized by the type R sensor (type R marker) are not the same.

Also provided herein is a synthetic circuit comprising: (a) a first nucleotide sequence encoding a payload (payload sequence) and (b) a second nucleotide sequence encoding a regulator (regulator sequence); wherein the payload sequence is a self-replicating RNA comprises a sensor that is capable of specifically recognizing the regulator (type P sensor); wherein the regulator sequence is a non-replicating linear RNA and comprises a sensor that is capable of specifically recognizing a marker (type R sensor); and wherein the regulator and the marker recognized by the type R sensor (type R marker) are not the same.

Provided herein is a synthetic circuit comprising: (a) a first nucleotide sequence encoding a payload (payload sequence) and (b) a second nucleotide sequence encoding a regulator (regulator sequence); wherein the payload sequence is a self-replicating RNA and comprises a first sensor that is capable of specifically recognizing the regulator (first type P sensor) and a second sensor that is capable of specifically recognizing a marker (second type P sensor); wherein the regulator sequence is a non-replicating linear RNA and comprises a sensor that is capable of specifically recognizing a marker (type R sensor); and wherein the regulator, the marker recognized by the second type P sensor (type P marker), and the marker recognized by the type R sensor (type R marker) are not the same.

Provided herein is a synthetic circuit comprising: (a) a first nucleotide sequence encoding a payload (payload sequence) and (b) a second nucleotide sequence encoding a regulator (regulator sequence); wherein the payload sequence is a self-replicating RNA and comprises a first sensor that is capable of specifically recognizing the regulator (first type P sensor) and a second sensor that is capable of specifically recognizing a marker (second type P sensor); wherein the regulator sequence is a circular RNA and comprises a sensor that is capable of specifically recognizing a marker (type R sensor); and wherein the regulator, the marker recognized by the second type P sensor (type P marker), and the marker recognized by the type R sensor (type R marker) are not the same.

Provided herein is a synthetic circuit comprising: (a) a first nucleotide sequence encoding a payload (payload sequence) and (b) a second nucleotide sequence encoding a regulator (regulator sequence); wherein the payload sequence is a self-replicating RNA comprises a sensor that is capable of specifically recognizing the regulator (type P sensor); wherein the regulator sequence is a non-replicating linear RNA and comprises a sensor that is capable of specifically recognizing a marker (type R sensor); and wherein the regulator and the marker recognized by the type R sensor (type R marker) are not the same.

Provided herein is a synthetic circuit comprising: (a) a first nucleotide sequence encoding a payload (payload sequence) and (b) a second nucleotide sequence encoding a regulator (regulator sequence); wherein the payload sequence is a self-replicating RNA and comprises a first sensor that is capable of specifically recognizing the regulator (first type P sensor) and a second sensor that is capable of specifically recognizing a marker (second type P sensor); wherein the regulator sequence is a non-replicating linear RNA and comprises a sensor that is capable of specifically recognizing a marker (type R sensor); and wherein the regulator, the marker recognized by the second type P sensor (type P marker), and the marker recognized by the type R sensor (type R marker) are not the same.

Provided herein is a synthetic circuit comprising: (a) a first nucleotide sequence encoding a payload (payload sequence) and (b) a second nucleotide sequence encoding a regulator (regulator sequence); wherein the payload sequence is a circular RNA and comprises a sensor that is capable of specifically recognizing the regulator (type P sensor); wherein the regulator sequence is a circular RNA and comprises a sensor that is capable of specifically recognizing a marker (type R sensor); and wherein the regulator and the marker recognized by the type R sensor (type R marker) are not the same.

Provided herein is a synthetic circuit comprising: (a) a first nucleotide sequence encoding a payload (payload sequence) and (b) a second nucleotide sequence encoding a regulator (regulator sequence); wherein the payload sequence is a circular RNA and comprises a first sensor that is capable of specifically recognizing the regulator (first type P sensor) and a second sensor that is capable of specifically recognizing a marker (second type P sensor); wherein the regulator sequence is a circular RNA and comprises a sensor that is capable of specifically recognizing a marker (type R sensor); and wherein the regulator, the marker recognized by the second type P sensor (type P marker), and the marker recognized by the type R sensor (type R marker) are not the same.

Provided herein is a synthetic circuit comprising: (a) a first nucleotide sequence encoding a payload (payload sequence) and (b) a second nucleotide sequence encoding a regulator (regulator sequence); wherein the payload sequence is a circular RNA and comprises a sensor that is capable of specifically recognizing the regulator (type P sensor); wherein the regulator sequence is a non-replicating linear RNA and comprises a sensor that is capable of specifically recognizing a marker (type R sensor); and wherein the regulator and the marker recognized by the type R sensor (type R marker) are not the same.

Provided herein is a synthetic circuit comprising: (a) a first nucleotide sequence encoding a payload (payload sequence) and (b) a second nucleotide sequence encoding a regulator (regulator sequence); wherein the payload sequence is a circular RNA and comprises a first sensor that is capable of specifically recognizing the regulator (first type P sensor) and a second sensor that is capable of specifically recognizing a marker (second type P sensor); wherein the regulator sequence is a non-replicating linear RNA and comprises a sensor that is capable of specifically recognizing a marker (type R sensor); and wherein the regulator, the marker recognized by the second type P sensor (type P marker), and the marker recognized by the type R sensor (type R marker) are not the same.

For any of the synthetic circuits provided herein (e.g., described above), in some aspects, (a) the payload sequence comprises a plurality of the first type P sensor, (b) the payload sequence comprises a plurality of the second type P sensor, (c) the regulator sequence comprises a plurality of the type R sensor, or (d) any combination of (a) to (c).

In some aspects, (a) the payload sequence comprises a spacer sequence (type P spacer), (b) the regulator sequence comprises a spacer sequence (type R spacer), or (c) both (a) and (b). In some aspects, (a) the type P spacer is positioned between the first type P sensor and the second type P sensor; (b) the type P spacer is positioned upstream of both the first type P sensor and the second type P sensor; (c) the type P spacer is positioned downstream of both the first type P sensor and the second type P sensor; or (d) any combination of (a) to (c).

In some aspects, the payload sequence comprises the plurality of the first type P sensor, wherein two or more of the first type sensors are separated by a type P spacer. In some aspects, the payload sequence comprises the plurality of the second type P sensor, wherein two or more of the second type sensors are separated by a type P spacer. In some aspects, the regulator sequence comprises the plurality of the type R sensor, wherein two or more of the type R sensors are separated by a type R spacer.

In some aspects, the type P spacer, type R spacer, or both are between about 1 to about 50 nucleotides in length. In some aspects, the type P spacer, type R spacer, or both comprise, consist essentially of, or consist of the sequence tttcctttcccccttccctttttcctttcctttcctttcccccttccctt (SEQ ID NO: 1) or a fragment thereof In some aspects, the type P spacer, type R spacer, or both comprise, consist essentially of, or consist of the sequence tttcctttcccccttccctt (SEQ ID NO: 2). In some aspects, the type P spacer, type R spacer, or both comprise, consist essentially of, or consist of the sequence gcggccgctaaa (SEQ ID NO: 3) or a fragment thereof. In some aspects, the type P spacer, type R spacer, or both comprise, consist essentially of, or consist of the sequence gcggccgctaaa (SEQ ID NO: 3).

In some aspects, the type P marker, type R marker, or both comprise a microRNA, a protein, a metabolite, or combinations thereof. In some aspects, the regulator comprises a RNA-binding protein, siRNA, shRNA, pre-miRNA, ribozyme, or combinations thereof.

In some aspects, the regulatory is a RNA-binding protein, wherein the RNA-binding protein comprises a ribonuclease. In some aspects, the ribonuclease comprises a Cas protein. In some aspects, the Cas protein comprises a Cas6 protein.

Provided herein is a synthetic circuit comprising: (a) a first nucleotide sequence encoding a payload (payload sequence) and (b) a second nucleotide sequence encoding a regulator (regulator sequence), wherein, when the payload sequence and the regulator sequence are present in a target cell, the payload is expressed in the target cell for a first expression and the regulator is expressed in the target cell for a second expression, and wherein the first expression is greater than the second expression.

In some aspects, (a) the payload sequence comprises a first sensor that is capable of specifically recognizing the regulator (first type P sensor) and a second sensor that is capable of specifically recognizing a marker (second type P sensor), (b) the regulator sequence comprises a sensor that is capable of specifically recognizing a marker (type R sensor); and wherein the regulator, the marker recognized by the second type P sensor (type P marker), and the marker recognized by the type R sensor (type R marker) are not the same.

In some aspects, the recognition of the type P marker by the second type P sensor inhibits the expression of the payload. In some aspects, the recognition of the type R marker by the type R sensor inhibits the expression of the regulator.

In some aspects, (a) the target cell does not express sufficient levels of the type P marker to turn on the second type P sensor, and (b) the target cell expresses sufficient levels of the type R marker to turn on the type R sensor. In some aspects, (a) the non-target cell expresses sufficient levels of the type P marker to turn on the second type P sensor, (b) the non-target cell does not express sufficient levels of the type R marker to turn on the type R sensor, or (c) both (a) and (b).

Provided herein is a synthetic circuit comprising: (a) a first nucleotide sequence encoding a payload (payload sequence) and (b) a second nucleotide sequence encoding a regulator (regulator sequence), wherein, when the payload sequence and the regulator sequence are present in a non-target cell, the payload is expressed in the non-target cell for a first expression and the regulator is expressed in the non-target cell for a second expression, and wherein the second expression is greater than the first expression.

In some aspects, (a) the payload sequence comprises a first sensor that is capable of specifically recognizing the regulator (first type P sensor) and a second sensor that is capable of specifically recognizing a marker (second type P sensor), and (b) the regulator sequence comprises a sensor that is capable of specifically recognizing a marker (type R sensor); and wherein the regulator, the marker recognized by the second type P sensor (type P marker), and the marker recognized by the type R sensor (type R marker) are not the same.

In some aspects, the binding of the type P marker to the second type P sensor inhibits the expression of the payload. In some aspects, the non-target cell comprises (a) sufficient levels of the type P marker to turn on the second type P sensor, (b) levels of the type R marker insufficient to turn on the type R sensor, or (c) both (a) and (b). In some aspects, the binding of the type R marker to the type R sensor inhibits the expression of the regulator. In some aspects, the target cell comprises: (a) levels of the type P marker insufficient to turn on the second type P sensor, and and (b) sufficient levels of the type R marker to turn on the type R sensor.

Also provided herein is a synthetic circuit comprising: (a) a first nucleotide sequence encoding a payload (payload sequence) and (b) a second nucleotide sequence encoding a regulator (regulator sequence), wherein, when the synthetic circuit is contacted with a population of cells comprising a target cell and a non-target cell, the expression of the payload in the target cell is higher than the corresponding expression in the non-target cell. In some aspects, wherein the expression of the payload in the target cell is higher than the corresponding expression in the non-target cell by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to the corresponding expression in the non-target cell.

In some aspects, (a) the payload sequence comprises a first sensor that is capable of specifically recognizing the regulator (first type P sensor) and a second sensor that is capable of specifically recognizing a marker (second type P sensor), and (b) the regulator sequence comprises a sensor that is capable of specifically recognizing a marker (type R sensor); and wherein the regulator, the marker recognized by the second type P sensor (type P marker), and the marker recognized by the type R sensor (type R marker) are not the same.

In some aspects, the binding of the type P marker to the second type P sensor inhibits the expression of the payload. In some aspects, the non-target cell comprises (a) sufficient levels of the type P marker to turn on the second type P sensor, (b) levels of the type R marker insufficient to turn on the type R sensor, or (c) both (a) and (b). In some aspects, the binding of the type R marker to the type R sensor inhibits the expression of the regulator. In some aspects, the target cell comprises: (a) levels of the type P marker insufficient to turn on the second type P sensor, and and (b) sufficient levels of the type R marker to turn on the type R sensor.

The present disclosure further provides a synthetic circuit comprising: (a) a first nucleotide sequence encoding a payload (payload sequence) and (b) a second nucleotide sequence encoding a regulator (regulator sequence), wherein, when the synthetic circuit is contacted with a population of cells comprising a target cell and a non-target cell, the expression of the payload in the target cell is higher than the corresponding expression in the non-target cell. In some aspects, the expression of the payload in the target cell is higher than the corresponding expression in the non-target cell by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to the corresponding expression in the non-target cell.

In some aspects, (a) the payload sequence comprises a first sensor that is capable of specifically recognizing the regulator (first type P sensor) and a second sensor that is capable of specifically recognizing a marker (second type P sensor), and (b) the regulator sequence comprises a sensor that is capable of specifically recognizing a marker (type R sensor); and wherein the regulator, the marker recognized by the second type P sensor (type P marker), and the marker recognized by the type R sensor (type R marker) are not the same.

In some aspects, the binding of the type P marker to the second type P sensor inhibits the expression of the payload. In some aspects, the binding of the type R marker to the type R sensor inhibits the expression of the regulator. In some aspects, (a) the target cell does not comprise sufficient level of the type P marker to turn on the second type P sensor, and (b) the target cell expresses sufficient levels of the type R marker to turn on the type R sensor. In some aspects, (a) the non-target cell expresses sufficient levels of the type P marker to turn on the second type P sensor, (b) the non-target cell does not express sufficient levels of the type R marker to turn on the type R sensor, or (c) both (a) and (b).

For at least the above-described synthetic circuits, in some aspects, the payload sequence is a self-replicating RNA. In some aspects, the regulator sequence is a non-replicating linear RNA. In some aspects, the payload sequence is a circular RNA. In some aspects, the regulator sequence is a circular RNA. In some aspects, the payload sequence is a self-replicating RNA and the regulator sequence is a circular RNA. In some aspects, the payload sequence is a self-replicating RNA and the regulator sequence is a non-replicating linear RNA. In some aspects, the payload sequence is a circular RNA and the regulator sequence is a circular RNA. In some aspects, the payload sequence is a circular RNA and the regulator sequence is a non-replicating linear RNA.

In some aspects, the above-described synthetic circuits comprise a payload sequence and a regulator sequences, wherein: (a) the payload sequence comprises a plurality of the first type P sensor, (b) the payload sequence comprises a plurality of the second type P sensor, (c) the regulator sequence comprises a plurality of the type R sensor, or (d) any combination of (a) to (c).

In some aspects, (a) the payload sequence comprises a spacer sequence (type P spacer), (b) the payload sequence comprises a spacer sequence (type R spacer), or (c) both (a) and (b). In some aspects, the type P spacer is positioned between the first type P sensor and the second type P sensor. In some aspects, the type P spacer is positioned between the payload coding sequence and (a) the first type P sensor, (b) the second type P sensor, or (c) both (a) and (b). In some aspects, the type R spacer is positioned between the regulator coding sequence and the type R sensor. In some aspects, the payload sequence comprises the plurality of the first type P sensor, wherein two or more of the first type sensors are separated by a type P spacer. In some aspects, the payload sequence comprises the plurality of the second type P sensor, wherein two or more of the second type sensors are separated by a type P spacer. In some aspects, the regulator sequence comprises the plurality of the type R sensor, wherein two or more of the type R sensors are separated by a type R spacer.

In some aspects, the type P spacer, type R spacer, or both are between about 1 to about 50 nucleotides in length. In some aspects, the type P spacer, type R spacer, or both comprise, consist essentially of, or consist of the sequence tttcctttcccccttccctttttcctttcctttcctttcccccttccctt (SEQ ID NO: 1) or a fragment thereof In some aspects, the type P spacer, type R spacer, or both comprise, consist essentially of, or consist of the sequence tttcctttcccccttccctt (SEQ ID NO: 2). In some aspects, the type P spacer, type R spacer, or both comprise, consist essentially of, or consist of the sequence gcggccgctaaa (SEQ ID NO: 3) or a fragment thereof.

In some aspects, the type P marker, type R marker, or both comprise a microRNA. In some aspects, the regulator comprises a RNA-binding protein, siRNA, aptamer, or combinations thereof. In some aspects, the RNA-binding protein comprises a ribonuclease. In some aspects, the ribonuclease comprises a Cas protein. In some aspects, the Cas protein comprises a Cas6 protein.

For any of the synthetic circuits provided in the present disclosure (e.g., described above), in some aspects, the payload comprises a therapeutic protein, reporter protein, immunomodulatory protein, chimeric antigen receptor, or combinations thereof. In some aspects, the payload sequence comprises one or more elements that enhance the translation of the encoded protein as compared to the regulator sequence. In some aspects, the one or more elements comprise an aptamer for a translational initiation factor (e.g., eIF4G).

In some aspects, a synthetic circuit provided herein (e.g., described above) further comprises: (1) an Internal Ribosome Entry Site (IRES), (2) a UTR, (3) a sequence encoding a signal peptide, (4) a translation initiation sequence, (5) a polyA sequence, (6) a sequence encoding a RNA binding protein, (7) a sequence encoding a 2A ribosome skip peptide, or (8) any combination of (1) to (7).

For any of the synthetic circuits provided herein (e.g., described above), in some aspects, the synthetic circuit does not comprise any sequences derived from a non-human genome.

Some aspects of the present disclosure relates to a vector comprising a synthetic circuit provided herein.

Also provided herein are nanoparticles comprising (i) any of the synthetic circuits of the present disclosure (e.g., described above) and (ii) one or more types of lipids and/or lipid like materials. In some aspects, the one or more types of lipid comprise an ionizable lipid, cationic lipid, lipidoid, non-cationic helper lipid, phospholipid, sterol or other structural lipids, or combinations thereof.

In some aspects, the ionizable lipid comprises ((4-hydroxybutyl)azanediyl)bis(hexane-6,1-diyl)bis(2-hexyldecanoate) (ALC-0315), heptadecan-9-yl 8-((2-hydroxyethyl)(6-oxo-6-(undecyloxy)hexyl)amino) octanoate (SM-102), heptadecan-9-yl 8-((2-hydroxyethyl)(8-(nonyloxy)-8-oxooctyl)amino)octanoate (Lipid 5), di((Z)-non-2-en-1-yl) 9-((4-(dimethylamino)butanoyl)oxy)heptadecanedioate (L319), 3-(didodecylamino)-N1,N1,4 tridodecyl-1-piperazineethanamine (KL10), Nl-[2 (didodecylamino)ethyl]-N1,N4,N4-tridodecyl 1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl-4-(dimethylamino)butanoate (DLin-MC3-DMA), 2,2-dilinoleyl-4-(2 dimethylaminoethyl)-[1,3]-dioxolane (DLin-KC2-DMA), 1,2-dioleyloxy-N,N-dimethylaminopropane (DODMA), 2-({8-[(313)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)) or combinations thereof.

In some aspects, the cationic lipid comprises 1,2-dioleoyl-3-trimethylammonium-propane (DOTAP), lipofectamine, N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTEVI), 2,3-dioleyloxy-N-[2(sperminecarboxamido)ethyl]-N,N-dimethyl-l-propanaminium trifluoroacetate (DOSPA), N,N-distearyl-N,N-dimethylammonium bromide (DDAB), N-(1,2-dimyristyloxyprop-3-yl)-N,N-dimethyl-N-hydroxy ethyl ammonium bromide (DMRIE), N-(1,2-dioleoyloxyprop-3-yl)-N,N-dimethyl-N-hydroxy ethyl ammonium bromide (DORIE), N,N-dioleyl-N,N-dimethylammonium chloride (DODAC), 1,2-dilauroyl-sn-glycero-3-ethylphosphocholine (DLePC), 1,2-distearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP), 1,2-distearoyl-sn-glycero-3-ethylphosphocholine (DSePC), 1,2-dipalmitoyl-sn-glycero-3-ethylphosphocholine (DPePC), 1,2-dimyristoyl-sn-glycero-3-ethylphosphocholine (DMePC), 1,2-dioleoyl-sn- glycero-3-ethylphosphocholine (DOePC), 1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), or any combination thereof.

In some aspects, the lipidoid comprises 1,1′-((2-(4-(2-((2-(bis(2-hydroxydodecyl) amino)ethyl) (2-hydroxydodecyl)amino)ethyl) piperazin-1-yl)ethyl)azanediyl) bis(dodecan-2-ol) (C12-200), 3,6-bis(4-(bis(2-hydroxydodecyl)amino)butyl)piperazine2,5-dione (cKK-E12), tetrakis(8-methylnonyl) 3,3′,3″,3′″-(((methylazanediyl) bis(propane-3,1 diyl))bis (azanetriyl))tetrapropionate (306Oi₁₀), G0-C14, 5A2-SC8, 3,6-bis(4-(bis((9Z,12Z)-2-hydroxyoctadeca9,12-dien-1-yl)amino)butyl)piperazine-2,5-dione (OF-02), (((3,6-dioxopiperazine-2,5-diyl)bis(butane-4,1-diyl))bis(azanetriyl))tetrakis(ethane2,1-diyl) (9Z,9′Z,9″Z,9′Z,12Z,12′Z,12″Z,12″Z)-tetrakis (octadeca-9,12-dienoate) (OF-Deg-Lin), (((3,6-dioxopiperazine-2,5-diyl)bis(butane-4,1-diyl)) bis(azanetriyl))tetrakis (butane-4,1-diyl) (9Z,9′Z,9″Z,9′″Z,12Z,12′Z,12″Z,12′″Z)-tetrakis (octadeca-9,12-dienoate) (OF-C4-Deg-Lin), N1,N3,N5-tris(3-(didodecylamino)propyl)benzenel,3,5-tricarboxamide (TT3), Hexa(octan-3-yl) 9,9′,9″,9′″,9″″,9′″″-((((benzene-1,3,5-tricarbonyl)ris(azanediyl)) tris (propane-3,1-diyl))tris(azanetriyl))hexanonanoate (FTT5), PL-1, 98N12-5, ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18 (A2)), A12-Iso5-2DC18 (A12), or any combination thereof. In some aspects, the lipidoid is TT3.

In some aspects, the phospholipid comprises 1,2-dilinoleoyl-sn-glycero-3 phosphocholine (DLPC), 1,2-dimyristoyl-sn-glycerol-phosphocholine (DMPC), 1,2-dioleoyl-sn glycerol-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and any combinations thereof.

In some aspects, the phospholipid is selected from the group consisting of 1-myristoyl-2-palmitoyl-sn-glycero-3-phosphocholine (14:0-16:0 PC, MPPC), 1-myristoyl-2 stearoyl-sn-glycero-3-phosphocholine (14:0-18:0 PC, MSPC), 1-palmitoyl 2-acetyl-sn-glycero-3-phosphocholine (16:0-02:0 PC), 1-palmitoyl-2-myristoyl-sn-glycero-3-phosphocholine (16:0-14:0 PC, PMPC), 1-palmitoyl-2-stearoyl-sn-glycero-3-phosphocholine (16:0-18:0 PC, PSPC), 1-palmitoyl-2-oleoyl-sn-glycero-3-phosphocholine (16:0-18:1 PC, POPC), 1-palmitoyl-2-linoleoyl-sn-glycero-3-phosphocholine (16:0-18:2 PC, PLPC), 1-palmitoyl-2-arachidonoyl-sn-glycero-3-phosphocholine (16:0-20:4 PC), 1-palmitoyl-2-docosahexaenoyl-sn-glycero-3-phosphocholine (14:0-22:6 PC), 1-stearoyl-2-myristoyl-sn-glycero-3-phosphocholine (18:0-14:0 PC, SMPC), 1-stearoyl-2-palmitoyl-sn-glycero-3-phosphocholine (18:0-16:0 PC, SPPC), 1-stearoyl-2-oleoyl-sn-glycero-3-phosphocholine (18:0-18:1 PC, SOPC), 1-stearoyl-2-linoleoyl-sn-glycero-3-phosphocholine (18:0-18:2 PC), 1-stearoyl-2-arachidonoyl-sn-glycero-3-phosphocholine (18:0-20:4 PC), 1-stearoyl-2-docosahexaenoyl-sn-glycero-3-phosphocholine (18:0-22:6 PC), 1-oleoyl-2-myristoyl-sn-glycero-3-phosphocholine (18: 1-14:0 PC, OMPC), 1-oleoyl-2-palmitoyl-sn-glycero-3-phosphocholine (18: 1-16:0 PC, OPPC), 1-oleoyl-2-stearoyl-sn-glycero-3-phosphocholine (18: 1-18:0 PC, OSPC), 1-palmitoyl-2-oleoyl-sn-glycero-3-phosphoethanolamine (16:0-18:1 PE, POPE), 1-palmitoyl-2-linoleoyl-sn-glycero-3-phosphoethanolamine (16:0-18:2 PE), 1-palmitoyl-2-arachidonoyl-sn-glycero-3-phosphoethanolamine (16:0-20:4 PE), 1-palmitoyl-2-docosahexaenoyl-sn-glycero-3-phosphoethanolamine (16:0-22:6 PE), 1-stearoyl-2-oleoyl-sn-glycero-3-phosphoethanolamine (18:0-18:1 PE), 1-stearoyl-2-linoleoyl-sn-glycero-3-phosphoethanolamine (18:0-18:2 PE), 1-stearoyl-2-arachidonoyl-sn-glycero-3-phosphoethanolamine (18:0-20:4 PE), 1-stearoyl-2-docosahexaenoyl-sn-glycero-3-phosphoethanolamine (18:0-22:6 PE), 1-oleoyl-2-cholesterylhemisuccinoyl-sn-glycero-3-phosphocholine (OChemsPC), and any combination thereof.

In some aspects, the sterol comprises a cholesterol, fecosterol, sitosterol, ergosterol, campesterol, stigmasterol, brassicasterol, tomatidine, tomatine, ursolic acid, alpha- tocopherol, and combinations thereof.

In some aspects, the one or more types of lipids and/or lipid like materials are pegylated.

In some aspects, any of the nanoparticles provided herein (e.g., described above), further comprises a targeting ligand.

For any of the nanoparticles provided herein (e.g., described above), in some aspects, the one or more types of lipids and/or lipid like materials comprise a molar ratio of about 10-50% ionizable lipid (e.g., cationic lipid). In some aspects, the one or more types of lipids and/or lipid like materials comprise a molar ratio of about 10-40% phospholipid. In some aspects, the one or more types of lipids and/or lipid like materials comprise a molar ratio of about 20-50% sterol (e.g., cholesterol). In some aspects, the one or more types of lipids and/or lipid like materials comprise a molar ratio of about 0-10% pegylated lipid.

Also provided herein is a pharmaceutical composition comprising any of the synthetic circuits, vectors, or nanoparticles described herein, and a pharmaceutically acceptable carrier. In some aspects, the pharmaceutical composition is formulated for intratumoral, intrathecal, intramuscular, intravenous, subcutaneous, inhalation, intradermal, intralymphatic, intraocular, intraperitoneal, intrapleural, intraspinal, intravascular, nasal, percutaneous, sublingual, submucosal, transdermal, or transmucosal administration.

Also provided herein is a cell comprising any of the synthetic circuits, vectors, or nanoparticles described herein or a cell comprising the payload expressed by the synthetic circuits, vectors, or nanoparticles described herein.

Some aspects of the present disclosure relates to methods of treating a disease or disorder in a subject in need thereof, wherein the method comprises administering to the subject any of the synthetic circuits, vectors, nanoparticles, pharmaceutical compositions, or cells described herein. In some aspects, the subject receives multiple administrations of the synthetic circuit, the vector, the nanoparticle, the pharmaceutical composition, or the cells. In some aspects, the disease or disorder comprises a cancer.

Some aspects of the present disclosure relates to methods of inducing the expression of a payload in a cell, wherein the method comprises contacting the cell with any of the synthetic circuits, vectors, nanoparticles, pharmaceutical compositions, or cells provided herein, wherein the payload is expressed in the cell or on the cell if the regulator is not expressed in the cell.

In some aspects of the methods, the payload comprises a chimeric antigen receptor (CAR), T cell receptor (TCR) or TCR mimic.

In some aspects, the present disclosure provides a method of generating immune cells expressing a chimeric antigen receptor (CAR), T cell receptor (TCR) or TCR mimic in vivo in a subject in need thereof comprising administering the synthetic circuit, the vector, the nanoparticle, or the pharmaceutical composition, wherein the synthetic circuit expresses the CAR, TCR, or TCR mimic as a payload.

In some aspects, the present disclosure provides a method of treating cancer in a subject in need thereof by in situ generated immune cells expressing a chimeric antigen receptor (CAR), T cell receptor (TCR) or TCR mimic, comprising administering the synthetic circuit, the vector, the nanoparticle, or the pharmaceutical composition, wherein the synthetic circuit expresses the CAR, TCR, or TCR mimic as a payload.

In some aspects, the CAR expressed by the synthetic circuit of the present disclosure targets CD19, TRAC, TCRβ, BCMA, CLL-1, CS1, CD38, CD19, TSHR, CD123, CD22, CD30, CD70, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 lRa, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-1 receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTl, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYPIB1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3E, CD4, CD5, CD7, the extracellular portion of the APRIL protein, or any combinations thereof.

In some aspects, the TCR expressed by the synthetic circuit of the present disclosure targets AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-1 receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin BL, MYCN, RhoC, TRP-2, CYPiB1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLECl2A, BST2, EMR2, LY75, GPC3, FCRL5, IGLL1, CD2, CD3F, CD4, CD5, CD7, the extracellular portion of the APRIL protein, or any combinations thereof.

### BRIEF DESCRIPTION OF THE DRAWINGS/FIGURES

FIG. 1 is a schematic of exemplary synthetic circuits described herein. As shown, the regulator sequence and/or the payload sequence can be linear or circular.

FIG. 2 is a schematic showing target site arrays consisting of target sites (TS) that all bind to the same siRNA. Each of the sequences comprises a coding region (encoding mVenus-PEST) and a 3′-UTR. As shown, the sequences include no target site (“No TS”), a single target site (“1X TS”), two target sites (“2X TS”), three target sites (“3X TS”), or four target sites (“4X TS”). As also shown, some of the sequences additionally comprise one or more spacer sequences ranging in length from 10 nucleotides to 50 nucleotides.

FIG. 3 shows the effect of the number of adjacent target sites (TS) immediately following the stop codon of an mVenus-PEST reporter on payload expression. Specifically, the figure provides a graph depicting mVenus median fluorescence in arbitrary units (a.u.) for constructs containing either 1X siRNA TS, 2X siRNA TS, 3X siRNA TS, 4X siRNA TS, no TS, or no reporter following administration of siRNA (0, 1, 10, or 100 nM). Data represent the median collected via flow cytometry from three technical replicates (n=3) at 20 hours post electroporation of reporter and siRNA.

FIG. 4 shows the effect of spacer sequences on payload expression. Specifically, the figure provides a graph depicting mVenus median fluorescence (a.u.) for reporter constructs containing two siRNA target sites that are either immediately adjacent (2X siRNA2), separated by a 20 nucleotide (nt) spacer sequence (2X siRNA2-20nt), or separated by a 50nt spacer sequence (2X siRNA2-50nt), as well as a control reporter construct with no target sites (No TS), and a control condition not transfected with mVenus reporter (No Reporter), following administration of siRNA (0, 1, 10, or 100 nM). Data represent the median fluorescence intensity from three technical replicates (n=3) as determined by flow cytometry at 20 hours post electroporation of reporter and siRNA.

FIG. 5 shows the effect on payload expression of increasing target site copy number when a 20nt spacer sequence is included between target sites. Specifically, the figure provides a graph depicting mVenus median fluorescence (a.u.) for constructs containing two, three, or four siRNA target sites (TS) with a 20 nucleotide (nt) spacer sequence in between neighboring TS (2X siRNA2-20 nt, 3X siRNA2-20 nt, 4X siRNA2-20 nt, respectively), no TS, or no reporter following administration of siRNA (0, 1, 10, or 100 nM). Data represent the median collected via flow cytometry from three technical replicates (n=3) at 20 hours post electroporation of reporter and siRNA.

FIGS. 6A-6B show the assessment of detargeting payload expression for linear (FIG. 6A) and circular (FIG. 6B) RNA. FIG. 6A is a graph depicting mVenus-PEST fluorescence (a.u.) for linear modRNA circuits containing an mVenus-PEST reporter and zero to four miR-b target sites in the 3′ UTR (No miR TS, 1X TS, 2X TS, or 4X TS) in hepatocytes (Huh-7) vs. a control (HEK293T) cell line. Background autofluorescence levels for each cell line are also plotted. Data points represent the average of the geometric mean fluorescence intensity collected from three technical replicates (n=3) at 20 hours post electroporation. FIG. 6B is a graph depicting median fluorescence intensity (a.u.) for circRNA constructs containing 1-4 miR-a target sites immediately following the stop codon of an mVenus-PEST reporter (No TS, 1X TS, 2X TS, or 4X TS) in the HEK293T cell line versus the HeLa cell lines. Data represent the average of the geometric mean fluorescence intensity collected from three technical replicates (n=3) at 4 and 24 hours post electroporation.

FIG. 7A is a schematic showing the position of regulator target sites in an exemplary circRNA expressing mVenus-PEST containing a containing CVB3 IRES, mVenus-PEST, and a regulator (Cas6e) target site in one of five positions. FIG. 7B is a bar graph depicting the effect of the presence or absence of the Cas6e regulator on percent of normalized (no) TS expression in the circRNA construct shown in FIG. 7A compared to electroporation only and a circRNA containing no Cas6e target site knockdown.

FIG. 8A is a schematic showing Nx base pair spacers between the stop codon and array of 3′ UTR target sites. FIGS. 8B-8C are graphs depicting mVenus fluorescence (a.u.) for constructs containing either IX or 2X siRNA Target Sites (TS), and either 0, 7, or 12 nt spacer following administration of siRNA (0, 1, 10, 100 nM). Data represent the geometric mean fluorescence intensity collected via flow cytometry from three technical replicates (n=3) at 6 and 24 hr hours post electroporation of reporter and siRNA.

FIG. 9 is a schematic showing target site arrays for multiple input classifiers.

FIG. 10 is a graph depicting the effect of co-electroporation of siRNA(s) that have or do not have target sites in the target site array on mVenus median fluorescence (a.u.) in HEK293T cells electroporated with an mVenus-PEST reporter containing a target site array immediately following the stop codon and either, no siRNA, siRNA 1, siRNA 2, or both (3X siRNA1-20nt, 3X siRNA2-20nt, 3X siRNA2-siRNA1 Interleaved, 3X siRNA1-siRNA2 Interleaved, 3X siRNA2 3X siRNA1 Adjacent, 3X siRNA1 3X siRNA2 Adjacent, no Target Sites, No Reporter).

FIG. 11 is a schematic showing Nx miR and regulator target sites placed in either the 5′ or 3′ UTRs in locations A, B, and C.

FIG. 12 is a bar graph depicting expression (fold change vs. No miR target sequence (TS)) of circRNA in HEK293T, Huh-7, and HeLa cells electroporated with circRNA 4- and 24 hours post-transfection. Circular RNAs contained either 4x miR-b TS or 4x miR-a TS.

FIGS. 13A-13C show downregulation of circRNA by the regulator Cas6e. FIG. 13A is a schematic showing linear and circular regulator RNA downregulating target circRNA containing a Cas6e target site (TS) and encoding the fluorescent protein mVenus-PEST. FIG. 13B is a bar graph showing that in BHK-21 cells transfected with target RNA containing the Cas6e TS, circRNA and mRNA regulators mVenus expression is reduced to background levels. FIG. 13C is a bar graph showing that Cas6e has no effect on mCherry expression of RNA (mRNA and circRNA) that does not contain its target site.

FIGS. 14A-14B are bar graphs that show the effects of a regulator on linear non-replicating (non-rep) payload mRNA strands bearing a target sequence for the RNA regulator (FIG. 14A) and replicon RNA bearing the RNA regulator's target sequence (FIG. 14B) mRNA. FIG. 14A shows the effect on payload expression (a.u.) for the unmodified RNA (unmodRNA) payload or the modified RNA (modRNA) payload for mRNAs transfected into BHK-21 cells with or without co-transfection of modRNA expressing the RNA regulator. FIG. 14B shows the effect of cells with or without co-transfection of modRNA expressing the RNA regulator on mVenus positive cells (%) for replicon RNA bearing the RNA regulator's target sequence which was transfected at two different doses (20ng or 40ng) into BHK-21 cells.

FIG. 15 is a bar graph depicting expression (fold change vs. No miR target sequence (TS)) of RNA encoding EGFP-PEST driven by the CVB3 IRES in HEK293T and Huh-7 cells electroporated with circular RNA. Circular RNAs contained either no miR TS, 4x miR-b Target Sites, or 4x miR-a Target Sites immediately following the Stop Codon. A No Report Control was also used. The EP Only group (No Reporter Control) was normalized to average of CVB3 without miR TS.

FIG. 16 is a bar graph depicting the ratio of the normalized expression levels of mVenus-PEST expression for HEK293T (non-cancer) cells compared to HeLa (cancer) cells post-electroporation with various human miRNA target sites, corresponding to miRNAs that have higher activity in HEK293T than HeLa cells. The geometric mean of mVenus-PEST expression in each cell type was normalized to that of a modRNA with no miRNA sensors, following subtraction of background fluorescence levels. Flow cytometry data was collected for both cell types (n=3) approximately 24 hours post-electroporation.

FIG. 17A shows the average geometric mean (GMean) of mVenus fluorescence (a.u.) for non-replicating modRNA transfected via electroporation into HEK293T cells or into the human liver cell line Huh-7, and FIG. 17B shows expression of mVenus fluorescence for replicon RNA transfected via electroporation into HEK293T cells or into the human liver cell line Huh-7. A non-replicating modRNA expressing the near-infrared fluorescent reporter protein miRFP720 was co-transfected with each replicon RNA to serve as a transfection marker. Data represents the average geometric mean of three technical replicates (n=3) of flow cytometry data collected approximately 24 hours post-electroporation. The expression output (FIG. 17A) and percentages of miRFP720-positive cells that were also positive for mVenus-PEST (FIG. 17B) were calculated.

FIG. 18 is a bar graph depicting average radiance (p/s/cm2/sr) of firefly luciferase from spleen, lung, kidney, lymph nodes (LN), and liver from mice injected with lipid nanoparticles bearing reporter modRNA encoding firefly luciferase with (grey bars) and without (white bars) the addition of a sensor for the liver-specific microRNA miR-b, or with a vehicle control (black bars). Mice were sacrificed after 6 hours.

FIG. 19 is a bar graph depicting average radiance (p/s/cm2/sr) of firefly luciferase from liver and spleen from mice injected with lipid nanoparticles bearing reporter modRNA encoding firefly luciferase with (grey bars) and without (white bars) the addition of a sensor for a spleen-associated miRNA, miR-h, or with a vehicle control. Mice were sacrificed after 6 hours.

FIG. 20 is a bar graph depicting expression of a green fluorescent reporter (Green Object Mean Intensity, GCU) for Huh7 (black bars) and HEK293T (grey bars) cells transfected with with an RNA circuit consisting of (1) a replicon payload strand expressing a green fluorescent reporter and comprising a first type P sensor responsive to a regulator protein, Cas6e, and (2) a linear non-replicating regulator strand expressing the regulator protein Cas6e and comprising a type R sensor responsive to miR-b. Control Huh7 and HEK293T cells were transfected in parallel with an otherwise identical RNA circuit that lacked a type R sensor. Expression of the green fluorescent payload was measured via quantitative imaging 6 hours post-transfection.

FIG. 21 is a bar graph depicting the effect of a type R sensor on expression of an mVenus reporter protein (Payload Reporter) from a replicon payload RNA sequence. A linear non-replicating regulator sequence was constructed that expresses the Cas6e regulator protein linked to an mCherry reporter via a 2A self-cleaving peptide, such that the Cas6e regulator protein and the mCherry reporter (Regulator Reporter) are co-expressed from the same RNA sequence. Another version of the regulator sequence was constructed that also comprises a type R sensor recognizing miR-i. A549 lung cancer cells, which express high levels of miR-i, were transfected with the payload sequence alone, or were co-transfected with the payload sequence and one of the two versions of the regulator sequence (with or without the type R sensor). At the bottom of the graph, a “+” following “Regulator” indicates that the payload sequence was co-transfected with one of the two versions of the regulator sequence. A “+” following “Type R Sensor” at the bottom of the graph indicates that the payload sequence was co-transfected with the version of the regulator comprising the type R sensor recognizing miR-i. The mean fluorescence intensities (MFI, a.u.) of the mCherry Regulator Reporter and mVenus Payload Reporter in A549 lung cancer cells were measured by flow cytometry.

FIG. 22 shows the effectiveness of the following effectors TTP, cNOT7, and MCPIP1_{PIN}, at inhibiting expression of a modRNA payload (i.e., mVenus, a fluorescent reporter) 0-30 hours after cells were electroporated with the mRNA circuitry. The payload sequence contained 8x PUF_{UGG} TS (i.e., PUF TS #1). An EP only control and a no regulator control were also used. mVenus Object Average Intensity, reflecting the amount of payload expressed, was measured for each group (PUF_{UGG}-TTP, PUF_{UGG}-CNOT7, PUF_{UGG}-MCPIP1_{PIN}, EP control, and no regulator).

FIG. 23 shows the effectiveness of the following effectors TTP, cNOT7, MCPIP1PIN, and DDX6 at inhibiting expression of a repRNA payload (i.e., mVenus, a fluorescent reporter) 0-200 hours after cells were electroporated with the mRNA circuitry. The payload sequence contained 8x PUF_{UGG} TS (i.e., PUF TS #1). An EP only control and a no regulator control were also used. mVenus Mean Intensity Object Average, reflecting the amount of payload expressed, was measured for each group (PUF_{UGG}-TTP, PUF_{UGG}-CNOT7, PUF_{UGG}-MCPIP1PIN, PUFtUGG-DDX6, no regulator, and EP control).

### DETAILED DESCRIPTION OF THE DISCLOSURE

The present disclosure is generally directed to programmable synthetic circuits that can be used to selectively regulate the expression of a gene in a target cell. As further described herein, the synthetic circuits useful for the present disclosure comprise a first nucleotide sequence encoding a payload (payload sequence) and a second nucleotide sequence encoding a regulator (regulator sequence), wherein both the payload sequence and the regulator sequence comprise one or more “sensors,” i.e., target sites capable of recognizing and interacting with other molecules. For example, in some aspects, the payload sequence comprises a sensor that is capable of recognizing a regulator (e.g., the regulator encoded by the regulator sequence) (also referred to herein as a “first type P sensor”), and the regulator sequence comprises a sensor that is capable of recognizing a marker (e.g., a miRNA expressed in a target cell) (also referred to herein as a “type R sensor”). In some aspects, the payload sequence can further comprise an additional type P sensor, which is capable of recognizing a marker (e.g., a miRNA expressed in a host cell) (also referred to herein as a “second type P sensor”). As further described herein, the type P and type R sensors can be activated through recognition of their cognate regulator or marker, and thereby regulate the activity of the payload and regulator sequences (e.g., inhibiting the expression of the encoded protein). Not to be bound by any one theory, through the use of such sensors, which can be specifically programmed based on the markers present in target and/or non-target cells, the synthetic circuits described herein allow for highly specific gene regulation and rapid decision making. Additional aspects of the present disclosure are provided throughout the present application.

### Definitions

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular.

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim.

The term “about” is used herein to mean approximately, roughly, around, or in the regions of When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower), unless indicated otherwise. Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and can vary depending upon the desired properties sought to be obtained. At the very least, and not as an attempt to limit the application of the doctrine of equivalents to the scope of the claims, each numerical parameter should be construed in light of the number of significant digits and ordinary rounding approaches.

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

“Nucleic acid,” “nucleic acid molecule,” “nucleotide sequence,” “nucleic acid sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein. As described herein, a polynucleotide of the present disclosure comprises DNA, RNA, or both. In some aspects, the term “polynucleotide” includes polydeoxyribonucleotides (containing 2-deoxy-D-ribose), polyribonucleotides (containing D-ribose), including tRNA, rRNA, shRNA, siRNA, miRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

As used herein, the term “polypeptide” encompasses both peptides and proteins, unless indicated otherwise.

The term “coding region” refers to a DNA or RNA region (the transcribed region) which “encodes” a particular protein, e.g., such as a payload and/or regulator.

The term “RNA” is used herein to mean a molecule which comprises at least one ribonucleotide residue. “Ribonucleotide” relates to a nucleotide with a hydroxyl group at the 2′-position of a β-D-ribofuranosyl group. The term comprises double-stranded RNA, single-stranded RNA, isolated RNA such as partially or completely purified RNA, essentially pure RNA, synthetic RNA, recombinantly generated RNA such as modified RNA which differs from naturally occurring RNA by addition, deletion, substitution and/or alteration of one or more nucleotides. The term “mRNA” means “messenger-RNA” and relates to a “transcript” which is generated by using a DNA template and encodes a peptide or protein. Typically, a mRNA comprises a 5′-UTR, a protein coding region and a 3′-UTR. mRNA only possesses limited half-life in cells and in vitro. In the context of the present disclosure, mRNA can be generated by in vitro transcription from a DNA template. The in vitro transcription methodology is known to the skilled person. For example, there is a variety of in vitro transcription kits commercially available. As further described herein, in some aspects, a RNA is a linear RNA. In some aspects, a RNA is a circular RNA. In some aspects, a RNA is a self-replicating RNA. In some aspects, a RNA is a non-replicating RNA.

As used herein, the term “genetic circuit” refers to a controllable gene expression system. As described herein, a genetic circuit useful for the present disclosure comprises a synthetic genetic circuit (“synthetic circuit”). As used herein, the term “synthetic circuit” refers to an engineered, non-natural genetic circuit. As is apparent from the present disclosure, synthetic circuits described herein have been specifically programmed to selectively express a payload in a cell of interest (i.e., target cell).

As used herein, the term “self-replicating RNA” refers to a RNA (e.g., mRNA) that is capable of directing its own amplification or replication within a cell (also referred to herein as “repRNA”). To direct its own amplification, the RNA molecule should encode the enzyme(s) necessary to catalyze RNA amplification (e.g., alphavirus nonstructural proteins nsP1, nsP2, nsP3, nsP4) and also contain cis RNA sequences required for replication which are recognized and utilized by the encoded enzymes(s). An alphavirus RNA vector replicon should contain the following ordered elements: 5′ viral or cellular sequences required for nonstructural protein-mediated amplification (may also be referred to as 5′CSE, or 5′ cis replication sequence, or 5′ viral sequences required in cis for replication, or 5′ sequence which is capable of initiating transcription of an alphavirus), sequences which, when expressed, code for biologically active alphavirus nonstructural proteins (e.g., nsP1, nsP2, nsP3, nsP4), and 3′ viral or cellular sequences required for nonstructural protein-mediated amplification (may also be referred as 3′CSE, or 3′ viral sequences required in cis for replication, or an alphavirus RNA polymerase recognition sequence). The alphavirus RNA vector replicon may contain a means to express one or more heterologous sequence(s), such as for example, an IRES or a viral (e.g., alphaviral) subgenomic promoter (e.g., junction region promoter) which may, in certain aspects, be modified in order to increase or reduce viral transcription of the subgenomic fragment, or to decrease homology with defective helper or structural protein expression cassettes, and one or more heterologous sequence(s) to be expressed. A replicon can also contain additional sequences, for example, one or more heterologous sequence(s) encoding one or more polypeptides (e.g., a protein-encoding gene or a 3′ proximal gene) and/or a polyadenylate tract. The replicon should not contain sequences encoding all of the alphavirus structural proteins (capsid, E1, E2). Non-limiting examples of heterologous sequences that can be expressed by replicon vectors are described, for example in U.S. Pat. No. 6,015,686, incorporated by reference in its entirety herein, and include, for example, antigens, lymphokines, cytokines, etc.

As used herein, the term “circular RNA” refers to a RNA (e.g., mRNA) that forms a circular structure through covalent bonds. In the context of the present disclosure, circular RNA can be generated by methodology known to the skilled person (e.g., Wesselhoeft, R. A. et al., 2018, Nature communications, 2018, 9(1), 1-10, herein incorporated by reference in its entirety). As is apparent from the present disclosure, any of the payload sequence and/or regulator sequence can be in the form of a circular RNA. Accordingly, in some aspects, a synthetic circuit provided herein comprises a payload sequence that is a circular RNA. In some aspects, a synthetic circuit provided herein comprises a regulator sequence that is a circular RNA. In some aspects, a synthetic circuit provided herein comprises a payload sequence and a regulator sequence, wherein the payload sequence is a circular RNA and the regulator sequence is a circular RNA. Unless indicated otherwise, a circular RNA is not self-replicating.

As used herein, the term “payload sequence” refers to a nucleotide sequence encoding a payload. As used herein, the term “payload” refers to any protein that can be encoded by the payload sequence. In some aspects, a payload comprises a therapeutic protein. As described herein, unless indicated otherwise, a payload does not comprise a regulator. Non-limiting examples of payloads are provided elsewhere in the present disclosure.

As used herein, the term “regulator sequence” refers to a nucleotide sequence encoding a regulator. As used herein, the term “regulator” comprises any agent that is capable of regulating the expression of the payload encoded by the payload sequence. Non-limiting examples of such regulators are provided elsewhere in the present disclosure. And, as described herein, a regulator useful for the present disclosure is capable of being specifically recognized by a type P sensor on the payload sequence. As also described herein, in some aspects, when the regulator is specifically recognized by a type P sensor, the expression of the payload (encoded by the payload sequence) is reduced or inhibited.

As used herein, the term “sensor” refers to any moiety that is capable of recognizing a marker and/or regulator described herein. As used herein, “recognizing” a marker (or regulator) can comprise the physical interaction between the marker (or the regulator) and the sensor (e.g., the marker binds to a specific marker recognition site within the sensor).

As used herein, the term “type P sensor” refers to a sensor that is present on the payload sequence. Accordingly, a payload sequence useful for the present disclosure comprises a coding region encoding a payload (“payload coding region”) and a type P sensor. In some aspects, a payload sequence can comprise multiple type P sensors. As further described herein, in some aspects, a payload sequence can comprise: (a) payload coding region, (b) a first type P sensor that is capable of recognizing a regulator, and (c) a second type P sensor that is capable of recognizing a marker (“type P marker”). Unless indicated otherwise, the recognition of the type P marker by the second type P sensor activates the second type P sensor, such that the expression of the encoded payload is reduced or inhibited.

As used herein, the term “type R sensor” refers to a sensor that is present on the regulator sequence. In some aspects, a regulator sequence that can be used in constructing a synthetic circuit described herein comprises: (a) a coding region encoding a regulator (“regulator coding region”) and a type R sensor, wherein the type R sensor is capable of recognizing a marker (“type R marker”). Unless indicated otherwise, the recognition of the type R marker by the type R sensor activates the type R sensor, such that the expression of the encoded regulator is reduced or inhibited.

The term “sufficient level” when used to describe a marker (e.g., type P marker and/or type R marker) refers to the amount of the marker required to be recognized by a sensor (e.g., type P sensor and/or type R sensor) and mediate downregulation of the sequence that comprises the sensor. As is apparent from the present disclosure, downregulation of the sequence can result in reduced or inhibition of the expression of any protein encoded by the sequence (e.g., payload and/or regulator). Accordingly, in some aspects, a cell can express the marker and yet the sensor specific to the marker can remain inactive, where the cell does not express sufficient level of the marker.

The term “sequence identity” is used herein to mean a relationship between two or more amino acid (polypeptide or protein) sequences or two or more nucleic acid (polynucleotide) sequences, as determined by comparing the sequences. In certain aspects, sequence identity is calculated based on the full length of two given SEQ ID NO or on part thereof. Part thereof can mean at least 10%, 20%, 30%, 40%, 50%, 60%, 70%, 80%, 90%, or 100% of both SEQ ID NO, or any other specified percentage. The term “identity” can also mean the degree of sequence relatedness between amino acid or nucleic acid sequences, as the case can be, as determined by the match between strings of such sequences.

In some aspects, methods to determine identity are designed to give the largest match between the sequences tested. Methods to determine identity and similarity are codified in publicly available computer programs.

As used herein, the terms “effective amount” or “therapeutically effective amount” of, e.g., a synthetic circuit disclosed herein, refers to a quantity sufficient to, when administered to the subject, including a human, effect beneficial or desired results, including clinical results, and, as such, an “effective amount” or synonym thereto depends on the context in which it is being applied.

As used herein, the term “target cell” refers to a cell in which a payload (e.g., encoded by the payload sequence) is desired to be expressed. As used herein, the term “non-target cell” refers to a cell in which a payload is not intended to be expressed.

### Synthetic Circuits

Provided herein is a synthetic circuit comprising a plurality of nucleotide sequences (e.g., a first nucleotide sequence and a second nucleotide sequence), wherein one or more of the plurality of nucleotide sequences comprises a sensor, which is capable of regulating the activity and/or expression of one or more of the plurality of nucleotide sequences. Accordingly, in some aspects, the present disclosure relates to a polynucleotide (e.g., isolated polynucleotide) comprising: (a) a nucleotide sequence and (b) at least one sensor, wherein the at least one sensor regulates the activity and/or expression of the nucleotide sequence. For example, in some aspects, the nucleotide sequence encodes a payload (payload sequence), and the at least one sensor (type P sensor) is capable of recognizing a marker (e.g., type P marker), wherein when the sensor recognizes the marker, the sensor is activated and thereby regulates the expression of the encoded protein (e.g., payload). In some aspects, the nucleotide sequence comprises a regulator (regulator sequence), and the at least one sensor (type R sensor) is capable of recognizing a marker (e.g., type R marker), wherein when the sensor recognizes the marker, the sensor is activated and thereby, regulates the expression of the regulator. As used herein, regulating the “expression of the regulator” can comprise: (i) regulating the amount of regulator expressed in the cell, (ii) regulating the activity of the regulator, or (iii) both (i) and (ii). Similarly, regulating the “expression of the payload” can comprise: (i) regulating the amount of payload expressed in the cell, (ii) regulating the activity of the payload, or (iii) both (i) and (ii).

In some aspects, provided herein is a synthetic circuit comprising: (a) a first nucleotide sequence encoding a payload (payload sequence) and (b) a second nucleotide sequence encoding a regulator (regulator sequence); wherein the payload sequence comprises a sensor (type P sensor) that is capable of recognizing the regulator; and wherein the regulator sequence comprises a sensor (type R sensor) that is capable of recognizing a marker expressed in a cell. In some aspects, the recognition of the marker by the type R sensor reduces or inhibits the expression of the regulator. Inhibiting expression of the regulator thereby prevents activation of the type P sensor; expression of the encoded payload is thereby permitted.

### Payload Sequence

As is apparent from the present disclosure, in some aspects, a synthetic circuit comprises a nucleotide sequence encoding a payload (payload sequence). In some aspects, the payload sequence can encode any suitable proteins known in the art. Non-limiting examples of suitable payloads include a therapeutic protein, reporter protein, immunomodulatory protein, chimeric antigen receptor (CAR), or combinations thereof.

In some aspects, the payload sequence is linear (e.g., linear RNA). In some aspects, the payload sequence is circular (e.g., circular RNA). In some aspects, the payload sequence is self-replicating (e.g., self-replicating RNA). In some aspects, the payload sequence is non-replicating (e.g., non-replicating RNA).

### Type P Sensors

In some aspects, the payload sequence comprises a sensor that is capable of recognizing a regulator (e.g., encoded by the regulator sequence). In some aspects, the payload sequence comprises a sensor that is capable of recognizing a marker. In some aspects, the payload sequence comprises a first sensor that is capable of recognizing the regulator (first type P sensor) and a second sensor that is capable of recognizing a marker (second type P sensor).

Accordingly, in some aspects, provided herein is a synthetic circuit comprising: (a) a first nucleotide sequence encoding a payload (payload sequence) and (b) a second nucleotide sequence encoding a regulator (regulator sequence), wherein the payload sequence comprises a first sensor that is capable of recognizing the regulator (first type P sensor) and a second sensor that is capable of recognizing a marker (second type P sensor). In some aspects, the first type P sensor and the second type P sensor are different (i.e., does not recognize the same ligand). In some aspects, a synthetic circuit provided herein comprises a plurality of sensors. For instance, in some aspects, a payload sequence provided herein comprises a plurality of type P sensors. In some aspects, each of the plurality of sensors on the payload sequence is the same. In some aspects, one or more of the plurality of sensors on the payload sequence are different. Where the payload sequence comprises a first type P sensor (e.g., recognizing a regulator) and a second type P sensor (e.g., recognizing a marker), in some aspects, the payload sequence comprises a plurality of first type P sensors. For example in some aspects, the payload sequence comprises about two first type P sensors, about three first type P sensors, about four first type P sensors, about five first type P sensors, about six first type P sensors, about seven first type P sensors, about eight first type P sensors, about nine first type sensors, about 10 first type P sensors, about 11 first type P sensors, about 12 first type P sensors, about 13 first type P sensors, about 14 first type P sensors, about 15 first type P sensors, about 16 first type P sensors, about 17 first type P sensors, about 18 first type P sensors, about 19 first type P sensors, or about 20 or more first type P sensors. In some aspects, the payload sequence comprises at least two first type P sensors. In some aspects, the payload sequence comprises at least three first type P sensors. In some aspects, the payload sequence comprises at least four first type P sensors. In some aspects, the payload sequence comprises at least five first type P sensors. In some aspects, the payload sequence comprises at least six first type P sensors. In some aspects, the payload sequence comprises at least seven first type P sensors. In some aspects, the payload sequence comprises at least eight first type P sensors. In some aspects, the payload sequence comprises at least nine first type P sensors. In some aspects, the payload sequence comprises at least 10 first type P sensors. In some aspects, the payload sequence comprises at least 11 first type P sensors. In some aspects, the payload sequence comprises at least 13 first type P sensors. In some aspects, the payload sequence comprises at least 13 first type P sensors. In some aspects, the payload sequence comprises at least 14 first type P sensors. In some aspects, the payload sequence comprises at least 15 first type P sensors. In some aspects, the payload sequence comprises at least 16 first type P sensors. In some aspects, the payload sequence comprises at least 17 first type P sensors. In some aspects, the payload sequence comprises at least 18 first type P sensors. In some aspects, the payload sequence comprises at least 19 first type P sensors. In some aspects, the payload sequence comprises at least 20 first type P sensors.

In some aspects, each of the first type P sensors is the same. For instance, in some aspects, a synthetic circuit described herein comprises a payload sequence and a regulator sequence, wherein the payload sequence comprises a plurality of the first type P sensor, and wherein each of the plurality of the first type P sensor specifically recognizes the same regulator (e.g., each of the first type P sensor comprises the same binding site for the regulator). In some aspects, one or more of the first type P sensors are different. For instance, in some aspects, one or more of the first type P sensors recognize a different regulator. In some aspects, one or more of the first type P sensors recognize a different binding site on the same regulator.

In some aspects, where the payload sequence comprises a first type P sensor (e.g., recognizing a regulator) and a second type P sensor (e.g., recognizing a marker), the payload sequence comprises a plurality of second type P sensors. For example, in some aspects, the payload sequence comprises about two second type P sensors, about three second type P sensors, about four second type P sensors, about five second type P sensors, about six second type P sensors, about seven second type P sensors, about eight second type P sensors, about nine second type P sensors, about 10 second type P sensors, about 11 second type P sensors, about 12 second type P sensors, about 13 second type P sensors, about 14 second type P sensors, about 15 second type P sensors, about 16 second type P sensors, about 17 second type P sensors, about 18 second type P sensors, about 19 second type P sensors, or about 20 or more second type P sensors. In some aspects, the payload sequence comprises at least two second type P sensors. In some aspects, the payload sequence comprises at least three second type P sensors. In some aspects, the payload sequence comprises at least four second type P sensors. In some aspects, the payload sequence comprises at least five second type P sensors. In some aspects, the payload sequence comprises at least six second type P sensors. In some aspects, the payload sequence comprises at least seven second type P sensors. In some aspects, the payload sequence comprises at least eight second type P sensors. In some aspects, the payload sequence comprises at least nine second type P sensors. In some aspects, the payload sequence comprises at least 10 second type P sensors. In some aspects, the payload sequence comprises at least 11 second type P sensors. In some aspects, the payload sequence comprises at least 12 second type P sensors. In some aspects, the payload sequence comprises at least 13 second type P sensors. In some aspects, the payload sequence comprises at least 14 second type P sensors. In some aspects, the payload sequence comprises at least 15 second type P sensors. In some aspects, the payload sequence comprises at least 16 second type P sensors. In some aspects, the payload sequence comprises at least 17 second type P sensors. In some aspects, the payload sequence comprises at least 18 second type P sensors. In some aspects, the payload sequence comprises at least 19 second type P sensors. In some aspects, the payload sequence comprises at least 20 second type P sensors.

In some aspects, each of the second type P sensors is the same. For instance, in some aspects, a synthetic circuit comprises a payload sequence and a regulator sequence, wherein the payload sequence comprises a plurality of the second type P sensors, and wherein each of the plurality of the second type P sensors specifically recognizes the same marker. In some aspects, one or more of the second type P sensors are different. In some aspects, one or more of the second type P sensors specifically recognizes a different marker. In some aspects, one or more of the second type P sensors recognize a different binding site on the same marker.

In some aspects, where a payload sequence comprises a first type P sensor (e.g., recognizing a regulator) and a second type P sensor (e.g., recognizing a marker), the payload sequence comprises a plurality of first type P sensors and a plurality of second type P sensors. In some aspects, the payload sequence comprises: (a) about two first type P sensors, about three first type P sensors, about four first type P sensors, about five first type P sensors, about six first type P sensors, about seven first type P sensors, about eight first type P sensors, about nine first type sensors, about 10 first type P sensors, about 11 first type P sensors, about 12 first type P sensors, about 13 first type P sensors, about 14 first type P sensors, about 15 first type P sensors, about 16 first type P sensors, about 17 first type P sensors, about 18 first type P sensors, about 19 first type P sensors, or about 20 or more first type P sensors; (b) about two second type P sensors, about three second type P sensors, about four second type P sensors, about five second type P sensors, about six second type P sensors, about seven second type P sensors, about eight second type P sensors, about nine second type P sensors, about 10 second type P sensors, about 11 second type P sensors, about 12 second type P sensors, about 13 second type P sensors, about 14 second type P sensors, about 15 second type P sensors, about 16 second type P sensors, about 17 second type P sensors, about 18 second type P sensors, about 19 second type P sensors, or about 20 or more second type P sensors; or (c) both (a) and (b). As further described herein, in some aspects, each of the first type P sensors is the same. In some aspects, one or more of the first type P sensors are different. In some aspects, each of the second type P sensors is the same. In some aspects, one or more of the second type P sensors are different.

In some aspects, a first type P sensor comprises a target site for PUF RNA binding domain.

In some aspects, the first type P sensor comprises a PUF target site (PUF TS) comprising one or more nucleic acid sequences as set forth in 5′-UGUAUAUA-3′ (PUF WT TS), 5′-UGGAUGAA-3′ (PUF TS #1), 5′-UGUACGUC-3′ (PUF TS #2), 5′-UCUACGUC-3′ (PUF TS #3), 5′-UGUACGAC-3′ (PUF TS #4), 5′-UGUCCGUC-3′ (PUF TS #5), 5′-UGUACGUG-3′ (PUF TS #6), 5′-UGGAAGUC-3′ (PUF TS #7), 5′-UGUGCCUC-3′ (PUF TS #8), or 5′-UGUAGCU A-3′ (PUF TS #9).

In some aspects, the first type P sensor comprises the nucleic acid sequence as set forth in 5′-UGUAUAUA-3′ (PUF WT TS). In some aspects, the first type P sensor comprises the nucleic acid sequence as set forth in 5′-GGAUGAA-3′ (PUF TS #1). In some aspects, the first type P sensor comprises the nucleic acid sequence as set forth in 5′-UGUACGUC-3′ (PUF TS #2). In some aspects, the first type P sensor comprises the nucleic acid sequence as set forth in 5′-UCUACGUC-3′ (PUF TS #3). In some aspects, the first type P sensor comprises the nucleic acid sequence as set forth in 5′-UGUACGAC-3′ (PUF TS #4). In some aspects, the first type P sensor comprises the nucleic acid sequence as set forth in 5′-UGUCCGUC-3′ (PUF TS #5). In some aspects, the first type P sensor comprises the nucleic acid sequence as set forth in 5′-UGUACGUG-3′ (PUF TS #6). In some aspects, the first type P sensor comprises the nucleic acid sequence as set forth in 5′-UGGAAGUC-3′ (PUF TS #7). In some aspects, the first type P sensor comprises the nucleic acid sequence as set forth in 5′-UGUGCCUC-3′ (PUF TS #8). In some aspects, the first type P sensor comprises the nucleic acid sequence as set forth in 5′-UGUAGCU A-3′ (PUF TS #9). In some aspects, the first type P sensor comprises at least 8 nucleotides in length.

### Type P Spacers

In some aspects, a payload sequence useful for the present disclosure further comprises a spacer sequence (“type P spacer”). In some aspects, the payload sequence comprises a plurality of type P spacers. For instance, in some aspects, the payload sequence comprises about two type P spacers, about three type P spacers, about four type P spacers, about five type P spacers, about six type P spacers, about seven type P spacers, about eight type P spacers, about nine type P spacers, or about 10 or more type P spacers. In some aspects, the payload sequence comprises at least two type P spacers. In some aspects, the payload sequence comprises at least three type P spacers. In some aspects, the payload sequence comprises at least four type P spacers. In some aspects, the payload sequence comprises at least five type P spacers. In some aspects, the payload sequence comprises at least six type P spacers. In some aspects, the payload sequence comprises at least seven type P spacers. In some aspects, the payload sequence comprises at least eight type P spacers. In some aspects, the payload sequence comprises at least nine type P spacers. In some aspects, the payload sequence comprises at least 10 type P spacers. In some aspects, each of the type P spacers are the same. In some aspects, one or more of the type P spacers are different.

Accordingly, in some aspects, a synthetic circuit provided herein comprises a payload sequence, wherein the payload sequence comprises a type P sensor (e.g., specifically recognizing a regulator and/or marker) and a type P spacer. In some aspects, the type P spacer is upstream of the type P sensor (e.g., the type P spacer is positioned closer to the 5′-end of the payload sequence as compared to the type P sensor). In some aspects, the type P spacer is downstream of the type P sensor (e.g., the type P spacer is positioned closer to the 3′-end of the payload sequence as compared to the type P sensor). In some aspects, the type P sensor is downstream of the coding region of the payload sequence, and the type P spacer is positioned between the coding region of the payload sequence and the type P sensor (e.g., after the stop codon of the coding region and before the beginning of the type P sensor). As used herein, the term “coding region of the payload sequence” refers to the portion of the payload sequence that specifically encodes for the payload.

Where a payload sequence comprises a plurality of type P sensors, in some aspects, the type P spacer is upstream of one or more of the plurality of type P sensors. In some aspects, the type P spacer is downstream of one or more of the plurality of type P sensor. In some aspects, the type P spacer is positioned in between at least two of the type P sensors. In some aspects, each of the plurality of type P sensors are separated by a type P spacer. For instance, in some aspects, a synthetic circuit provided herein comprises a payload sequence, wherein the payload sequence comprises a first type P sensor (e.g., specifically recognizes a regulator), a second type P sensor (e.g., specifically recognizes a marker), and a type P spacer, wherein the type P spacer is is positioned in between the first type P sensor and the second type P sensor. As described herein, in some aspects, the payload sequence comprises a plurality of first type P sensors, wherein each of the plurality of first type P sensors are separated by a type P spacer. In some aspects, the payload sequence comprises a plurality of second type P sensors, wherein each of the plurality of second type P sensors are separated by a type P spacer. In some aspects, a payload sequence comprises a plurality of first type P sensors and a plurality of second type P sensors, wherein: (a) each of the plurality of first type P sensors are separated by a type P spacer, (b) each of the plurality of second type P sensors are separated by a type P spacer, and (c) both (a) and (b).

Where the payload sequence comprises a plurality of type P spacers, in some aspects, each of the type P spacers are the same. In some aspects, one or more of the type P spacers are different. Not to be bound by any one theory, in some aspects, type P spacers useful for the present disclosure are of suitable lengths such that the spacers aid in the binding of the type P sensors to their ligand (e.g., regulator and/or markers). In some aspects, the type P spacer is between about 1 to about 100 nucleotides in length. In some aspects, the type P spacer is about 1 nucleotide in length, about 5 nucleotides in length, about 10 nucleotides in length, about 15 nucleotides in length, about 20 nucleotides in length, about 25 nucleotides in length, about 30 nucleotides in length, about 35 nucleotides in length, about 40 nucleotides in length, about 45 nucleotides in length, about 50 nucleotides in length, about 55 nucleotides in length, about 60 nucleotides in length, about 65 nucleotides in length, about 70 nucleotides in length, about 75 nucleotides in length, about 80 nucleotides in length, about 85 nucleotides in length, about 90 nucleotides in length, about 95 nucleotides in length, or about 100 nucleotides in length. In some aspects, the type P spacer is between about 1 to about 50 nucleotides length. In some aspects, the type P spacer is about 5 nucleotides in length. In some aspects, the type P spacer is about 10 nucleotides in length. In some aspects, the type P spacer is about 15 nucleotides in length. In some aspects, the type P spacer is about 20 nucleotides in length. In some aspects, the type P spacer is about 25 nucleotides in length, In some aspects, the type P spacer is about 30 nucleotides in length, In some aspects, the type P spacer is about 35 nucleotides in length. In some aspects, the type P spacer is about 40 nucleotides in length. In some aspects, the type P spacer is about 45 nucleotides in length. In some aspects, the type P spacer is about 50 nucleotides in length.

Unless indicated otherwise, type P spacers useful for the present disclosure are not limited to any specific nucleotide sequences, as long as the type P spacers are of sufficient length to carry out their intended function (e.g., aid in the binding of the type P sensors to their ligands). Accordingly, in some aspects, a type P spacer useful for the present disclosure comprises a randomly generated nucleotide sequence. In some aspects, where a plurality of type P spacers are used in separating a plurality of type P sensors, one or more of the plurality of type P spacers have a difference sequence, such that the plurality of type P spacers do not include randomly generated nucleotide sequences that repeat. In some aspects, a type P spacer useful for the present disclosure comprises, consists essentially of, or consists of the sequence tttcctttcccccttccctttttcctttcctttcctttcccccttccctt (SEQ ID NO: 1) or a fragment thereof. In some aspects, the type P spacer comprises the sequence set forth in SEQ ID NO: 1. In some aspects, the type P spacer consists essentially of the sequence set forth in SEQ ID NO: 1. In some aspects, the type P spacer consists of the sequence set forth in SEQ ID NO: 1. In some aspects, a type P spacer useful for the present disclosure comprises, consists essentially of, or consists of the sequence tttcctttcccccttccctt (SEQ ID NO: 2) or a fragment thereof. In some aspects, the type P spacer comprises the sequence set forth in SEQ ID NO: 2. In some aspects, the type P spacer consists essentially of the sequence set forth in SEQ ID NO: 2. In some aspects, the type P spacer consists of the sequence set forth in SEQ ID NO: 2. In some aspects, a type P spacer useful for the present disclosure comprises, consists essentially of, or consists of the sequence gcggccgctaaa (SEQ ID NO: 3) or a fragment thereof. In some aspects, the type P spacer comprises the sequence set forth in SEQ ID NO: 3. In some aspects, the type P spacer consists essentially of the sequence set forth in SEQ ID NO: 3. In some aspects, the type P spacer consists of the sequence set forth in SEQ ID NO: 3. In some aspects, the type P spacer is the same as the type R spacer (further described elsewhere in the present disclosure). In some aspects, the type P spacer is different than the type R spacer.

TABLE 1
Exemplary Type P Spacers

| | |
|---|---|
| SEQ ID NO: 1 | tttcctttcccccttccctttttcctttcctttcc |
| | tttcccccttccctt |
| SEQ ID NO: 2 | tttcctttcccccttccctt |
| SEQ ID NO: 3 | gcggccgctaaa |

### Regulator Sequence

As described herein, in some aspects, a synthetic circuit comprises a nucleotide sequence which comprises or encodes a regulator (“regulator sequence”). Accordingly, in some aspects, a synthetic circuit of the present disclosure comprises a payload sequence (e.g., any of the payload sequences described above) and a regulator sequence. Non-limiting examples of regulators that can be used with the present disclosure are provided elsewhere in the present disclosure. In some aspects, a regulator comprises an RNA binding domain (RBD) of a human Pum1 protein (“PUF RBD”). Therefore in some aspects, a regulator sequence comprises a nucleotide sequence encoding a PUF RBD. In some aspects, PUF RBD comprises the amino acid sequence as set forth in SEQ ID NO:

(GRSRLLEDFRNNRYPNLQLREIAGHIMEFSQDQHGSRFIQLKLERATPA
ERQLVFNEILQAAYQLMVDVFGNYVIQKFFEFGSLEQKLALAERIRGHVL
SLALQMYGCRVIQKALEFIPSDQQNEMVRELDGHVLKCVKDQNGNHVVQK
CIECVQPQSLQFIIDAFKGQVFALSTHPYGCRVIQRILEHCLPDQTLPIL
EELHQHTEQLVQDQYGNYVIQHVLEHGRPEDKSKIVAEIRGNVLVLSQHK
FASNVVEKCVTHASRTERAVLIDEVCTMNDGPHSALYTMMKDQYANYVVQ
KMIDVAEPGQRKIVMHKIRPHIATLRKYTYGKHILAKLEKYYMKNGVDL
G).

In some aspects, the PUF RBD comprises one or more amino acid substitution compared to the wild type PUF RBD as set forth in SEQ ID NO: 6. In some aspects, the PUF RBD comprises an amino acid sequence having at least about 70%, at about 75%, at least about 80%, at least about 85%, at least about 90%, at least about 95%, at least about 96%, at least about 97%, at least about 98%, at least about 99%, or about 100% sequence identity to the wild type PUM-RBD as set forth in SEQ ID NO: 6, wherein the PUF RBD is capable of binding to the PUF TS. In some aspects, the amino acid substitution in the PUF RBD comprises a substitution corresponding to an amino acid position in R1, R2, R3, R4, R5, R6, R7, or R8 of the wild type PUF RBD as set forth in SEQ ID NO: 6, or any combination thereof.

In some aspects, the regulator useful for the present disclosure further comprises an effector domain. In some aspects, the effector domain comprises a degradation domain, a translation inhibition domain, a protein recruiting domain, or any combination thereof. In some aspects, the effector domain comprises a degradation domain.

In some aspects, the effector domain comprises a degradation domain that is derived from cNOT7, TTP, MCPIP1_{PIN}, DDX6, Dis3PIN, SMG6PIN, or any combination thereof.

In some aspects, the effector domain is derived from cNOT7.

In some aspects, the effector domain is derived from TTP.

In some aspects, the effector domain is derived from MCPIP1PIN.

In some aspects, the degradation domain is derived from DDX6.

In some aspects, the degradation domain is derived from Dis3PIN.

In some aspects, the degradation domain is derived from SMG6PIN.

In some aspects, the regulator sequence is linear (e.g., linear RNA). In some aspects, the regulator sequence is circular (e.g., circular RNA). In some aspects, the regulator sequence is non-replicating (e.g., non-replicating RNA).

### Type R Sensors

In some aspects, the regulator sequence comprises a sensor that is capable of specifically recognizing a marker. Accordingly, some aspects of the present disclosure relate to a synthetic circuit comprising a payload sequence and a regulator sequence, wherein the payload sequence comprises a sensor (type P sensor), and wherein the regulator sequence comprises a sensor (“type R sensor”). In some aspects, present disclosure provides a synthetic circuit comprising a payload sequence and a regulator sequence, wherein the payload sequence comprises a first type P sensor (e.g., specifically recognizes a regulator) and a second type P sensor (e.g., specifically recognizes a marker), and wherein the regulator sequence comprises a type R sensor (e.g., specifically recognizes a marker). Where a synthetic circuit described herein comprises both a type P sensor and a type R sensor, in some aspects, the type P sensor and the type R sensor are not the same (e.g., do not specifically recognize the same ligand).

In some aspects, a regulator sequence useful for the present disclosure comprises a plurality of sensors. For example, in some aspects, a regulator sequence comprises about two type R sensors, about three type R sensors, about four type R sensors, about five type R sensors, about six type R sensors, about seven type R sensors, about eight type R sensors, about nine type R sensors, or about 10 or more type R sensors. In some aspects, the regulator sequence comprises at least two type R sensors. In some aspects, the regulator sequence comprises at least three type R sensors. In some aspects, the regulator sequence comprises at least four type R sensors. In some aspects, the regulator sequence comprises at least five type R sensors. In some aspects, the regulator sequence comprises at least six type R sensors. In some aspects, the regulator sequence comprises at least seven type R sensors. In some aspects, the regulator sequence comprises at least eight type R sensors. In some aspects, the regulator sequence comprises at least nine type R sensors. In some aspects, the regulator sequence comprises at least 10 type R sensors.

In some aspects, each of the plurality of sensors on the regulator sequence is the same. For instance, in some aspects, a synthetic circuit described herein comprises a payload sequence and a regulator sequence, wherein the regulator sequence comprises a plurality of type R sensors, and wherein each of the plurality of type R sensors recognize the same marker (e.g., each of the type R sensors comprise the same binding site for the marker). In some aspects, one or more of the plurality of sensors on the regulator sequence are different. For instance, in some aspects, one or more of the plurality of type R sensors recognize different markers. In some aspects, one or more of the plurality of type R sensors recognize a different binding site for the same marker.

### Type R Spacers

In some aspects, a regulator sequence provided herein further comprises a spacer sequence (“type R spacer”). Accordingly, in some aspects, a synthetic circuit provided herein comprises a payload sequence and a regulator sequence, wherein the payload sequence comprises a type P sensor (e.g., first type P sensor and/or second type P sensor) and a type P spacer, and wherein the regulator sequence comprises a type R sensor and a type R spacer. In some aspects, the type R spacer and the type P spacer are not the same. In some aspects, the type R spacer and the type P spacer are the same.

In some aspects, a regulator sequence comprises a plurality of type R spacers. In some aspects, the regulator sequence comprises about two type R spacers, about three type R spacers, about four type R spacers, about five type R spacers, about six type R spacers, about seven type R spacers, about eight type R spacers, about nine type R spacers, or about 10 or more type R spacers. In some aspects, the regulator sequence comprises at least two type R spacers. In some aspects, the regulator sequence comprises at least three type R spacers. In some aspects, the regulator sequence comprises at least four type R spacers. In some aspects, the regulator sequence comprises at least five type R spacers. In some aspects, the regulator sequence comprises at least six type R spacers. In some aspects, the regulator sequence comprises at least seven type R spacers. In some aspects, the regulator sequence comprises at least eight type R spacers. In some aspects, the regulator sequence comprises at least nine type R spacers. In some aspects, the regulator sequence comprises at least 10 type R spacers. In some aspects, each of the type R spacers are the same. In some aspects, one or more of the type R spacers are different.

In some aspects, the type R spacer is positioned upstream of the type R sensor within the regulator sequence. In some aspects, the type R spacer is positioned downstream of the type R sensor. In some aspects, where the regulator sequence encodes the regulator, the type R spacer is positioned between the coding region of the regulator sequence and the type R sensor (e.g., after the stop codon of the coding region and before the beginning of the type R sensor). As used herein, the term “coding region of the regulator sequence” refers to the portion of the regulator sequence that specifically encodes for the regulator.

Where the regulator sequence comprises a plurality of type R sensors, in some aspects, the type R spacer is upstream of one or more of the plurality of type R sensors. In some aspects, the type R spacer is downstream of one or more of the plurality of type R sensors. In some aspects, the type R spacer is positioned in between at least two of the plurality of type R sensors. In some aspects, each of the plurality of type R sensors are separated by a type R spacer.

Where the regulator sequence comprises a plurality of type R spacers, in some aspects, each of the type R spacers are the same. In some aspects, one or more of the type R spacers are different. In some aspects, a type R spacer can be of any suitable lengths such that the type R spacer aids in the binding of the type R sensor to its ligand (e.g., marker). In some aspects, the type R spacer is between about 1 to about 100 nucleotides in length. In some aspects, the type R spacer is about 1 nucleotide in length, about 5 nucleotides in length, about 10 nucleotides in length, about 15 nucleotides in length, about 20 nucleotides in length, about 25 nucleotides in length, about 30 nucleotides in length, about 35 nucleotides in length, about 40 nucleotides in length, about 45 nucleotides in length, about 50 nucleotides in length, about 55 nucleotides in length, about 60 nucleotides in length, about 65 nucleotides in length, about 70 nucleotides in length, about 75 nucleotides in length, about 80 nucleotides in length, about 85 nucleotides in length, about 90 nucleotides in length, about 95 nucleotides in length, or about 100 nucleotides in length. In some aspects, the type R spacer is between about 1 to about 50 nucleotides in length. In some aspects, the type R spacer is about 5 nucleotides in length. In some aspects, the type R spacer is about 10 nucleotides in length. In some aspects, the type R spacer is about 15 nucleotides in length. In some aspects, the type R spacer is about 20 nucleotides in length. In some aspects, the type R spacer is about 25 nucleotides in length. In some aspects, the type R spacer is about 30 nucleotides in length. In some aspects, the type R spacer is about 35 nucleotides in length. In some aspects, the type R spacer is about 40 nucleotides in length. In some aspects, the type R spacer is about 45 nucleotides in length. In some aspects, the type R spacer is about 50 nucleotides in length.

Unless indicated otherwise, type R spacers useful for the present disclosure are not limited to any specific nucleotide sequences, as long as the type R spacers are of sufficient length to carry out their intended function (e.g., aid in the binding of the type R sensors to their ligands). Accordingly, in some aspects, a type R spacer useful for the present disclosure comprises a randomly generated nucleotide sequence. In some aspects, where a plurality of type R spacers are used in separating a plurality of type R sensors, one or more of the plurality of type R spacers have a difference sequence, such that the plurality of type R spacers do not include randomly generated nucleotide sequences that repeat. In some aspects, a type R spacer useful for the present disclosure comprises, consists essentially of, or consists of the sequence tttcctttcccccttccctttttcctttcctttcctttcccccttccctt (SEQ ID NO: 1) or a fragment thereof. In some aspects, the type R spacer comprises the sequence set forth in SEQ ID NO: 1. In some aspects, the type R spacer consists essentially of the sequence set forth in SEQ ID NO: 1. In some aspects, the type R spacer consists of the sequence set forth in SEQ ID NO: 1. In some aspects, a type R spacer useful for the present disclosure comprises, consists essentially of, or consists of the sequence tttcctttcccccttccctt (SEQ ID NO: 2) or a fragment thereof. In some aspects, the type R spacer comprises the sequence set forth in SEQ ID NO: 2. In some aspects, the type R spacer consists essentially of the sequence set forth in SEQ ID NO: 2. In some aspects, the type R spacer consists of the sequence set forth in SEQ ID NO: 2. In some aspects, a type R spacer useful for the present disclosure comprises, consists essentially of, or consists of the sequence gcggccgctaaa (SEQ ID NO: 3) or a fragment thereof In some aspects, the type R spacer comprises the sequence set forth in SEQ ID NO: 3. In some aspects, the type R spacer consists essentially of the sequence set forth in SEQ ID NO: 3. In some aspects, the type R spacer consists of the sequence set forth in SEQ ID NO: 3. In some aspects, the type R spacer is the same as the type P spacer. In some aspects, the type R spacer is different than the type P spacer.

TABLE 2
Exemplary Type R Spacers

| | |
|---|---|
| SEQ ID NO: 1 | tttcctttcccccttccctttttcctttcctttcc |
| | tttcccccttccctt |
| SEQ ID NO: 2 | tttcctttcccccttccctt |
| SEQ ID NO: 3 | gcggccgctaaa |

### Markers

As is apparent from the present disclosure, synthetic circuits described herein can be programmed to selectively regulate the expression of a particular gene (or a protein encoded thereof) in a target cell. Not to be bound by any one theory, in some aspects, because the payload sequence and/or the regulator sequence comprise a sensor (e.g., type P sensor or type R sensor) that has been programmed to recognize a specific marker, the sensor is “turned on” (i.e., in an active form) only in cells that comprise sufficient level of the marker to be recognized by the sensor. Where a cell does not comprise sufficient level of the marker, the sensor is “turned off” (i.e., in an inactive form) as the sensor does not specifically recognize the marker. The below table summarizes the possible scenarios with regard to marker level and payload expression. The status of regulator expression is also listed in the table. Unless indicated otherwise, a marker useful for the present disclosure does not comprise a regulator as described herein.

TABLE 3

| | All Type P | Any Type R | | |
|---|---|---|---|---|
| | Markers are | Marker is | Regulator | Payload |
| Scenario | Low | High | Expression | Expression |
| 1 | False | False | ON | OFF |
| 2 | False | True | OFF | OFF |
| 3 | True | False | ON | OFF |
| 4 | True | True | OFF | ON |

To help illustrate, in some aspects, a synthetic circuit provided herein comprises a payload sequence, wherein the payload sequence comprises a type P sensor that is capable of specifically recognizing a marker expressed in a non-target cell, and wherein the recognition of the marker by the type P sensor reduces or inhibits the expression of the encoded payload in the non-target cell. For such a synthetic circuit, when introduced into the non-target cell (i.e., expresses sufficient level of the marker to be recognized by the type P sensor), the type P sensor becomes active (i.e., bound to the marker) and thereby, inhibits or reduces the expression of the encoded payload in the non-target cell. However, when introduced into a target cell (i.e., does not express sufficient level of the marker to be recognized by the type P sensor), the type P sensor remains inactive (i.e., not bound to a ligand) and therefore, the payload is expressed in the target cell. As is apparent from the present disclosure, payload can be selectively expressed when both of the following are true: (1) NONE of the second type P sensors are activated, and (2) one or more of the type R sensors is/are activated. When either or both of the following two conditions are not met, payload expression can be inhibited:
(1) ALL of the type P markers are low
(2) at least one of the type R markers is high.

As used herein, “expression of the payload” (or grammatical equivalent thereof) refers to any of the following: (a) amount of the payload expressed in the cell, (b) how quickly the payload is expressed in the cell, (c) duration of the payload expression, or (d) any combination of (a) to (c). Not to be bound by any one theory, in some aspects, a synthetic circuit provided herein allows for the selective expression of a payload in a target cell by modulating the expression of the payload and regulator sequences. For instance, as is apparent from the present disclosure, when introduced into a target cell (i.e., does not express sufficient level of a type P marker to be recognized by the type P sensor and expresses sufficient level of a type R marker to be recognized by at least one type R sensor), the expression of the payload in the target cell is increased as compared to the expression of the regulator in the target cell. In some aspects, the expression of the payload in the target cell is increased by at least about 10%, at least about 20%, at least about 30%, at least about 40%, at least about 50%, at least about 60%, at least about 70%, at least about 80%, at least about 90%, or at least about 100%, as compared to the expression of the regulator in the target cell. In some aspects, the expression of the payload in the target cell is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 20-fold, at least about 25-fold, at least about 30-fold, at least about 35-fold, at least about 40-fold, at least about 45-fold, or at least about 50-fold, as compared to the expression of the regulator. In some aspects, the expression of the regulator in the target cell is decreased in the target cell as compared to the expression of the payload. In some aspects, the expression of the regulator in the target cell is decreased by at least about 5%, at least about 10%, at least about 20%, at least about 30%, at least about 40%, at least about 50%, at least about 60%, at least about 70%, at least about 80%, at least about 90%, or about 100%, as compared to the expression of the payload in the target cell.

Unless indicated otherwise, a marker comprises any molecule that is expressed in a cell and can be specifically recognized by a sensor provided herein (e.g., type P sensor and/or type R sensor). As described herein, in some aspects, a marker is selectively expressed (or expressed to a sufficient level) in certain cells but not in other cells. For instance, in some aspects, a marker is expressed to a sufficient level to be recognized by a sensor (e.g., type P sensor or type R sensor) in a first cell but not in a second cell. For such aspects, when a synthetic circuit described herein is introduced into the first cell, the sensor (e.g., type P sensor and/or type R sensor) specifically recognizes the marker and becomes active. When such a synthetic circuit is introduced into the second cell, the sensor (e.g., type P sensor and/or type R sensor) remains inactive (i.e., not bound to a marker).

Non-limiting examples of markers that can be used with the present disclosure include a microRNA (miRNA), a protein, a metabolite, or combinations thereof. In some aspects, the marker comprises a miRNA. In some aspects, a marker comprises a protein. In some aspects, a marker comprises a metabolite.

As further explained elsewhere in the present disclosure, a synthetic circuit provided herein can comprise a plurality of sensors. For instance, in some aspects, a synthetic circuit comprises a payload sequence, wherein the payload sequence comprises a plurality of sensors (e.g., plurality of first type P sensor and/or plurality of second type P sensor). In some aspects, a synthetic circuit comprises a regulator sequence, wherein the regulator sequence comprises a plurality of sensors. In some aspects, a synthetic circuit comprises a payload sequence and a regulator sequence, wherein the payload sequence comprises a plurality of sensors, and wherein the regulator sequence comprises a plurality of sensors. Where a synthetic circuit comprises a plurality of sensors, in some aspects, each of the plurality of sensors can specifically recognize the same marker. For example, in some aspects, the payload sequence of a synthetic circuit provided herein comprises a plurality of sensors, wherein each of the plurality of sensors recognizes the same miRNA. Where a synthetic circuit comprises a plurality of sensors, in some aspects, one or more of the plurality of sensors specifically recognizes different markers. For example, in some aspects, a synthetic circuit comprises a payload sequence and a regulator sequence, wherein the payload sequence and the regulator sequence each comprise a sensor, wherein the sensor of the payload sequence specifically recognizes a first marker (e.g., miRNA) and wherein the sensor of the regulator sequence specifically recognizes a second marker (e.g., metabolite or a different miRNA).

### Regulators

As described herein, in some aspects, synthetic circuits described herein comprise a payload sequence, wherein the payload sequence comprises a sensor that is capable of specifically recognizing a regulator. Unless indicated otherwise, the specific recognition of the regulator by a type P sensor (e.g., first type P sensor) reduces or inhibits the expression of the payload encoded by the payload sequence. Accordingly, where a payload sequence comprises both a sensor that is capable of specifically recognizing a regulator (e.g., first type P sensor) and a sensor that is capable of specifically recognizing a marker (e.g., second type P sensor), the expression of the encoded payload can be regulated by at least two different manners. Not to be bound by any one theory, in some aspects, such a dual approach to regulation allows for greater selectivity in the expression of the payload.

In some aspects, when a synthetic circuit described herein is introduced into a non-target cell (i.e., does not express sufficient level of a type R marker to be recognized by the type R sensor) such that the expression of the regulator is increased, the regulators are capable of being specifically recognized by a type P sensor (e.g., first type P sensor), resulting in the activation of the type P sensor. As further described herein, activation of the type P sensor reduces or inhibits the expression of the payload encoded by the payload sequence. Accordingly, in some aspects, when a synthetic circuit provided herein is contacted with a population of cells comprising both target cells and non-target cells, the expression of the payload in the non-target cell (i.e., has increased expression of the regulator) is decreased by at least about 5%, at least about 10%, at least about 20%, at least about 30%, at least about 40%, at least about 50%, at least about 60%, at least about 70%, at least about 80%, at least about 90%, or about 100%, as compared to the target cell (i.e., has reduced expression of the regulator). In some aspects, when a synthetic circuit provided herein is contacted with a population of cells comprising both target cells and non-target cells, the expression of the payload in the target cell (i.e., has reduced expression of the regulator) is increased by at least about 10%, at least about 20%, at least about 30%, at least about 40%, at least about 50%, at least about 60%, at least about 70%, at least about 80%, at least about 90%, or at least about 100%, as compared to the corresponding expression in the non-target cell. In some aspects, as compared to the non-target cell, the expression of the payload in the target cell is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 20-fold, at least about 25-fold, at least about 30-fold, at least about 35-fold, at least about 40-fold, at least about 45-fold, or at least about 50-fold.

Regulators that can be used with the synthetic circuits described herein comprise any suitable regulators in the art. Non-limiting examples of such regulators include a RNA-binding protein, siRNA, shRNA, pri-miRNAs, ribozymes, or combinations thereof. In some aspects, the regulator is a siRNA. In some aspects, the regulator is a shRNA. In some aspects, the regulator is a pri-miRNA. In some aspects, the regulator is a ribozyme. In some aspects, the regulator is a RNA-binding protein. In some aspects, the RNA-binding protein comprises a ribonuclease. In some aspects, the ribonuclease comprises a Cas protein. In some aspects, the Cas protein comprises a Cas6 protein.

### Exemplary Synthetic Circuits

In some aspects, the regulator sequence in the synthetic circuit comprises an RNA binding domain of PUF-based engineered RNA endonucleases. In some aspects, a synthetic circuit provided herein comprises a PUE-based engineered RNA endonucleases with reprogrammed PUF RNA binding domains (RBDs). Pumilio/fem-3 mRNA binding factor (PUF) proteins are eukaryotic RNA-binding proteins (RBPs) that are involved in post-transcriptional gene regulation. The RNA-binding region of the human Pumilio 1 (PUM1) protein has 8 structural repeats (R1-R8), which recognizes the 8nt target RNA sequence NRE: 5′-UGUAUAUA-3′, which is also referred to herein as a PUF target site (PUF TS). The N-terminal repeat (R1) binds to the 3′-nucleotide residue (N8) of the target sequence, while the C-terminal repeat (R8) binds to the 5′-nucleotide residue (NI).

In some aspects, the PUF RBD useful for the synthetic circuit comprises one or more amino acid substitutions compared to the wild type PUF RBD as set forth in SEQ ID NO: 6. In some aspects, the PUF RBD useful for the synthetic circuit comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the wild type PUM-RBD as set forth in SEQ ID NO: 6, wherein the PUF RBD is capable of binding to the PUF TS.

In some aspects, the first type P sensor comprising a target site that is capable of being specifically bound by a PUF RBD (first type P sensor) comprises 5′-UGUAUAUA-3′ (PUF WT TS), 5′-UGGAUGAA-3′ (PUF TS #1), 5′-UGUACGUC-3′ (PUF TS #2), 5′-UCUACGUC-3′ (PUF TS #3), 5′-UGUACGAC-3′ (PUF TS #4), 5′-UGUCCGUC-3′ (PUF TS #5), 5′-UGUACGUG 3′ (PUF TS #6), 5′-UGGAAGUC-3′ (PUF TS #7), 5′-UGUGCCUC-3′ (PUF TS #8), 5′-UGUAGCU A-3′ (PUF TS #9), or 5′-UGUAGCUA-3′ (TS #10). In some aspects, the TS comprises 5′-UGUAUAUA-3′ (PUF WT TS). In some aspects, the TS comprises 5′-UGGAUGAA-3′ (PUF TS #1). In some aspects, the TS comprises 5′-UGUACGUC-3′ (PUF TS #2). In some aspects, the TS comprises 5′-UCUACGUC-3′ (PUF TS #3). In some aspects, the TS comprises 5′-UGUACGAC-3′ (PUF TS #4). In some aspects, the TS comprises 5′-UGUCCGUC-3′ (PUF TS #5). In some aspects, the TS comprises 5′-UGUACGUG-3′ (PUF TS #6). In some aspects, the TS comprises 5′-UGGAAGUC-3′ (PUF TS #7). In some aspects, the TS comprises 5′-UGUGCCUC-3′ (PUF TS #9). In some aspects, the TS comprises 5′-UGUGCCUC-3′ (PUF TS #8). In some aspects, the TS comprises 5′-UGUAGCUA-3′ (PUF TS #10).

In some aspects, the amino acid substitution in the PUF RBD comprises a substitution corresponding to an amino acid position in R1, R2, R3, R4, R5, R6, R7, or R8 of the wild type PUF RBD as set forth in SEQ ID NO: 6, or any combination thereof. In some aspects, the amino acid substitution in the PUF RBD is in R1. In some aspects, the amino acid substitution in the PUF RBD is in R2. In some aspects, the amino acid substitution in the PUF RBD is in R3. In some aspects, the amino acid substitution in the PUF RBD is in R4. In some aspects, the amino acid substitution in the PUF RBD is in R5. In some aspects, the amino acid substitution in the PUF RBD is in R6. In some aspects, the amino acid substitution in the PUF RBD is in R7. In some aspects, the amino acid substitution in the PUF RBD is in R8. In some aspects, the amino acid substitution in the PUF RBD is capable of binding to the PUF TS with greater affinity than the wild type PUF RBD as set forth in SEQ ID NO: 6. In some aspects, the amino acid substitution in the PUF RBD has less off-target binding than the wild type PUF RBD as set forth in SEQ ID NO: 6.

In some aspects, the regulator sequence in the synthetic circuit comprises a PUF RBD linked to an effector domain, wherein upon binding of PUF RBD to its target site, the effector domain is capable of degrading or reducing the expression of the payload sequence. In some aspects, an effector domain comprises a degradation domain. In some aspects, an effector domain comprises cNOT7. cNOT7 is known as CCR4-NOT transcription complex subunit 7. cNOT7 (uniprot no. Q9UIV1) is a deadenylase that has 3′-5′ poly(A) exoribonuclease activity for synthetic poly(A) RNA substrate. Catalytic component of the CCR4-NOT complex which is one of the major cellular mRNA deadenylases and is linked to various cellular processes including bulk mRNA degradation, miRNA-mediated repression, translational repression during translational initiation and general transcription regulation. During miRNA-mediated repression the complex seems also to act as translational repressor during translational initiation. Additional complex functions may be a consequence of its influence on mRNA expression. cNOT7 associates with members of the BTG family such as TOB1 and BTG2 and is required for their anti-proliferative activity.

In some aspects, cNOT comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence as set forth in SEQ ID NO: 7 (MPAATVDHSQRICEVWACNLDEEMKKIRQVIRKYNYVAMDTEFPGVVARPIGEFRSNADY QYQLLRCNVDLLKIIQLGLTFMNEQGEYPPGTSTWQFNFKFNLTEDMYAQDSIELLTTSGIQ FKKHEEEGIETQYFAELLMTSGVVLCEGVKWLSFHSGYDFGYLIKILTNSNLPEEELDFFEIL RLFFPVIYDVKYLMKSCKNLKGGLQEVAEQLELERIGPQHQAGSDSLLTGMAFFKMREMFF EDHIDDAKYCGHLYGLGSGSSYVQNGTGNAYEEEANKQSV). In some aspects, cNOT7 comprises the amino acid sequence as set forth in SEQ ID NO: 7.

In some aspects, the effector domain comprises TTP (ZFP36). TTP (uniprot no. P26651) is known as mRNA decay activator protein ZFP36. TTP is a zinc-finger RNA-binding protein that destabilizes several cytoplasmic AU-rich element (ARE)-containing mRNA transcripts by promoting their poly(A) tail removal or deadenylation, and hence provide a mechanism for attenuating protein synthesis. TTP can also act as an 3′-untranslated region (UTR) ARE mRNA-binding adapter protein to communicate signaling events to the mRNA decay machinery. TTP recruits deadenylase CNOT7 (and probably the CCR4-NOT complex) via association with CNOT1, and hence promotes ARE-mediated mRNA deadenylation. TTP can also functions also by recruiting components of the cytoplasmic RNA decay machinery to the bound ARE-containing mRNAs, self regulates by destabilizing its own mRNA, binds to 3′-UTR ARE of numerous mRNAs and of its own mRNA, plays a role in anti-inflammatory responses; suppresses tumor necrosis factor (TNF)-alpha production by stimulating ARE-mediated TNF-alpha mRNA decay and several other inflammatory ARE-containing mRNAs in interferon (IFN)- and/or lipopolysaccharide (LPS)-induced macrophages (By similarity), plays a role in the regulation of dendritic cell maturation at the post-transcriptional level, and hence operates as part of a negative feedback loop to limit the inflammatory response, promotes ARE-mediated mRNA decay of hypoxia-inducible factor HIF1A mRNA during the response of endothelial cells to hypoxia, positively regulates early adipogenesis of preadipocytes by promoting ARE-mediated mRNA decay of immediate early genes (IEGs) (By similarity), negatively regulates hematopoietic/erythroid cell differentiation by promoting ARE-mediated mRNA decay of the transcription factor STAT5B mRNA, or plays a role in maintaining skeletal muscle satellite cell quiescence by promoting ARE-mediated mRNA decay of the myogenic determination factor MYOD1 mRNA (By similarity).

In some aspects, TTP comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence as set forth in SEQ ID NO: 8 (MDLTAIYESLLSLSPDVPVPSDHGGTESSPGWGSSGPWSLSPSDSSPSGVTSRLPGRSTSLVE GRSCGWVPPPPGFAPLAPRLGPELSPSPTSPTATSTTPSRYKTELCRTFSESGRCRYGAKCQFA HGLGELRQANRHPKYKTELRHKFYLQGRCPYGSRCHFIHNPSEDLAAPGHPPVLRQSISFSGL PSGRRTSPPPPGLAGPSLSSSSFSPSSSPPPPGDLPLSPSAFSAAPGTPLARRDPTPVCCPSCRRA TPISVWGPLGGLVRTPSVQSLGSDPDEYASSGSSLGGSDSPVFEAGVFAPPQPVAAPRRLPIFN RISVSE). In some aspects, TTP comprises the amino acid sequence as set forth in SEQ ID NO: 8.

In some aspects, the effector domain comprises DDX6 (uniprot no P26196) is known as Probable ATP-dependent RNA helicase DDX6. DDX6 is a helicase involved in decapping and deadenylation. DDX6 is essential for the formation of P-bodies, cytosolic membrane-less ribonucleoprotein granules involved in RNA metabolism through the coordinated storage of mRNAs encoding regulatory functions, plays a role in P-bodies to coordinate the storage of translationally inactive mRNAs in the cytoplasm and prevent their degradation, in the process of mRNA degradation, plays a role in mRNA decapping, and blocks autophagy in nutrient-rich conditions by repressing the expression of ATG-related genes through degradation of their transcripts.

In some aspects, DDX6 comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence as set forth in SEQ ID NO: 9 (MSTARTENPVIMGLSSQNGQLRGPVKPTGGPGGGGTQTQQQMNQLKNTNTINNGTQQQA QSMTTTIKPGDDWKKTLKLPPKDLRIKTSDVTSTKGNEFEDYCLKRELLMGIFEMGWEKPS PIQEESIPIALSGRDILARAKNGTGKSGAYLIPLLERLDLKKDNIQAMVIVPTRELALQVSQICI QVSKHMGGAKVMATTGGTNLRDDIMRLDDTVHVVIATPGRILDLIKKGVAKVDHVQMIVL DEADKLLSQDFVQIMEDIILTLPKNRQILLYSATFPLSVQKFMNSHLQKPYEINLMEELTLKG VTQYYAYVTERQKVHCLNTLFSRLQINQSIIFCNSSQRVELLAKKISQLGYSCFYIHAKMRQ EHRNRVFHDFRNGLCRNLVCTDLFTRGIDIQAVNVVINFDFPKLAETYLHRIGRSGRFGHLG LAINLITYDDRFNLKSIEEQLGTEIKPIPSNIDKSLYVAEYHSEPVEDEKP). In some aspects, DDX6 comprises the amino acid sequence as set forth in SEQ ID NO: 9.

In some aspects, the effector domain comprises an endonuclease domain of MCPIP1 (MCPIP1PIN). MCPIP is known as Endoribonuclease ZC3H12A (uniprot no. Q5D1E8). In some aspects, MCPIP1PIN comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence as set forth in SEQ ID NO: 10 (MGGGTPKAPNLEPPLPEEEKEGSDLRPVVIDGSNVAMSHGNKEVFSCRGILLAVNWFLERG HTDITVFVPSWRKEQPRPDVPITDQHILRELEKKKILVFTPSRRVGGKRVVCYDDRFIVKLAY ESDGIVVSNDTYRDLQGERQEWKRFIEERLLMYSFVNDKFMPPDDPLGRHGPSLDNFLRKK PLTLE). In some aspects, MCPIP1PIN comprises the amino acid sequence as set forth in SEQ ID NO: 10.

### Modalities

As further described herein, synthetic circuits of the present disclosure comprise certain properties (e.g., structural and/or functional) that make them particularly useful for selectively regulating the expression of a gene (or a protein encoded thereof) in a cell of interest (e.g., target cell). For example, in some aspects, a synthetic circuit provided herein comprise a payload sequence and a regulator sequence, which have been programmed such that when both are present in a target cell, the payload (encoded by the payload sequence) is robustly expressed while expression of the regulator (encoded by the regulator sequence) is robustly reduced or inhibited. As described earlier in the present disclosure, in some aspects, a synthetic circuit described herein comprises a sensor (e.g., type P sensor and/or type R sensor), which can be programmed to allow for the selective expression of the payload or regulator in specific cells of interest. In some aspects, in addition to such sensors, a synthetic circuit provided herein comprises a payload sequence and a regulator sequence, wherein the payload sequence and/or the regulator sequence are of a particular modality which is conducive in promoting the selective expression of the payload and/or regulator.

Unless indicated otherwise, a payload sequence comprises one or more of the following RNA modalities: linear RNA, circular RNA, self-replicating RNA, and non-replicating RNA. Unless indicated otherwise, a regulator sequence comprises one or more of the following RNA modalities: linear RNA, circular RNA, and non-replicating RNA. For example, in some aspects, a synthetic circuit provided herein comprises a payload sequence, wherein the payload sequence is a self-replicating RNA. In some aspects, a synthetic circuit provided herein comprises a regulator sequence, wherein the regulator sequence is a non-replicating RNA. Accordingly, in some aspects, a synthetic circuit provided herein comprises a payload sequence and a regulator sequence, wherein the payload sequence is a self-replicating RNA and the regulator sequence is a non-replicating RNA. In some aspects, a synthetic circuit provided herein comprises a payload sequence and a regulator sequence, wherein the payload sequence is a self-replicating RNA and the regulator sequence is a circular RNA (i.e., not self-replicating).

Using self-replicating RNA to express payload sequence while using non-replicating RNA to express the regulator sequence improves performance of the RNA circuit.

Without wishing to be bound by a certain theory, expressing the payload sequence from repRNAs, under marker conditions that enable the payload sequence “ON state”, improves performance of the circuit given repRNAs will replicate and robustly express high levels of payload protein for a long duration. This may be due to the self-replicating nature of self-replicating RNA.

Without wishing to be bound by a certain theory, expressing the regulator sequence from linear RNA instead of repRNAs improves performance of the circuit given:
(1) non-replicating linear RNA can rapidly express sufficient levels of regulator protein to effectively inhibit payload protein expression (from repRNAs), whereas the expression from repRNA is slower (e.g., requires replication) and may allow the payload repRNA to initiate replication, thereby causing “leaky expression” of payload in non-target cells; and
(2) once repRNA starts to replicate, it is more difficult to knock-down (e.g., with miRNAs); therefore, compared to when expressing regulator protein from linear RNA, there may be “leaky expression” of regulator protein in target cells, and this may significantly reduce expression from payload repRNA.

Thus, the combination of repRNA payload sequence with linear RNA regulator sequence allows for very strong expression of payload in target cells, while minimizing expression of transgenes (payload) in non-target cells. Similar to the situation with using linear RNA to regulate a repRNA payload, utilizing a linear RNA regulator strand to regulate a circular RNA payload may also achieve an advantageous outcome, in that circular RNA is more durable than linear RNA.

As is apparent from the present disclosure, a synthetic circuit useful for the present disclosure can comprise various combinations of RNA modalities, so long as the payload can be selectively expressed in the cell of interest (i.e., target cell).

For example, in some aspects, a synthetic circuit provided herein comprises: (a) a first sequence encoding a payload (payload sequence) and (b) a second sequence encoding a regulator (regulator sequence); wherein the payload sequence is a self-replicating RNA and comprises a sensor that is capable of specifically recognizing the regulator (type P sensor); and wherein the regulator sequence is a non-replicating RNA and comprises a sensor that is capable of specifically recognizing a marker (type R sensor). In some aspects, a synthetic circuit provided herein comprises: (a) a first sequence encoding a payload (payload sequence) and (b) a second sequence encoding a regulator (regulator sequence); wherein the payload sequence is a self-replicating RNA and comprises (i) a first sensor that is capable of specifically recognizing the regulator (first type P sensor) and (ii) a second sensor that is capable of specifically recognizing a marker (second type P sensor); and wherein the regulator sequence is a non-replicating RNA and comprises a sensor that is capable of specifically recognizing a marker (type R sensor).

In some aspects, a synthetic circuit provided herein comprises: (a) a first sequence encoding a payload (payload sequence) and (b) a second sequence encoding a regulator (regulator sequence); wherein the payload sequence is a self-replicating RNA and comprises a sensor that is capable of specifically recognizing the regulator (type P sensor); and wherein the regulator sequence is a circular RNA and comprises a sensor that is capable of specifically recognizing a marker (type R sensor). In some aspects, a synthetic circuit provided herein comprises: (a) a first sequence encoding a payload (payload sequence) and (b) a second sequence encoding a regulator (regulator sequence); wherein the payload sequence is a self-replicating RNA and comprises (i) a first sensor that is capable of specifically recognizing the regulator (first type P sensor) and (ii) a second sensor that is capable of specifically recognizing a marker (second type P sensor); and wherein the regulator sequence is a circular RNA and comprises a sensor that is capable of specifically recognizing a marker (type R sensor).

In some aspects, a synthetic circuit provided herein comprises: (a) a first sequence encoding a payload (payload sequence) and (b) a second sequence encoding a regulator (regulator sequence); wherein the payload sequence is a circular RNA and comprises a sensor that is capable of specifically recognizing the regulator (type P sensor); and wherein the regulator sequence is a circular RNA and comprises a sensor that is capable of specifically recognizing a marker (type R sensor). In some aspects, a synthetic circuit provided herein comprises: (a) a first sequence encoding a payload (payload sequence) and (b) a second sequence encoding a regulator (regulator sequence); wherein the payload sequence is a circular RNA and comprises (i) a first sensor that is capable of specifically recognizing the regulator (first type P sensor) and (ii) a second sensor that is capable of specifically recognizing a marker (second type P sensor); and wherein the regulator sequence is a circular RNA and comprises a sensor that is capable of specifically recognizing a marker (type R sensor). In some aspects, a synthetic circuit provided herein comprises: (a) a first sequence encoding a payload (payload sequence) and (b) a second sequence encoding a regulator (regulator sequence); wherein the payload sequence is a circular RNA and comprises a sensor that is capable of specifically recognizing the regulator (type P sensor); and wherein the regulator sequence is a non-replicating RNA and comprises a sensor that is capable of specifically recognizing a marker (type R sensor). In some aspects, a synthetic circuit provided herein comprises: (a) a first sequence encoding a payload (payload sequence) and (b) a second sequence encoding a regulator (regulator sequence); wherein the payload sequence is a non-replicating RNA and comprises (i) a first sensor that is capable of specifically recognizing the regulator (first type P sensor) and (ii) a second sensor that is capable of specifically recognizing a marker (second type P sensor); and wherein the regulator sequence is a non-replicating RNA and comprises a sensor that is capable of specifically recognizing a marker (type R sensor).

### Additional Components

In some aspects, a synthetic circuit described herein comprises one or more additional components that aid in the function of the synthetic circuit. For instance, in some aspects, a synthetic circuit described herein comprises a payload sequence, wherein the payload sequence comprises a type P sensor and one or more additional components described herein. In some aspects, a synthetic circuit described herein comprises a regulator sequence, wherein the regulator sequence comprises a type R sensor and one or more additional components described herein. In some aspects, a synthetic circuit described herein comprise a payload sequence and a regulator sequence, wherein each of the payload sequence and the regulator sequence comprises one or more additional components described herein.

In some aspects, a payload sequence useful for the present disclosure comprises one or more additional components, wherein the one or more additional components enhance the expression of the encoded payload. In some aspects, the regulator sequence does not comprise one or more additional components that enhance the expression of the encoded regulator. Therefore, in some aspects, when such a synthetic circuit is introduced into a target cell, the expression of the payload is increased as compared to the expression of the regulator. In some aspects, compared to the expression of the regulator, the expression of the payload is increased by at least about 10%, at least about 20%, at least about 30%, at least about 40%, at least about 50%, at least about 60%, at least about 70%, at least about 80%, at least about 90%, or at least about 100%. In some aspects, the expression of the payload is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 20-fold, at least about 25-fold, at least about 30-fold, at least about 35-fold, at least about 40-fold, at least about 45-fold, or at least about 50-fold.

In some aspects, a payload sequence useful for the present disclosure comprises one or more additional components that increases the stability of the payload sequence. In some aspects, the regulator sequence does not comprise one or more additional components that increase the stability of the payload sequence. In some aspects, increased stability results in increased expression of the encoded protein. In some aspects, when such a synthetic circuit is introduced into a target cell, the expression of the payload is increased by at least about 10%, at least about 20%, at least about 30%, at least about 40%, at least about 50%, at least about 60%, at least about 70%, at least about 80%, at least about 90%, or at least about 100%. In some aspects, the expression of the payload is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 20-fold, at least about 25-fold, at least about 30-fold, at least about 35-fold, at least about 40-fold, at least about 45-fold, or at least about 50-fold.

In some aspects, the one or more additional components that can be included in a synthetic circuit provided herein comprises an aptamer for a translational initiation factor. For instance, in some aspects, a synthetic circuit provided herein comprises a payload sequence, wherein the payload sequence is a circular RNA and comprises an aptamer for a translational initiation factor. The inclusion of such additional component, particularly where the payload sequence is a circular RNA, can aid in the expression of the encoded payload when the synthetic circuit is introduced into a target cell. See, e.g., Prats et a/., Int J Mol Sci 21(22): 8591 (Nov. 2020). Non-limiting examples of additional components that are useful for the present disclosure include: (1) an internal ribosome entry cite (IRES), (2) an untranslated region (UTR), (3) a sequence encoding a signal peptide, (4) a translation initiation sequence, (5) a polyA sequence, (6) a sequence encoding a RNA binding protein, (7) a sequence encoding a 2A ribosome skip peptide, (8) a 5′-cap, (9) a translation enhancer element, or (10) any combination of (1) to (10). Additional disclosure related to such additional components are provided below.

### Terminal Architecture Modifications: Untranslated Regions (UTRs)

In some aspects, a synthetic circuit described herein comprises a UTR. For example, in some aspects, a synthetic circuit described herein comprises a payload sequence, wherein the payload sequence comprises a UTR. In some aspects, the UTR is a 5′-UTR. In some aspects, the UTR is a 3′-UTR. In some aspects, the UTR comprises both a 5′-UTR and a 3′-UTR.

Untranslated regions (UTRs) of a gene are transcribed but not translated. The 5′-UTR starts at the transcription start site and continues to the start codon but does not include the start codon; whereas, the 3′-UTR starts immediately following the stop codon and continues until the transcriptional termination signal. There is growing body of evidence about the regulatory roles played by the UTRs in terms of stability of the nucleic acid molecule and translation. Accordingly, where a payload sequence described herein comprises a UTR, the stability of the payload sequence is increased, e.g., as compared to a sequence without the UTR. As described herein, in some aspects, increased stability results in increased expression of the encoded protein.

### 5′-UTR and Translation Initiation

Natural 5′-UTRs bear features which play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′-UTR also have been known to form secondary structures which are involved in elongation factor binding.

5′-UTR secondary structures involved in elongation factor binding can interact with other RNA binding molecules in the 5′-UTR or 3′-UTR to regulate gene expression. For example, the elongation factor EIF4A2 binding to a secondarily structured element in the 5′-UTR is necessary for microRNA mediated repression (Meijer H A et al., Science, 2013, 340, 82-85, herein incorporated by reference in its entirety). The different secondary structures in the 5′-UTR can be incorporated into the flanking region to either stabilize or selectively destalized mRNAs in specific tissues or cells.

By engineering the features typically found in abundantly expressed genes of specific target organs, one can enhance the stability and protein production of a nucleic acid sequence (e.g., payload sequence of a synthetic circuit provided herein). For example, introduction of 5′-UTR of liver-expressed mRNA, such as albumin, serum amyloid A, Apolipoprotein A/B/E, transferrin, alpha fetoprotein, erythropoietin, or Factor VIII, could be used to enhance expression of a nucleic acid molecule, such as a mRNA, in hepatic cell lines or liver. Likewise, use of 5′-UTR from other tissue-specific mRNA to improve expression in that tissue is possible—for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-l, i-NOS), for leukocytes (CD45, CD18), for adipose tissue (CD36, GLUT4, ACRP30, adiponectin) and for lung epithelial cells (SP-A/B/C/D).

Other non-UTR sequences can also be incorporated into the UTRs (e.g., 5′-UTR and/or 3′-UTR). For example, introns or portions of introns sequences can be incorporated into the flanking regions of a nucleic acid sequence (e.g., payload sequence of a synthetic circuit provided herein).

In some aspects, one or more nucleotides within a UTR (e.g., 5′-UTR and/or 3′-UTR) can be mutated, replaced and/or removed. For example, one or more nucleotides upstream of the start codon can be replaced with another nucleotide. The nucleotide or nucleotides to be replaced can be 1, 2,3,4, 5,6,7,8,9, 10, 11, 12, 13, 14, 15, 16, 17, 18, 19, 20, 21, 22, 23, 24, 25, 30, 35, 40, 45, 50, 55, 60 or more than 60 nucleotides upstream of the start codon. As another example, one or more nucleotides upstream of the start codon can be removed from the UTR.

### 3′ UTR and the AU Rich Elements

3′-UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′-UTR of nucleic acid molecules can lead to HuR binding and thus, stabilization of the message in vivo.

In some aspects, introduction, removal, or modification of 3′-UTR AU rich elements (AREs) can be used to modulate the stability of a nucleic acid sequence. When engineering specific nucleic acid sequences (e.g., payload sequence and/or regulator sequence described herein), one or more copies of an ARE can be introduced to make the nucleic acid sequence less stable and thereby curtail translation and decrease production of the resultant protein. Likewise, AREs can be identified and removed or mutated to increase the intracellular stability and thus increase translation and production of the resultant protein.

### Translation Enhancer Elements (TEEs)

In some aspects, a synthetic circuit provided herein comprises a translational enhancer element (TEE). As used herein, the term “translational enhancer element” refers to cis-acting sequences that increase the expression of a protein encoded by a nucleotide sequence. Non-limiting examples of TEEs that can be used with the present disclosure are known in the art, see, e.g., US20130177581A, which is incorporated herein by reference in its entirety. In some aspects, a synthetic circuit provided herein comprises a payload sequence and a regulator sequence, wherein the payload sequence comprises a TEE. When such a synthetic circuit is introduced into a target cell, the expression of the payload is increased, e.g., as compared to a corresponding synthetic circuit where the payload sequence does not comprise the TEE.

In some aspects, the TEE is positioned between the transcription promoter and the start codon of a sequence (e.g., payload sequence). In some aspects, a TEE useful for the present disclosure has at least about 50%, at least about 55%, at least about 60%, at least about 65%, at least about 70%, at least about 75%, at least about 80%, at least about 85%, at least about 90%, at least about 95%, or at least about 99% sequence identity with any of the TEEs provided in U.S. Publication Number US 20140147454, US20090226470, US20070048776, US20130177581, US20110124100, WO1999024595, WO2012009644, WO2009075886, WO2007025008, U.S. Pat. Nos. 6,310,197, 6,849,405, 7,456,273, 7,183,395, each of which is herein incorporated by reference in its entirety.

In some aspects, a synthetic circuit provided herein comprises multiple TEEs. For example, in some aspects, a synthetic circuit provided herein comprises a payload sequence, wherein the payload sequence comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55, or more than 60 TEE sequences. In some aspects, the TEE sequences in the 5′UTR of the RNA (e.g., modified RNA) are the same or different TEE sequences. In some aspects, the TEE sequences are in a pattern such as ABABAB or AABBAABBAABB or ABCABCABC or variants thereof repeated once, twice, or more than three times. In these patterns, each letter, A, B, or C represent a different TEE sequence at the nucleotide level.

### RNA Binding Proteins (RBPs)

In some aspects, a synthetic circuit provided herein comprises a sequence encoding a RNA binding protein. RNA binding proteins (RBPs) can regulate numerous aspects of co- and post-transcription gene expression such as, but not limited to, RNA splicing, localization, translation, turnover, polyadenylation, capping, modification, export and localization. RNA-binding domains (RBDs), such as, but not limited to, RNA recognition motif (RR) and hnRNP K-homology (KH) domains, typically regulate the sequence association between RBPs and their RNA targets (Ray et al., Nature 2013. 499:172-177; herein incorporated by reference in its entirety). In some aspects, the canonical RBDs bind short RNA sequences. In some aspects, the canonical RBDs recognize RNA structure.

Non limiting examples of RNA binding proteins and related nucleic acid and protein sequences are described in US 2014/0147454, which is herein incorporated by reference in its entirety.

### 5′ Capping

In some aspects, a synthetic circuit described herein comprises a 5-cap structure. For example, in some aspects, a synthetic circuit described herein comprises a payload sequence, wherein the payload sequence comprises a 5′-cap structure. The 5′ cap structure of a mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Modifications to the RNA of the present disclosure can generate a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with a-thio-guanosine nucleotides according to the manufacturer's instructions to create a phosphorothioate linkage in the 5′-ppp-5′ cap. Additional modified guanosine nucleotides can be used such as a-methyl-phosphonate and seleno-phosphate nucleotides.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs can be chemically (i.e. non-enzymatically) or enzymatically synthesized and/linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′ mppp-G; which can equivalently be designated 3′ O-Me-m7G(5′)ppp(5′)G). The 3′-O atom of the other, unmodified, guanine becomes linked to the 5′-terminal nucleotide of the capped nucleic acid molecule (e.g., an mRNA or mmRNA). The N7- and 3′-O-methylated guanine provides the terminal moiety of the capped nucleic acid molecule (e.g., mRNA or mmRNA).

Another exemplary cap is mCAP, which is similar to ARCA but has a 2′-β-methyl group on guanosine (i.e., N7,2′-O-dimethyl-guanosine-5′-triphosphate-5′-guanosine, m7Gm-ppp-G).

In some aspects, the cap is a dinucleotide cap analog. In some aspects, the dinucleotide cap analog is modified at different phosphate positions with a boranophosphate group or a phosphoroselenoate group such as the dinucleotide cap analogs described in U.S. Pat. No. 8,519,110, the contents of which are herein incorporated by reference in its entirety.

In some aspects, the cap is a cap analog is a N7-(4-chlorophenoxyethyl) substituted dicucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See e.g., the various cap analogs and the methods of synthesizing cap analogs described in Kore et al. Bioorganic & Medicinal Chemistry 2013 21:4570-4574; the contents of which are herein incorporated by reference in its entirety). In some aspects, a cap analog of the present disclosure is a 4-chloro/bromophenoxyethyl analog.

While cap analogs allow for the concomitant capping of a nucleic acid molecule in an in vitro transcription reaction, up to about 20% of transcripts remain uncapped. This, as well as the structural differences of a cap analog from an endogenous 5′-cap structures of nucleic acids produced by the endogenous, cellular transcription machinery, can lead to reduced translational competency and reduced cellular stability.

In some aspects, providing an RNA with a 5′-cap or 5′-cap analog is achieved by in vitro transcription of a DNA template in the presence of said 5′-cap or 5′-cap analog, wherein said 5′-cap is co-transcriptionally incorporated into the generated RNA strand,

In some aspects, RNA can be generated, for example, by in vitro transcription, and the 5′-cap can be attached to the RNA post-transcriptionally using capping enzymes, for example, capping enzymes of vaccinia virus. In some aspects, the nucleotide sequence encoding IL-12 is capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′ cap structures of the present disclosure are those which, among other things, have enhanced binding of cap binding proteins, increased half-life, reduced susceptibility to 5′ endonucleases and/or reduced 5′ decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. This cap results in a higher translational-competency and cellular stability and a reduced activation of cellular pro-inflammatory cytokines, as compared, e.g., to other 5′ cap analog structures known in the art. Cap structures include 7mG(5′)ppp(5′)N,pN2p, 7mG(5′)ppp(5′)NlmpNp, 7mG(5′)-ppp(5′)NlmpN2 mp and m(7)Gpppm(3)(6,6,2′)Apm(2′)Apm(2′)Cpm(2)(3,2′)Up.

In some aspects, 5′ terminal caps include endogenous caps or cap analogs. In some aspects, a 5′ terminal cap comprises a guanine analog. Useful guanine analogs include inosine, NI-methyl-guanosine, 2′ fluoro-guanosine, 7-deaza-guanosine, 8-oxo-guanosine, 2-amino-guanosine, LNA-guanosine, and 2-azido-guanosine.

In some aspects, the 5′ cap comprises a 5′ to 5′ triphosphate linkage. In some aspects, the 5′ cap comprises a 5′ to 5′ triphosphate linkage including thiophosphate modification. In some aspects, the 5′ cap comprises a 2′-O or 3′-O-ribose-methylated nucleotide. In some aspects, the 5′ cap comprises a modified guanosine nucleotide or modified adenosine nucleotide. In some aspects, the 5′ cap comprises 7-methylguanylate. Exemplary cap structures include m7G(5′)ppp(5′)G, m7,2′O-mG(5′)ppSp(5′)G, m7G(5′)ppp(5′)2′O-mG, and m7,3′O-mG(5′)ppp(5′)2′O-mA.

In some aspects, a synthetic circuit described herein comprises a modified 5′ cap. For instance, in some aspects, the payload sequence of a synthetic circuit comprises a modified 5-cap. A modification on the 5′ cap can increase the stability of mRNA, increase the half-life of the mRNA, and could increase the mRNA translational efficiency. In some aspects, the modified 5′ cap comprises one or more of the following modifications: modification at the 2′ and/or 3′ position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

The 5′ cap structure that can be modified includes, but is not limited to, the caps described in U.S. Application No. 2014/0147454 and WO2018/160540 which is incorporated herein by reference in its entirety.

### IRES Sequences

In some aspects, a synthetic circuit provided herein comprises an internal ribosome entry site (IRES). For example, in some aspects, a synthetic circuit provided herein comprises a payload sequence, wherein the payload sequence comprises an IRES. First identified as a feature Picorna virus RNA, IRES plays an important role in initiating protein synthesis in absence of the 5′ cap structure. An IRES can act as the sole ribosome binding site, or can serve as one of multiple ribosome binding sites of an mRNA. Nucleic acids or mRNA containing more than one functional ribosome binding site can encode several peptides or polypeptides that are translated independently by the ribosomes (“multicistronic nucleic acid molecules”). When nucleic acids or mRNA are provided with an IRES, further optionally provided is a second translatable region. Examples of IRES sequences that can be used according to the disclosure include without limitation, those from picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Poly-A Tails

In some aspects, a synthetic circuit provided herein comprises a poly-A tail. In some aspects, a synthetic circuit provided herein comprises a payload sequence, wherein the payload sequence comprises a poly-A tail.

In some aspects, the length of the poly-A tail is greater than about 30 nucleotides in length. In some aspects, the poly-A tail is greater than about 35 nucleotides in length. In some aspects, the length is at least about 40 nucleotides. In some aspects, the length is at least about 45 nucleotides. In some aspects, the length is at least about 55 nucleotides. In some aspects, the length is at least about 60 nucleotides. In some aspects, the length is at least 70 nucleotides. In some aspects, the length is at least about 80 nucleotides. In some aspects, the length is at least about 90 nucleotides. In some aspects, the length is at least about 100 nucleotides. In some aspects, the length is at least about 120 nucleotides. In some aspects, the length is at least about 140 nucleotides. In some aspects, the length is at least about 160 nucleotides. In some aspects, the length is at least about 180 nucleotides. In some aspects, the length is at least about 200 nucleotides. In some aspects, the length is at least about 250 nucleotides. In some aspects, the length is at least about 300 nucleotides. In some aspects, the length is at least about 350 nucleotides. In some aspects, the length is at least about 400 nucleotides. In some aspects, the length is at least about 450 nucleotides. In some aspects, the length is at least about 500 nucleotides. In some aspects, the length is at least about 600 nucleotides. In some aspects, the length is at least about 700 nucleotides. In some aspects, the length is at least about 800 nucleotides. In some aspects, the length is at least about 900 nucleotides. In some aspects, the length is at least about 1000 nucleotides. In some aspects, the length is at least about 1100 nucleotides. In some aspects, the length is at least about 1200 nucleotides. In some aspects, the length is at least about 1300 nucleotides. In some aspects, the length is at least about 1400 nucleotides. In some aspects, the length is at least about 1500 nucleotides. In some aspects, the length is at least about 1600 nucleotides. In some aspects, the length is at least about 1700 nucleotides. In some aspects, the length is at least about 1800 nucleotides. In some aspects, the length is at least about 1900 nucleotides. In some aspects, the length is at least about 2000 nucleotides. In some aspects, the length is at least about 2500 nucleotides. In some aspects, the length is at least about 3000 nucleotides.

In some aspects, the poly-A tail comprises a polyA-G quartet. The G-quartet is a cyclic hydrogen bonded array of four guanine nucleotides that can be formed by G-rich sequences in both DNA and RNA. In some aspects, the G-quartet is incorporated at the end of the poly-A tail. The resultant nucleic acid or mRNA can be assayed for stability, protein production and other parameters including half-life at various time points. It has been discovered that the polyA-G quartet results in protein production equivalent to at least 75% of that seen using a poly-A tail of 120 nucleotides alone.

### Modified Nucleosides

In some aspects, a synthetic circuit provided herein comprises one or more modified nucleosides. In some aspects, a synthetic circuit provided herein comprises a payload sequence, wherein the payload sequence comprises one or more modified nucleosides. In some aspects, the one or more modified nucleosides comprises 6-aza-cytidine, 2-thio-cytidine, a-thio-cytidine, pseudo-iso-cytidine, 5-aminoallyl-uridine, 5-iodo-uridine, NI-methyl-pseudouridine, 5,6-dihydrouridine, a-thio-uridine, 4-thio-uridine, 6-aza-uridine, 5-hydroxy-uridine, deoxy-thymidine, pseudo-uridine, inosine, a-thio-guanosine, 8-oxo-guanosine, 06-methyl-guanosine, 7-deaza-guanosine, N1-methyl adenosine, 2-amino-6-chloro-purine, N6-methyl-2-amino-purine, 6-chloro-purine, N6-methyl-adenosine, a-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, and combinations thereof.

In some aspects, a synthetic circuit provided herein comprises one or more uridines which have been replaced by a modified nucleoside. In some aspects, the modified nucleoside replacing uridine is pseudouridine (ψ), N1-methyl-pseudouridine (m1ψ) or 5-methyl-uridine (m5U).

### Nanoparticle and Delivery System

In some aspects, the present disclosure relates to the delivery of a synthetic circuit (e.g., described herein) to cells. In some aspects, the delivery can occur in vivo (e.g., by administering a synthetic circuit described herein to a subject) or ex vivo (e.g., by culturing a synthetic circuit described herein with the cells in vitro). In some aspects, delivery of a synthetic circuit described herein can be performed using any suitable delivery system known in the art. In certain aspects, the delivery system is a vector. Accordingly, in some aspects, the present disclosure provides a vector comprising any of the synthetic circuits described herein. Suitable vectors that can be used are known in the art. See, e.g., Sung et al., Biomater Res 23(8) (2019) which is incorporated herein by reference in its entirety.

In some aspects, a synthetic circuit is delivered using a nanoparticle (e.g., lipid nanoparticle or lipid like nanoparticle). Accordingly, in some aspects, the present disclosure relates to a synthetic circuit (e.g., described herein) encapsulated within a nanoparticle, a composition comprising such a nanoparticle, and the use of such a nanoparticle to treat a disease or disorder in a subject in need thereof. More specifically, in some aspects, provided herein is a nanoparticle comprising (i) any of the synthetic circuits described herein and (ii) one or more types of nanoparticle components.

### Nanoparticle (NP)

A “nanoparticle” (NP), as used herein, refers to a particle, such as a vesicle, having characteristic dimensions measured in nanometers (nm). Nanoparticles can be used in methods by which pharmaceutical therapies are delivered to targeted locations. Non-limiting examples of NPs include lipid nanoparticles (LNPs), lipid-like nanoparticles (LLNs), polymeric nanoparticles (PNPs), and inorganic nanoparticles.

### Lipid Nanoparticle (LNP)

A “lipid nanoparticle” (LNP), as used herein, refers to a nanoparticle composed of lipids. Lipid nanoparticles can be used in methods by which pharmaceutical therapies are delivered to targeted locations. Non-limiting examples of LNPs include cationic lipid nanoparticles, ionizable lipid nanoparticles, liposomes, bolaamphihiles, solid lipid nanoparticles (SLN), nanostructured lipid carriers (NLC), and monolayer membrane structures (e.g., archaeosomes and micelles).

As used herein a “cationic lipid nanoparticle” refers to a nanoparticle comprising a cationic lipid. As used herein an “ionizable lipid nanoparticle” refers to a nanoparticle comprising an ionizable lipid. In aspects of the disclosure, LNPs comprise one or more of the following lipids: a “non-cationic helper lipid,” a “phospholipid,” a “sterol other structural lipid,” and a “PEG/pegylated lipid.”

Exemplary LNPs comprise one or more of the following components:
(i) an ionizable/cationic lipid;
(ii) phospholipid or a non-cationic helper lipid;
(iii) a sterol or other structural lipid;
(iv) a PEG/PEGylated lipid and
(v) a targeted delivery molecule (lipid composition/targeting ligand).

### Lipid Like Nanoparticle (LLN)

A “lipid like nanoparticle” (LLN), as used herein, refers to a nanoparticle comprising a lipid, and a lipid-like material or a lipidoid, as described herein. In aspects of the disclosure, LLNs comprise one or more of the following lipids: a “non-cationic helper lipid,” a “phospholipid,” a “sterol other structural lipid”, and a “PEG/PEGylated lipid.” Lipid like nanoparticles can be used in methods by which pharmaceutical therapies are delivered to targeted locations.

Exemplary LLNs comprise one or more of the following components:
(i) an ionizable/cationic lipid-like material or lipidoid;
(ii) phospholipid or a non-cationic helper lipid;
(iii) a sterol or other structural lipid;
(iv) a PEG/PEGylated lipid; and
(v) a targeted delivery molecule (lipid composition/targeting ligand).

### Ionizable Lipid

Non-limiting examples of ionizable lipids include: Non-limiting examples of ionizable lipids include: ((4-hydroxybutyl)azanediyl)bis(hexane-6,1-diyl)bis(2-hexyldecanoate) (ALC-0315), heptadecan-9-yl 8-((2-hydroxyethyl)(6-oxo-6-(undecyloxy)hexyl)amino) octanoate (SM-102), heptadecan-9-yl 8-((2-hydroxyethyl)(8-(nonyloxy)-8-oxooctyl)amino)octanoate (Lipid 5), di((Z)-non-2-en-1-yl) 9-((4-(dimethylamino)butanoyl)oxy)heptadecanedioate (L319), 3-(didodecylamino)-N1,N1,4 tridodecyl-1-piperazineethanamine (KL10), Nl-[2 (didodecylamino)ethyl]-N1,N4,N4-tridodecyl 1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-l9-yl-4-(dimethylamino)butanoate (DLin-MC3-DMA), 2,2-dilinoleyl-4-(2 dimethylaminoethyl)-[1,3]-dioxolane (DLin-KC2-DMA), 1,2-dioleyloxy-N,N-dimethylaminopropane (DODMA), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3j3)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3 β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), or any combination thereof.

### Cationic Lipid

Non-limiting examples of a cationic lipid include: 1,2-dioleoyl-3-trimethylammonium-propane (DOTAP), lipofectamine, N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTEVI), 2,3-dioleyloxy-N-[2(sperminecarboxamido)ethyl]-N,N-dimethyl-l-propanaminium trifluoroacetate (DOSPA), N,N-distearyl-N,N-dimethylammonium bromide (DDAB), N-(1,2-dimyristyloxyprop-3-yl)-N,N-dimethyl-N-hydroxy ethyl ammonium bromide (DMRIE), N-(1,2-dioleoyloxyprop-3-yl)-N,N-dimethyl-N-hydroxy ethyl ammonium bromide (DORIE), N,N-dioleyl-N,N-dimethylammonium chloride (DODAC), 1,2-dilauroyl-sn-glycero-3-ethylphosphocholine (DLePC), 1,2-distearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP), 1,2-distearoyl-sn-glycero-3-ethylphosphocholine (DSePC), 1,2-dipalmitoyl-sn-glycero-3-ethylphosphocholine (DPePC), 1,2-dimyristoyl-sn-glycero-3-ethylphosphocholine (DMePC), 1,2-dioleoyl-sn-glycero-3-ethylphosphocholine (DOePC), 1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), or any combination thereof.

In some aspects of the disclosure, LNPs primarily comprise cationic lipids along with other lipid ingredients. These typically include other lipid molecules belonging but not limited to the phophatidylcholine (PC) class (e.g., 1,s-Distearoyl-sn-glycero-3-phophocholine (DSPC), and 1,2-Dioleoyl-sn-glycero-3-phophoethanolamine (DOPE), sterols (e.g., cholesterol) and Polyethylene glycol (PEG)-lipid conjugates (e.g., 1,2-distearoyl-sn-glycero-3-phosphoethanolamine-N-[folate(polyethylene glycol)-2000 (DSPE-PEG2000), and C14-PEG2000.

### DOTAP

In aspects of the disclosure, the catonic lipid is DOTAP. DOTAP can be used for the highly efficient transfection of DNA including yeast artificial chromosomes (YACs) into eukaryotic cells for transient or stable gene expression, and is also suitable for the efficient transfer of other negatively charged molecules, such as RNA, oligonucleotides, nucleotides, ribonucleoprotein (RNP) complexes, and proteins into research samples of mammalian cells.

### Lipofectamine

In aspects of the disclosure, the catonic lipid is lipofectamine. Lipofectamine, as used herein, is a common transfection reagent, produced and sold by Invitrogen, used in molecular and cellular biology. It is used to increase the transfection efficiency of RNA (including mRNA and siRNA) or plasmid DNA into in vitro cell cultures by lipofection. Lipofectamine contains lipid subunits that can form liposomes or lipid nanoparticles in an aqueous environment, which entrap the transfection payload, e.g., modRNA. The RNA-containing liposomes (positively charged on their surface) can fuse with the negatively charged plasma membrane of living cells, due to the neutral co-lipid mediating fusion of the liposome with the cell membrane, allowing nucleic acid cargo molecules to cross into the cytoplasm for replication or expression.

### Lipid-Like Material or Lipidoid

Unless indicated otherwise, “lipid-like material” and “lipidoid” can be used interchangeably. Non-limiting examples of lipid-like materials and/or lipidoids include: 1,1′-((2-(4-(2-((2-(bis(2-hydroxydodecyl) amino)ethyl) (2-hydroxydodecyl)amino)ethyl) piperazin-1-yl)ethyl)azanediyl) bis(dodecan-2-ol) (C12-200), 3,6-bis(4-(bis(2-hydroxydodecyl)amino)butyl)piperazine2,5-dione (cKK-E12), tetrakis(8-methylnonyl) 3,3′,3″,3′″-(((methylazanediyl) bis(propane-3,1 diyl))bis (azanetriyl))tetrapropionate (306Oi₁₀), G0-C14, 5A2-SC8, 3,6-bis(4-(bis((9Z,12Z)-2-hydroxyoctadeca9,12-dien-1-yl)amino)butyl)piperazine-2,5-dione (OF-02), (((3,6-dioxopiperazine-2,5-diyl)bis(butane-4,1-diyl))bis(azanetriyl))tetrakis(ethane2,1-diyl) (9Z,9′Z,9″Z,9′Z,12Z,12′Z,12″Z,12′″Z)-tetrakis (octadeca-9,12-dienoate) (OF-Deg-Lin), (((3,6-dioxopiperazine-2,5-diyl)bis(butane-4,1-diyl)) bis(azanetriyl))tetrakis (butane-4,1-diyl) (9Z,9′Z,9″Z,9′″Z,12Z,12′Z,12″Z,12′″Z)-tetrakis (octadeca-9,12-dienoate) (OF-C4-Deg-Lin), NI,N3,N5-tris(3-(didodecylamino)propyl)benzenel,3,5-tricarboxamide (TT3), Hexa(octan-3-yl) 9,9′,9″,9′″,9″″,9′″″-((((benzene-1,3,5-tricarbonyl)ris(azanediyl)) tris (propane-3,1-diyl))tris(azanetriyl))hexanonanoate (FTT5), PL-1 [disclosed in Nature Communications, 12-7264 (2021), which is is hereby incorporated by reference], 98N12-5 [disclosed in Molecular Therapy vol. 17 no. 5 May 2009, which is hereby incorporated by reference], ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18 (A2)) and A12-Iso5-2DC18 (A12), or any combination thereof.

TT3, as used herein, is capable of forming nanoparticles for delivery of various biologic active agents into the cells. In addition, the present disclosure also demonstrates that an unloaded TT3-LLN can induce immunogenic cell death (ICD) in cancer cells in vivo and in vitro. Immunogenic cell death, as described herein, refers to a form of cell death that can induce an effective immune response through activation of dendritic cells (DCs) and consequent activation of specific T cell response. In some aspects of the disclosure, the cells that undergo immunogenic cell death are tumor cells. Immunogenic tumor cell death can trigger an effective anti-tumor immune response.

In some aspects, the lipidoid is TT3.

### Phospholipid, or Other Non-Cationic Helper Lipid

Unless indicated otherwise, “phospholipid” and “other non-cationic helper lipid” can be used interchangeably and non-limiting examples include: 1,2-dilinoleoyl-sn-glycero-3 phosphocholine (DLPC), 1,2-dimyristoyl-sn-glycerol-phosphocholine (DMPC), 1,2-dioleoyl-sn glycerol-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and any combinations thereof.

In some embodiments, the phospholipid is selected from the group consisting of 1-myristoyl-2-palmitoyl-sn-glycero-3-phosphocholine (14:0-16:0 PC, MPPC), 1-myristoyl-2 stearoyl-sn-glycero-3-phosphocholine (14:0-18:0 PC, MSPC), 1-palmitoyl 2-acetyl-sn-glycero-3-phosphocholine (16:0-02:0 PC), 1-palmitoyl-2-myristoyl-sn-glycero-3-phosphocholine (16:0-14:0 PC, PMPC), 1-palmitoyl-2-stearoyl-sn-glycero-3-phosphocholine (16:0-18:0 PC, PSPC), 1-palmitoyl-2-oleoyl-sn-glycero-3-phosphocholine (16:0-18:1 PC, POPC), 1-palmitoyl-2-linoleoyl-sn-glycero-3-phosphocholine (16:0-18:2 PC, PLPC), 1-palmitoyl-2-arachidonoyl-sn-glycero-3-phosphocholine (16:0-20:4 PC), 1-palmitoyl-2-docosahexaenoyl-sn-glycero-3-phosphocholine (14:0-22:6 PC), 1-stearoyl-2-myristoyl-sn-glycero-3-phosphocholine (18:0-14:0 PC, SMPC), 1-stearoyl-2-palmitoyl-sn-glycero-3-phosphocholine (18:0-16:0 PC, SPPC), 1-stearoyl-2-oleoyl-sn-glycero-3-phosphocholine (18:0-18:1 PC, SOPC), 1-stearoyl-2-linoleoyl-sn-glycero-3-phosphocholine (18:0-18:2 PC), 1-stearoyl-2-arachidonoyl-sn-glycero-3-phosphocholine (18:0-20:4 PC), 1-stearoyl-2-docosahexaenoyl-sn-glycero-3-phosphocholine (18:0-22:6 PC), 1-oleoyl-2-myristoyl-sn-glycero-3-phosphocholine (18: 1-14:0 PC, OMPC), 1-oleoyl-2-palmitoyl-sn-glycero-3-phosphocholine (18: 1-16:0 PC, OPPC), 1-oleoyl-2-stearoyl-sn-glycero-3-phosphocholine (18: 1-18:0 PC, OSPC), 1-palmitoyl-2-oleoyl-sn-glycero-3-phosphoethanolamine (16:0-18:1 PE, POPE), 1-palmitoyl-2-linoleoyl-sn-glycero-3-phosphoethanolamine (16:0-18:2 PE), 1-palmitoyl-2-arachidonoyl-sn-glycero-3-phosphoethanolamine (16:0-20:4 PE), 1-palmitoyl-2-docosahexaenoyl-sn-glycero-3-phosphoethanolamine (16:0-22:6 PE), 1-stearoyl-2-oleoyl-sn-glycero-3-phosphoethanolamine (18:0-18:1 PE), 1-stearoyl-2-linoleoyl-sn-glycero-3-phosphoethanolamine (18:0-18:2 PE), 1-stearoyl-2-arachidonoyl-sn-glycero-3-phosphoethanolamine (18:0-20:4 PE), 1-stearoyl-2-docosahexaenoyl-sn-glycero-3-phosphoethanolamine (18:0-22:6 PE), 1-oleoyl-2-cholesterylhemisuccinoyl-sn-glycero-3-phosphocholine (OChemsPC), and any combination thereof.

In some aspects, the phospholipid is DSPC. In some aspects, the phospholipid is DOPE.

### Sterol or Other Structural Lipid

As used herein, a “sterol or other structural lipid,” refers to cholestrol or cholesterol analogs that could be used to fill lipid membrane packing defects and provide structural integrity.

Non-limiting examples of sterols include: a cholesterol, fecosterol, sitosterol, ergosterol, campesterol, stigmasterol, brassicasterol, tomatidine, tomatine, ursolic acid, alpha-tocopherol, and combinations thereof. In some aspects, the sterol is cholesterol.

### PEG Pegylated Lipid

As used herein, a “PEG lipid” and a “pegylated lipid” are used interchangeably.

Non-limiting examples of PEG lipids include: 2-[(polyethylene glycol)-2000]-N,N-ditetradecylacetamide (ALC-0159), 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some aspects, the lipid moiety of the PEG-lipids includes those having lengths of from about C14 to about C22.

In some aspects, the PEG moiety has a size of about 1000, 2000, 5000, 10,000, 15,000 or 20,000 daltons. In certain aspects, C14-PEG2000 comprises 1,2-dimyristoyl-rac-glycero-3-methoxypolyethylene glycol-2000 (DMG-PEG2000), 1,2-dimyristoyl-sn-glycero-3-phosphoethanolamine-N-[methoxy(polyethylene glycol)-2000](DMPE-PEG2000), or both.

In some aspects, the PEG-lipids can be embedded in the LNP prior to the encapsulation of the polynucleotide. In some aspects, the PEG lipids (or other lipid ingredients disclosed herein) can be added to the LNP after the encapsulation of the polynucleotide. For example, in some aspects, a synthetic circuit is encapsulated in the LNP, and then the PEG lipid (other lipid ingredients disclosed herein) is attached to the LNP using, e.g., micelles.

In some aspects, the nanoparticle does not comprise any pegylated lipid. In some aspects, the lipid nanoparticle does not comprise any pegylated lipid.

### Targeted Delivery Molecule (Lipid Composition Targeting Ligand)

In aspects of the disclosure, nanoparticles, (e.g., LNPs and LLNs), as described herein, comprise a targeted delivery molecule (lipid composition/targeting ligand). As used herein, a “targeted delivery molecule (lipid composition/targeting ligand),” and a “targeted delivery molecule” are used interchangeably and in some aspects, the targeted delivery molecule could be an additional lipid or lipid like component, as described herein. In some aspects, the targeted delivery molecule could change the overall charge of the nanoparticles. In some aspects, the targeted delivery molecule could be a non-covalently or covalently bound ligand to the nanoparticle. In some aspects, the targeted delivery ligand could be a small molecule or a large molecule.

Non-limiting examples of targeted delivery molecules are disclosed in Pharmaceuticals (Basel). July 20;15(7):897. (2022), Nat Rev Drug Discov 20, 101-124 (2021), and Advanced Drug Delivery Reviews, Volume 188, (2022), which are hereby incorporated by reference. Non-limiting examples of targeted delivery molecules include: 1,2-dioleoyl-3-trimethylammonium-propane (DOTAP), N,N-distearyl-N,N-dimethylammonium bromide (DDAB), 1,2-dioleoyl-sn-glycero-3-phosphate (sodium salt) (18:1 PA), 1,2-dimyristoyl-sn-glycero-3-phosphate (sodium salt) (14:0 PA), bis(monooleoylglycero)phosphate (S,R Isomer) (ammonium salt) (18BMP), 2-((2,3-bis(oleoyloxy)propyl)dimethylammonio)ethyl ethyl phosphate (DOCPe), folic acid, N-acetylgalactosamine (GalNAc), anti-CD3 antibodies.

### Molar Ratios

In some aspects, a nanoparticle described herein comprises a lipid (e.g., an ionizable lipid, a cationic lipid, a non-cationic helper lipid, a phospholipid, a sterol or other structural lipid, or PEG lipid) and/or lipidoid, as described herein at a molar ratio of about 10% to about 50% in the lipid and/or lipid like composition. In some aspects, a nanoparticle provided herein comprises a lipid and/or lipidoid, as described herein, at a molar ratio of about 10%, about 20%, about 30%, about 40%, or about 50% in the lipid composition.

In some aspects, a nanoparticle provided herein comprises a lipid and/or lipidoid, as described herein, at a molar ratio of about 10% in the lipid and/or lipid like composition. In some aspects, a nanoparticle provided herein comprises a lipid and/or lipidoid, as described herein, at a molar ratio of about 20% in the lipid and/or lipid like composition. In some aspects, a nanoparticle provided herein comprises a lipid and/or lipidoid, as described herein, at a molar ratio of about 30% in the lipid and/or lipid like composition. In some aspects, a nanoparticle provided herein comprises a lipid and/or lipidoid, as described herein, at a molar ratio of about 40% in the lipid and/or lipid like composition. In some aspects, a nanoparticle provided herein comprises a lipid and/or lipidoid, as described herein, at a molar ratio of about 40% in the lipid and/or lipid like composition.

In some aspects, a nanoparticle described herein comprises a pegylated lipid at a molar ratio of about 0% to about 10% in the lipid and/or lipid like composition. In some aspects, a lipid nanoparticle comprises a pegylated lipid at a molar ratio of about 0%, about 0.25%, about 0.5%, about 0.75%, about 1%, about 2%, about 3%, about 4%, about 5%, about 6%, about 7%, about 8%, about 9%, or about 10% in the lipid and/or lipid like composition.

In some aspects, a nanoparticle provided herein comprises a pegylated lipid at a molar ratio of about 0.25% in the lipid and/or lipid like composition. In some aspects, a nanoparticle provided herein comprises a pegylated lipid at a molar ratio of about 0.5% in the lipid and/or lipid like composition. In some aspects, a nanoparticle provided herein comprises a pegylated lipid at a molar ratio of about 0.75% in lipid and/or lipid like composition. In some aspects, a nanoparticle provided herein comprises a pegylated lipid at a molar ratio of about 1.0% in the lipid and/or lipid like composition. In some aspects, a nanoparticle provided herein comprises a pegylated lipid at a molar ratio of about 2.0% in the lipid and/or lipid like composition. In some aspects, a nanoparticle provided herein comprises a pegylated lipid at a molar ratio of about 3.0% in the lipid and/or lipid like composition. In some aspects, a nanoparticle provided herein comprises a pegylated lipid at a molar ratio of about 4.0% in the lipid and/or lipid like composition. In some aspects, a nanoparticle provided herein comprises a pegylated lipid at a molar ratio of about 5.0% in the lipid and/or lipid like composition. In some aspects, a nanoparticle provided herein comprises a pegylated lipid at a molar ratio of about 6.0% in the lipid and/or lipid like composition. In some aspects, a nanoparticle provided herein comprises a pegylated lipid at a molar ratio of about 7.0% in the lipid and/or lipid like composition. In some aspects, a nanoparticle provided herein comprises a pegylated lipid at a molar ratio of about 8.0% in the lipid and/or lipid like composition. In some aspects, a nanoparticle provided herein comprises a pegylated lipid at a molar ratio of about 9.0% in the lipid and/or lipid like composition. In some aspects, a nanoparticle provided herein comprises a pegylated lipid at a molar ratio of about 10.0% in the lipid and/or lipid like composition.

For example, in some aspects, the pegylated lipid comprises C14-PEG2000. In some aspects, the C14-PEG2000 is present in the lipid nanoparticle at a molar ratio of about 0.25% in the lipid and/or lipid like composition. In some aspects, the C14-PEG2000 is present at a molar ratio of about 0.5% in the lipid and/or lipid like composition. In some aspects, the C14-PEG2000 is present at a molar ratio of about 0.75% in the lipid and/or lipid like composition. In some aspects, the C14-PEG2000 is present at a molar ratio of about 1% in the lipid and/or lipid like composition. In some aspects, the C14-PEG2000 is present at a molar ratio of about 2% in the lipid and/or lipid like composition. In some aspects, the C14-PEG2000 is present at a molar ratio of about 3% in the lipid and/or lipid like composition. In some aspects, the C14-PEG2000 is present at a molar ratio of about 4% in the lipid and/or lipid like composition. In some aspects, the C14-PEG2000 is present at a molar ratio of about 5% in the lipid and/or lipid like composition. In some aspects, the C14-PEG2000 is present at a molar ratio of about 6% in the lipid and/or lipid like composition. In some aspects, the C14-PEG2000 is present at a molar ratio of about 7% in the lipid and/or lipid like composition. In some aspects, the C14-PEG2000 is present at a molar ratio of about 8% in the lipid and/or lipid like composition. In some aspects, the C14-PEG2000 is present at a molar ratio of about 9% in the lipid and/or lipid like composition. In some aspects, the C14-PEG2000 is present at a molar ratio of about 10% in the lipid and/or lipid like composition.

### Particle Size

Particle size of nanoparticles can affect drug release rate, bio-distribution, mucoadhesion, cellular uptake of water and buffer exchange to the interior of the nanoparticles, and protein diffusion. In some aspects of the disclosure, the diameter of the NPs ranges from about 30 to about 500 nm. In some aspects of the disclosure, the diameter of the NPs ranges from about 30 to about 500 nm, about 50 to about 400 nm, about 70 to about 300 nm, about 100 to about 200 nm, about 100 to about 175 nm, or about 100 to about 160 nm. In some aspects of the disclosure, the diameter of the NPs ranges from 100-160 nm. In some aspects of the disclosure, the diameter of the NPs can be about 30 nm, about 40 nm, about 50 nm, about 60 nm, about 70 nm, about 80 nm, about 90 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm., about 130 nm, about 140 nm, about 150 nm, or about 160 nm. In certain aspects, the lipid nanoparticle has a diameter of about 140 nm.

### Zeta Potential

As used herein, “zeta potential” refers to the measure of the effective electric charge on the nanoparticle surface. The magnitude of the zeta potential provides information about particle stability. In some aspects of the disclosure, the zeta potential of the nanoparticles ranges from about −20 to about 20 mv. In some aspects of the disclosure, the zeta potential of the NPs can be about −6.0 mv, about −5.9 mv, about −5.8 mv, about −5.7 mv, about −5.6 mv, about −5.5 mv, about −5.4 mv, about −5.3 mv, about −5.2 mv, about −5.1 mv, about −5.0 mv, about −4.9 mv, about −4.8 mv, about −4.7 mv, about −4.6 mv, about −4.5 mv, about −4.4 mv, about −4.3 mv, about −4.2 mv, about −4.1 mv, about −4.0 mv, about −3.9 mv, about −3.8 mv, about −3.7 mv, about −3.6 mv, about −3.5 mv, about −3.4 mv, about −3.3 my, about −3.2 mv, about −3.1 my, about −3.0 mv, about −2.9 mv, about −2.8 mv, about −2.7 my, about −2.6 mv, about −2.5 my, about −2.4 mv, about −2.3 mv, about −2.2 my, about −2.1 mv, about −2.0 mv, about −1.9 mv, about −1.8 mv, about −1.7 mv, about −1.6 mv, about −1.5 mv, about −1.4 mv, about −1.3 my, about −1.2 mv, about −1.1 my, about −1.0 mv, about −0.9 my, about −0.8 mv, about −0.7 my, about −0.6 mv, about −0.5 mv, about −0.4 mv, about −0.3 mv, about −0.2 mv, about −0.1 mv, about 0.0 mv, 0.1 about mv, about 0.2 mv, about 0.3 mv, about 0.4 mv, about 0.5 mv, about 0.6 mv, about 0.7 mv, about 0.8 mv, about 0.9 mv, about 1.0 mv, about 1.1 mv, about 1.2 mv, about 1.3 mv, about 1.4 mv, about 1.5 mv, about 1.6 mv, about 1.7 mv, about 1.8 mv, about 1.9 mv, about 2.0 mv, about 2.1 mv, about 2.2 mv, about 2.3 mv, about 2.4 mv, about 2.5 mv, about 2.6 mv, about 2.7 mv, about 2.8 mv, about 2.9 mv, about 3.0 mv, about 3.1 mv, about 3.2 mv, about 3.3 mv, about 3.4 mv, about 3.5 mv, about 3.6 mv, about 3.7 mv, about 3.8 mv, about 3.9 mv, about 4.0 mv, about 4.1 mv, about 4.2 mv, about 4.3 mv, about 4.4 mv, about 4.5 mv, about 4.6 mv, about 4.7 mv, about 4.8 mv, about 4.9 mv, about 5.0 mv, about 5.1 mv, about 5.2 mv, about 5.3 mv, about 5.4 mv, about 5.5 mv, about 5.6 mv, about 5.7 mv, about 5.8 my, about 5.9 mv, or about 6.0 mv.

### Mass Ratio

In some aspects, the mass ratio between the lipid of the LNPs or the LLN and the synthetic circuit ranges from about 1:2 to about 15:1. In some aspects, the mass ratio between the lipid and the synthetic circuit can be about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, about 10:1, about 10.5:1, about 11:1, about 11.5:1, about 12:1, about 12.5:1, about 13:1, about 13.5:1, about 14:1, about 14.5:1, or about 15:1. In some aspects of the disclosure, the mass ratio between the lipid and the synthetic circuit is about 10:1.

### Pharmaceutical Compositions

In some aspects, the disclosure relates to a pharmaceutical composition comprising a synthetic circuit, vector, and/or nanoparticle described herein. In some aspects, the pharmaceutical composition further comprises a pharmaceutically acceptable carrier (excipient). “Acceptable”, as used herein, means that the carrier must be compatible with the active ingredient of the composition and not deleterious to the subject to be treated. In some aspects, the carrier is capable of stabilizing the active ingredient. Pharmaceutically acceptable excipients (carriers) include buffers, which are well known in the art. See, e.g., Remington: The Science and Practice of Pharmacy 20th Ed. (2000) Lippincott Williams and Wilkoins, Ed. K. E. Hoover.

The pharmaceutical compositions to be used for in vivo administration must be sterile. This is readily accomplished by, for example, filtration through sterile filtration membranes. The nanoparticles can be placed into a container having a sterile access port, for example, an intravenous solution bag or vial having a stopper pierceable by a hypodermic injection needle.

In some aspects, the pharmaceutical composition can be formulated for intratumoral, intrathecal, intramuscular, intravenous, subcutaneous, inhalation, intradermal, intralymphatic, intraocular, intraperitoneal, intrapleural, intraspinal, intravascular, nasal, percutaneous, sublingual, submucosal, transdermal, or transmucosal administration. In some aspects of the disclosure, the pharmaceutical composition can be formulated for intratumoral injection. Intratumoral injection, as used herein, refers to direct injections into the tumor. A high concentration of composition can be achieved in situ, while using small amounts of drugs. Local delivery of immunotherapies allows multiple combination therapies, while preventing significant system exposure and off-target toxicities.

In some aspects, the pharmaceutical composition can be formulated for intramuscular injection, intravenous injection, or subcutaneous injection.

In some aspects, the pharmaceutical composition comprises pharmaceutically acceptable carriers, buffer agents, excipients, salts, or stabilizers in the form of lyophilized formulations or aqueous solutions. See, e.g., Remington: The Science and Practice of Pharmacy 20^{th}Ed. (2000) Lippincott Williams and Wilkins, Ed. K. E. Hoover. Acceptable carriers and excipients, or stabilizers are nontoxic to recipients at the dosages and concentrations used, and comprises buffers such as phosphate, citrate, and other organic acds; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

In some aspects, the pharmaceutical composition described herein comprises nanoparticles which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545, which are hereby incorporated by reference in their entirety. Liposomes with enhanced circulation time are disclosed in U.S. Pat. No. 5,013,556, which is hereby incorporated by reference in its entirety. In some aspects, liposomes can be generated by the reverse phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

In some aspects, the pharmaceutical composition is formulated in sustained-release format. Suitable examples of sustained-release preparations include semipermeable matrices of solid hydrophobic polymers containing the nanoparticles which matrices are in the form of shaped articles, e.g., films or microcapsules. Examples of sustained-release matrices include, but are not limited to, polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPROM DEPOTT™ (injectable microspheres composed of lactic acid-glycolic acid copolymer and leuprolide acetate), sucrose acetate isobutyrate, and poly-D-(−)-3-hydroxybutyric acid.

In some aspects, suitable surface-active agents include, but are not limited to, non-ionic agents, such as polyoxyethylenesorbitans (e.g., TWEEN™ 20, 40, 60, 80 or 85) and other sorbitans (e.g., SPAN™ 20, 30, 60, 80, or 85). In some aspects, compositions with a surface-active agent comprise between 0.05 and 5% surface-active agent. In some aspects the composition comprises 0.1 and 2.5%. It will be appreciated that other ingredients can be added, for example mannitol or other pharmaceutically acceptable vehicles, if necessary.

In some aspects, the pharmaceutical composition is in unit dosage forms such as tablets, pills, capsules, powders, granules, solutions or suspensions, or suppositories, for oral, parenteral, or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogenous mixture of a compound of the present disclosure, or a non-toxic pharmaceutically acceptable salt thereof. When referring to these preformulation compositions as homogeneous, it is meant that the active ingredient is dispersed evenly throughout the composition so that the composition can be readily subdivided into equally effective unit dosage forms such as tablets, pills, and capsules. This solid preformulation composition is then subdivided into unit dosage forms of the type described above containing from about 0.1 to about 500 mg of the active ingredient of the present disclosure. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials include a number of polymeric acids and mixtures of polymeric acids with such materials as shellac, cetyl alcohol, and cellulose acetate.

Suitable emulsions can be prepared using commercially available fat emulsions, such as INTRALIPID™, LIPOSYN™, INFONUTROL™, LIPOFUNDIN™, and LIPIPHYSAN™. The active ingredient can be either dissolved in a pre-mixed emulsion composition or alternatively it can be dissolved in an oil (e.g., soybean oil, safflower oil, cottonseed oil, sesame oil, corn oil, or almond oil) and an emulsion formed upon mixing with a phospholipid (e.g., egg phospholipids, soybean phospholipids, or soybean lecithin) and water. It will be appreciated that other ingredients can be added, for example glycerol or glucose, to adjust tonicity of the emulsion. Suitable emulsions will typically contain up to about 20% oil, for example, between about 5 and about 20%. The fat emulsion can comprise fat droplets having a suitable size and can have a pH in the range of about 5.5 to about 8.0.

Pharmaceutical compositions for inhalation or insufflation include solutions and suspensions in pharmaceutically acceptable, aqueous or organic solvents, or mixtures thereof, and powders. The liquid or solid compositions can contain suitable pharmaceutically acceptable excipients as set out above. In some aspects, the composition is administered by the oral or nasal respiratory route for local or systemic effect.

Compositions in pharmaceutically acceptable solvents can be nebulized by use of gases. Nebulized solutions can be breathed directly from the nebulizing device or the nebulizing device can be attached to a face mask, tent or intermittent positive pressure breathing machine. Solution, suspension, or powder compositions can be administered from devices which deliver the formulation in an appropriate manner.

### Therapeutic Applications

In some aspects of the disclosure, the synthetic circuits, vectors, nanoparticles, and/or pharmaceutical compositions described herein (also collectively referred to herein as “compositions”) are used to treat a disease or disorder. As is apparent from the present disclosure, any of the compositions provided herein can be used to treat a wide range of diseases or disorders. Any suitable disease or disorder whether in a therapeutic agent can be encoded by the payload sequence of a synthetic circuit provided herein. Accordingly, some aspects of the present disclosure relates to a method of treating a disease or disorder in a subject in need thereof, comprising administering to the subject any of the compositions provided herein (e.g., synthetic circuit).

In some aspects, any of the compositions described herein is administered to a subject in need thereof via a suitable route, such as intratumoral administration, intravenous administration (e.g, as a bolus or by continuous infusion over a period of time), by intramuscular, intraperitoneal, intracerebospinal, subcutaneous, intra-articular, intrasynovial, intrathecal, oral, inhalation, or topical routes. Commercially available nebulizers for liquid formulations, including jet nebulizers and ultrasonic nebulizers are useful for administration. Liquid formulations can be nebulized and lyophilized powder can be nebulized after reconstitution. In some aspects, the pharmaceutical composition described herein is aerolized using a fluorocarbon formulation and a metered dose inhaler, or inhaled as a lyophilized and milled powder. In some aspects, the pharmaceutical composition described herein is formulated for intratumoral injection. In some aspects, the pharmaceutical composition described herein is administered to a subject via a local route, for example, injected to a local site such as a tumor site or an infectious site. In some aspects, the subject is a human.

As will be apparent from the present disclosure, in some aspects, the compositions described herein are administered to a subject in an effective amount to confer a therapeutic effect, either alone or in combination with one or more other active agents. In some aspects, the compositions are administered to a subject suffering from a cancer, and the therapeutic effect comprises reduced tumor burden, reduction of cancer cells, increased immune activity, or combinations thereof. Whether the administered composition (e.g., a nanoparticle, such as a LNP or LLN) achieved the therapeutic effect can be determined using any suitable methods known in the art (e.g., measuring tumor volume and/or T cell activity). Effective amounts vary, as recognized by those skilled in the art, depending on the particular condition being treated, the severity of the condition, the individual patient parameters including age, physical condition, size, gender and weight, the duration of the treatment, the nature of the concurrent therapy (if any), the specific route of administration and like factors within the knowledge of expertise of the health practitioner.

Empirical considerations, such as the half-life, generally will contribute to the determination of the dosage. Frequency of administration can be determined and adjusted over the course of therapy, and is generally, but not necessarily, based on treatment and/or suppression and/or amelioration and/or delay of a target disease/disorder. Alternatively, sustained continuous release formulations of a composition described herein (e.g., a nanoparticle, such as a LNP or LLN) can be appropriate. Various formulations and devices for achieving sustained release are known in the art.

In some aspects of the disclosure, the treatment is a single injection of the composition disclosed herein. In some aspects, the single injection is administered intratumorally to the subject in need thereof.

In some aspects of the disclosure, dosages for a composition described herein can be determined empirically in individuals who have been given one or more administration(s) of the composition (e.g., nanoparticle described herein). In some aspects, the individuals are given incremental dosages of the composition described herein. To assess efficacy of the composition herein, an indicator of disease/disorder can be followed. For repeated administrations over several days or longer, depending on the condition, in some aspects, the treatment is sustained until a desired suppression of symptoms occurs or until sufficient therapeutic levels are achieved to alleviate a target disease or disorder, or symptom thereof

In some aspects of the disclosure, the method comprises administering to a subject in need thereof one or multiple doses of a composition described herein.

As described herein, a synthetic circuit of the present disclosure is particularly useful in selectively expressing a payload in a target cell of interest. Accordingly, some aspects of the present disclosure relates to a method of inducing the selective expression of a payload in a cell, comprising contacting a population of cells with any of the compositions provided herein (e.g., synthetic circuit). In some aspects, the payload is expressed in the cell when the cell meets the following condition: (i) comprises a sufficient level of a type R marker such that the type R marker is specifically recognized by the type R sensor and inducing the activation of the type R sensor, thereby, reducing or inhibiting the expression of the regulator; (ii) does not comprise a sufficient level of a type P marker such that the type P marker is not recognized by the type P sensor, allowing the type P sensor to remain in an inactive form; or (iii) both (i) and (ii). When a cell satisfies such conditions, the expression of the payload is increased in the cell as compared to a reference cell (e.g., corresponding cell that does not meet any of the conditions described above). In some aspects, after the contacting, the expression of the payload in the cell is increased by at least about 10%, at least about 20%, at least about 30%, at least about 40%, at least about 50%, at least about 60%, at least about 70%, at least about 80%, at least about 90%, or at least about 100%, as compared to the reference cell. In some aspects, after the contacting, the expression of the payload is increased in the cell by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 20-fold, at least about 25-fold, at least about 30-fold, at least about 35-fold, at least about 40-fold, at least about 45-fold, or at least about 50-fold, as compared to the reference cell.

In some aspects, the present disclosure provides a method of generating immune cells expressing a chimeric antigen receptor (CAR), T cell receptor (TCR) or TCR mimic in vivo in a subject in need thereof comprising administering the synthetic circuit, the vector, the nanoparticle, or the pharmaceutical composition, wherein the synthetic circuit expresses the CAR, TCR, or TCR mimic as a payload.

In some aspects, the present disclosure provides a method of treating cancer in a subject in need thereof by in situ generated immune cells expressing a chimeric antigen receptor (CAR), T cell receptor (TCR) or TCR mimic, comprising administering the synthetic circuit, the vector, the nanoparticle, or the pharmaceutical composition, wherein the synthetic circuit expresses the CAR, TCR, or TCR mimic as a payload.

In some aspects, the CAR expressed by the synthetic circuit for the methods of the present disclosure targets CD19, TRAC, TCRβ, BCMA, CLL-1, CS1, CD38, CD19, TSHR, CD123, CD22, CD30, CD70, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-l lRa, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTl, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3E, CD4, CD5, CD7, the extracellular portion of the APRIL protein, or any combinations thereof.

In some aspects, the TCR expressed by the synthetic circuit for the methods of the present disclosure targets AFP, CD19, TRAC, TCRR, BCMA, CLL-1, CSi, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 lRa, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLECl2A, BST2, EMR2, LY75, GPC3, FCRL5, IGLL1, CD2, CD3F, CD4, CD5, CD7, the extracellular portion of the APRIL protein, or any combinations thereof.

In some aspects, the composition described herein is co-administered with at least one additional suitable therapeutic agent. In some aspects, the composition described herein and the at least one additional therapeutic agent are administered to the subject in a sequential manner, i.e, each therapeutic agent is administered at a different time. In some aspects, the composition described herein and the at least one additional therapeutic agent are administered to the subject in a substantially simultaneous manner.

In some aspects, a therapeutic application of a synthetic circuit described herein comprises producing the encoded payload in a target cell. Accordingly, in some aspects, the present disclosure relates to a method of selectively producing a payload in a target cell. In some aspects, the method comprises contacting a target cell with any of the compositions described herein (e.g., synthetic circuit, vectors, and/or nanoparticles) under conditions suitable for producing the encoded IL-12 protein. In some aspects, the method further comprises purifying the produced payload. In some aspects, the contacting occurs in vivo (e.g., by administering the synthetic circuit, vector, and/or nanoparticle to a subject). In some aspects, the contacting occurs ex vivo (e.g., by culturing cells with the synthetic circuit, vector, and/or nanoparticles in vitro). Cells (e.g., host cells) comprising the synthetic circuit, vector, and/or nanoparticle are encompassed herein. Non-limiting examples of cells that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### Kits for Use in Therapy

The present disclosure also provides kits for use in therapy. In some aspects, the kit includes one or more containers comprising a composition described herein.

In some aspects, the kit comprises instructions for use in accordance with any of the methods described herein. For example, the included instructions can comprise a description of administration of the pharmaceutical composition described herein to treat, delay the onset, or alleviate a target disease. In some aspects, the instructions comprise a description of administering the composition described herein to a subject at risk of a target disease.

In some aspects, the instructions comprise dosage information, dosing schedule, and route of administration. In some aspects, the containers are unit doses, bulk packages (e.g., multi-dose packages) or sub-unit doses. In some aspects, the instructions are written instructions on a label or package insert (e.g., a paper sheet included in the kit). In some aspects, the instructions are machine-readable instructions (e.g., instructions carried on a magnetic or optical storage disk).

In some aspects, the kits described herein are in suitable packaging. In some aspects, suitable packing comprises vials, bottles, jars, flexible packaging (e.g., seal Mylar or plastic bags), or combinations thereof. In some aspects, the packaging comprises packages for use in combination with a specific device such as an inhaler, nasal administration device (e.g., an atomizer), or an infusion device such as a minipump. In some aspects, the kit comprises a sterile access port (for example the container can be an intravenous solution bag or a vial having a stopper pierceable by a hypodermic injection needle). In some aspects, the container can also have a sterile access port (for example the container can be an intravenous solution bag or a vial having a stopper pierceable by a hypodermic injection needle). In some aspects, at least one active agent is a composition as described herein.

In some aspects, the kits further comprise additional components such as buffers and interpretive information. In some aspects, the kit comprises a container and a label or package insert(s) on or associated with the container. In some aspects, the disclosure provides articles of manufacture comprising the contents of the kits described herein.

### General Techniques

The practice of the present disclosure will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Giffiths, and D. G. Newell, eds., 1993-8) J. Wiley and Sons; Method of Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987): PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane, Cold Spring Harbor Laboratory Press, 1999); The Antibodies (M. Zanette and J. D. Capra, eds., Harwood Academic Publishers, 1995). Without further elaboration, it is believed that one skilled in the art can, based on the above description, utilize the present disclosure to its fullest extent. All publications cited herein (including those listed above and elsewhere in the present disclosure) are incorporated by reference in their entirety.

### EXAMPLES

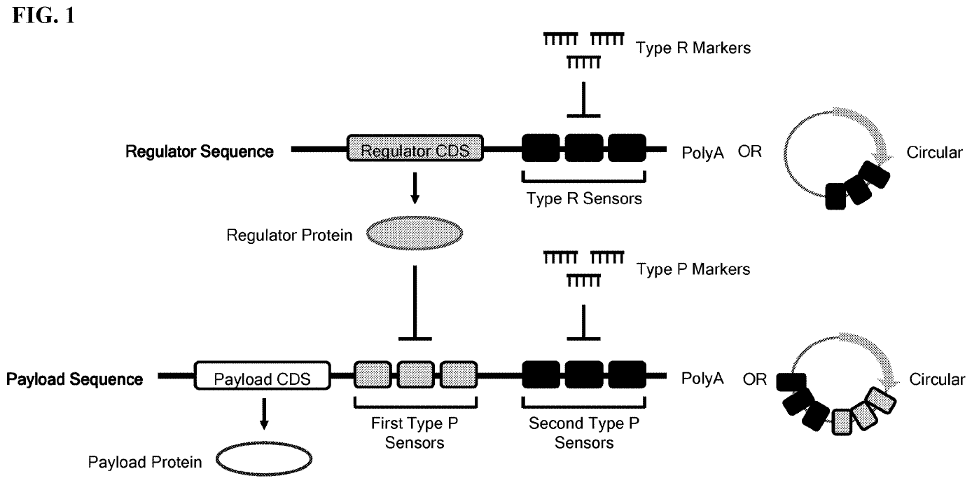

The following experiments (Examples 1-7) describe optimized RNA based logic circuits for better kinetics. The optimized circuit includes, but is not limited to: a polynucleotide sequence encoding a payload (payload sequence), a polynucleotide sequence encoding a regulator (regulator sequence), a regulator sequence sensor that is capable of specifically recognizing a marker (type R sensor), a payload sequence sensor that is capable of specifically recognizing the regulator (first type P sensor), and a payload sequence sensor that is capable of specifically recognizing a marker (second type P sensor), as exemplified in FIG. 1. As shown in FIG. 1, one or more of the protein(s) encoded by the regulator sequence interact with the payload, either to positively or negatively regulate it.

Further, as shown in FIG. 1, the regulator sequence and the payload sequence may comprise linear (self-replicating or non-replicating) or circular RNA.

Key features of the payload sequence within the optimized circuit include, but are not limited to, multiple miRNA classifiers that detarget expression in multiple organs to facilitate systemic delivery and optimized number and placement of miRNA sensors for a potent OFF switch. Key features of the regulator sequence within the optimized circuit include, but are not limited to, highly specific and sensitive binding with quick target engagement to avoid expression in non-target cells, expression levels tuned to ensure better switch behavior, multiple miRNA classifiers that enable payload expression in target cells, and it is fit for purpose based on the gene therapy application.

### Example 1: Single Sensor Repression

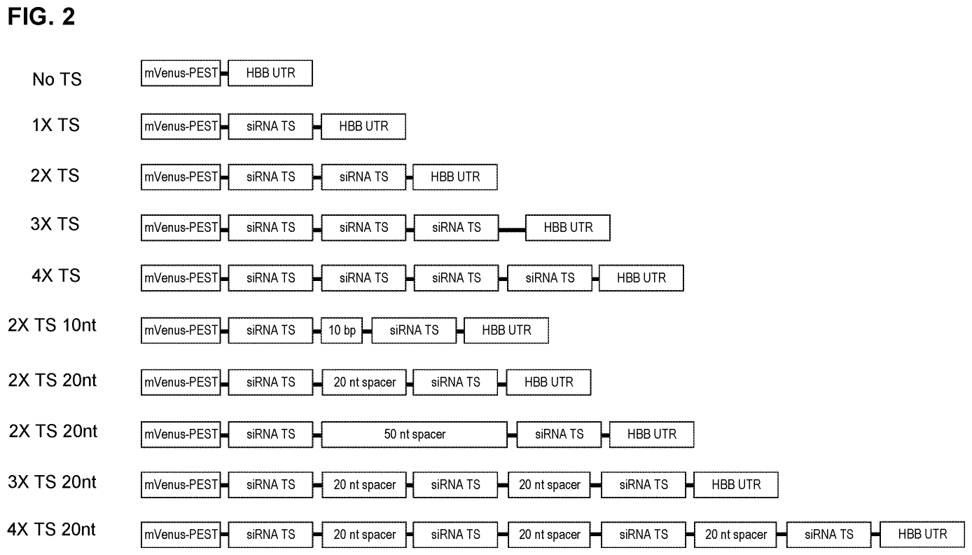

3′UTR target site arrays for single sensors, as shown in FIG. 2 were tested for optimal repression.

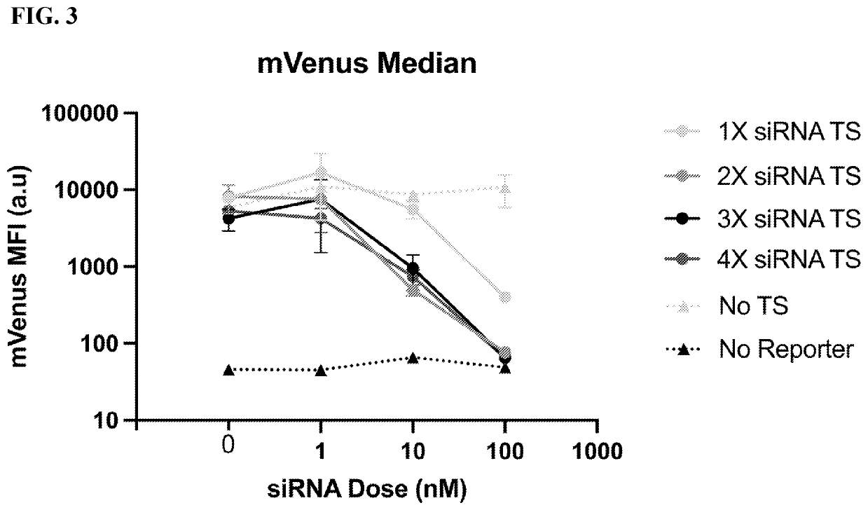

Number of target sites. First, the effect of the number of target sites on repression, as assessed by mVenus median fluorescence arbitrary units (a.u.) for constructs containing either 1X siRNA 2, 2X siRNA 2, 3X siRNA 2, 4X siRNA 2, no target site (TS), or no reporter following administration of siRNA (0, 1, 10, or 100 nM) was determined (FIG. 3). As shown in FIG. 3, modRNA constructs containing 1-4X siRNA 2 target sites immediately following the stop codon of an mVenus-PEST reporter demonstrate preferential knockdown in the HEK293T cells with increased co administered doses of the siRNA 2 siRNA. Incorporation of >2 identical, adjacent target sites does not significantly increase repression regardless of siRNA dose. Data represent the median collected via flow cytometry from three technical replicates (n=3) at 20 hours post electroporation of reporter and siRNA. Data represent the median collected via flow cytometry from three technical replicates (n=3) at 20 hr post electroporation of reporter and siRNA.

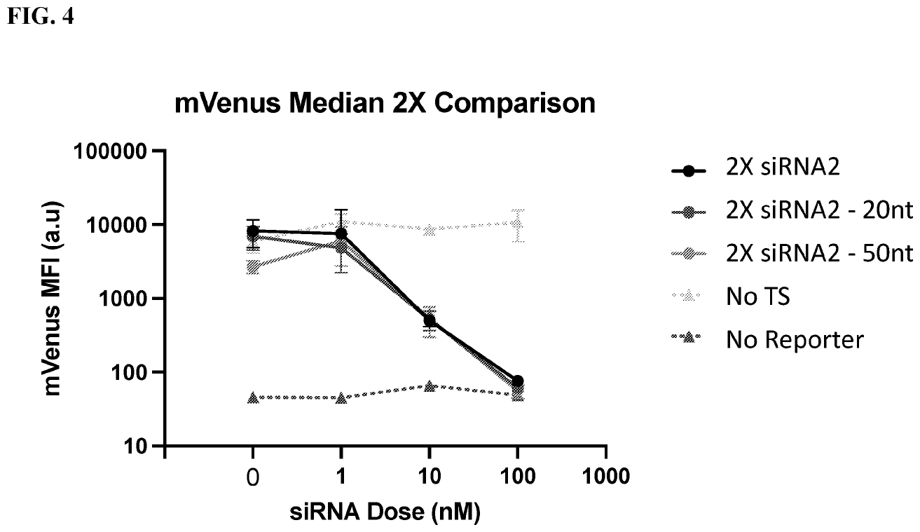

Target site spacing. Next, the effect of target site spacing on repression, as assessed by mVenus median fluorescence (a.u.) for constructs containing either 2X siRNA 2, 2X siRNA 2-20nt, 2X siRNA 2-50nt, no TS, or No Reporter following administration of siRNA (0, 1, 10, or 100 nM) was determined (FIG. 4). Data represent the median collected via flow cytometry from three technical replicates (n=3) at 20 hours post electroporation of reporter and siRNA. As shown in FIG. 4, modRNA constructs containing 2X siRNA 2 target sites with varying spacing between them immediately following the stop codon of an mVenus-PEST reporter demonstrate preferential knockdown in the HEK293T cells with increased co-administered doses of siRNA 2. Incorporation of spacing between the target sites does not significantly increase knockdown for low, medium or high dose of siRNA.

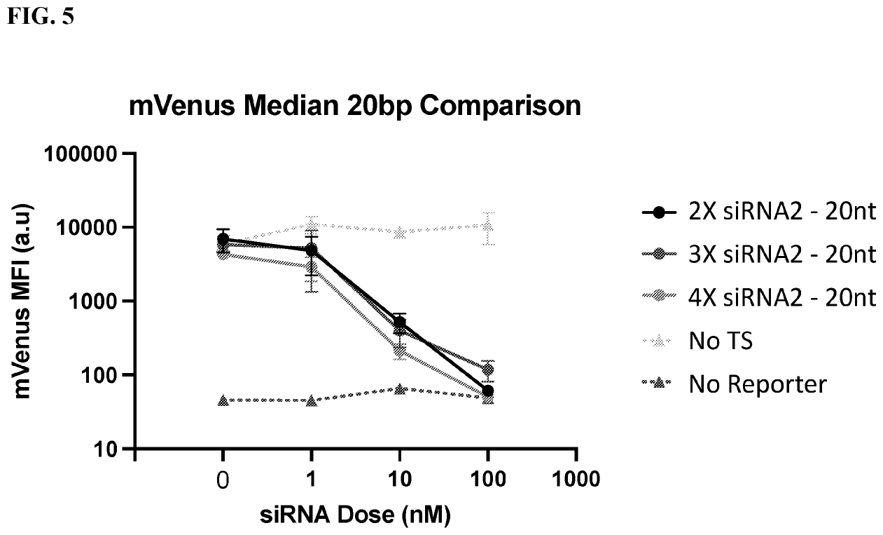

The effect of target site spacing (2-20 nucleotide) on repression, as assessed by mVenus median fluorescence (a.u.) for constrcuts containing either 2X siRNA 2-20nt, 3X siRNA 2-20nt, 4X siRNA 2-20nt, no TS, or No Reporter following administration of siRNA (0, 1, 10, or 100 nM) was determined (FIG. 5). Data represent the median collected via flow cytometry from three technical replicates (n=3) at 20 hours post electroporation of reporter and siRNA. As shown in FIG. 5, modRNA constructs containing 2X-4X siRNA 2 sites with 20nt spacers between target sites placed immediately following the stop codon of an mVenus-PEST reporter demonstrate preferential knockdown in the HEK293T cells with increased co administered doses of siRNA 2. Constructs with 4X siRNA 2 target sites and 20nt has a slightly lower expression than constructs with 2X-20nt or 3X-20nt; however, this is not likely from increased down regulation rather a decrease in maximum expression. The trend holds for low, medium or high dose of siRNA.

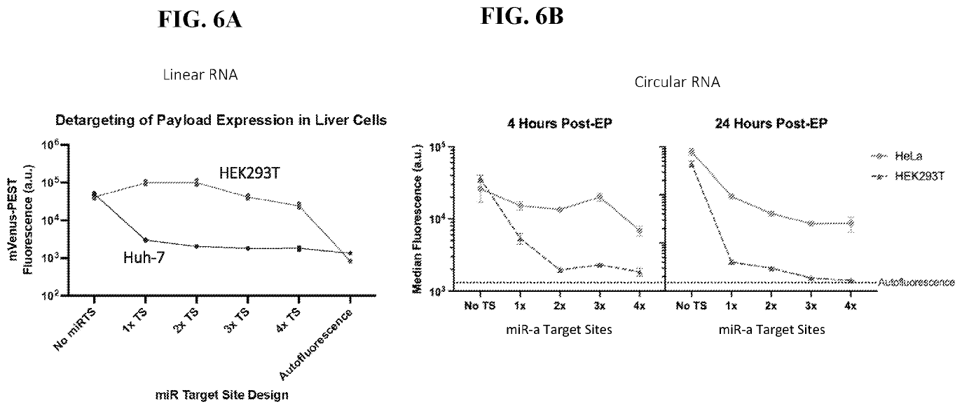

miRNA sensor optimization on various RNA modalities. Then, detargeting of payload expression for linear (FIG. 6A) and circular (FIG. 6B) RNA modalities was assessed. FIG. 6A shows linear modRNA circuits containing an mVenus-PEST reporter and Nx miR-b target sites in the 3′ UTR (1-4X TS) demonstrate preferential knockdown in hepatocytes (Huh-7) vs. a control (HEK293T) cell line (FIG. 6A). This detargeting of payload expression is liver cells is consistent with miR-b being a liver-specific mRNA. Data represent the average geometric mean collected from three technical replicates (n=3) at 20 hours post electroporation. FIG. 6B shows that circRNA constructs containing 1-4 miR-a target sites immediately following the stop codon of an mVenus-PEST reporter demonstrate preferential knockdown in the HEK293T cell line relative to the HeLa cell line. This is consistent with miR-a being highly expressed in HEK293 cells but not in HeLa cells. Incorporation of >2 identical, adjacent target sites does not significantly increase repression. The extent of repression did increase over time, approaching the baseline in HEK293T cells by 24 hours. Data represent the average geometric mean collected from three technical replicates (n 3) at 4 and 24 hours post electroporation.

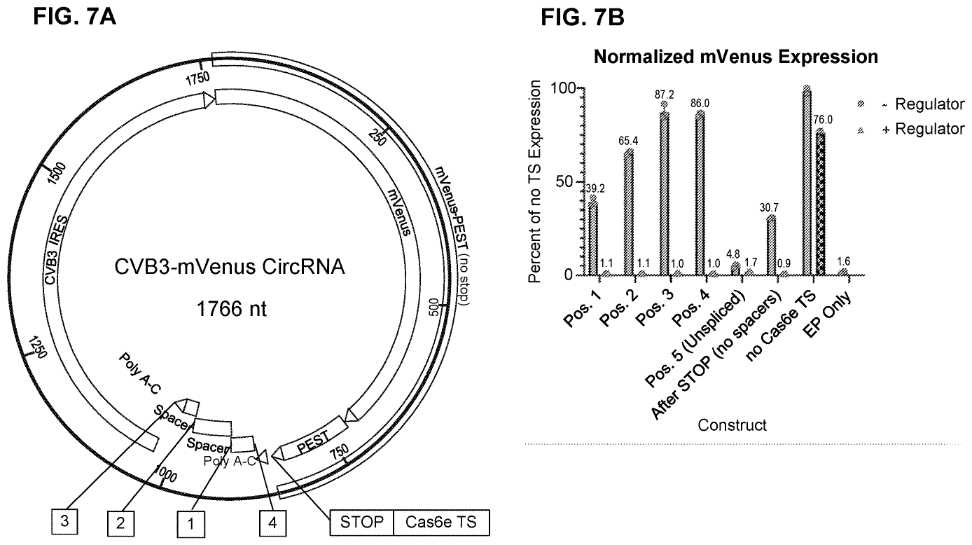

Cas6e target site location optimization. Included here is an example demonstrating that the position of target sites in circRNA determines expression level in the absence of a matching regulator. Specifically, BHK-21 cells were transfected with circRNA expressing mVenus-PEST containing either no Cas6e target site or a Cas6e target site in one of five positions, as indicated in FIG. 7A, as well as modRNA expressing the Cas6e regulator. While expression in the absence of a regulator varied depending on the position of the target site, all constructs saw complete knockdown in the presence of the regulator (FIG. 7B).

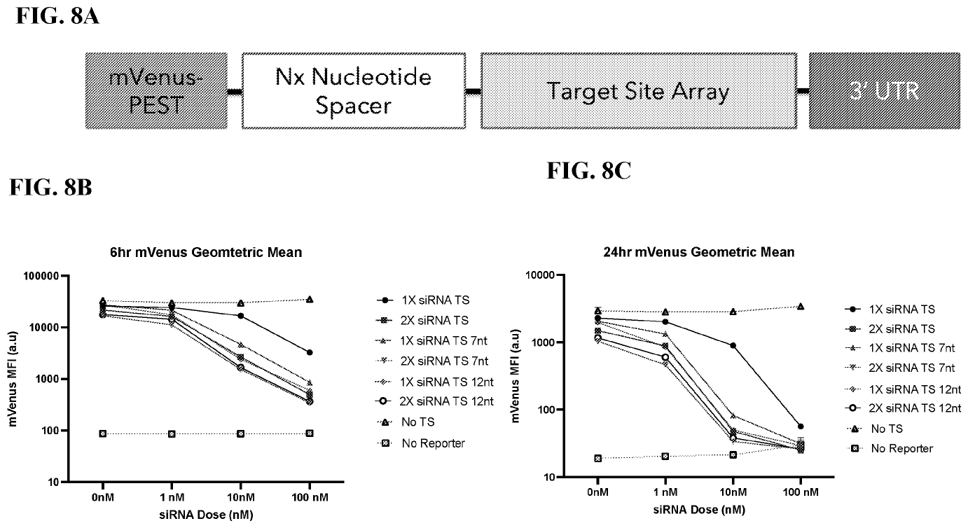

Optimizing the spacing between stop codons and target sites. FIGS. 8B-8C show the effect of Nx Nucleotide spacers depicted in FIG. 8A. Specifically, the FIGS. 8B-8C provide graphs depicting mVenus fluorescence (a.u.) for constructs containing either 1X or 2X siRNA Target Sites (TS), and either 0, 7, 12 nucleotide spacer following administration of siRNA (0, 1, 10, 100 nM). Data represent the Geometric Mean collected via flow cytometry from three technical replicates (n=3) at 6 and 24 hr hours post electroporation of reporter and siRNA.

### Example 2: Multi-Sensor Repression

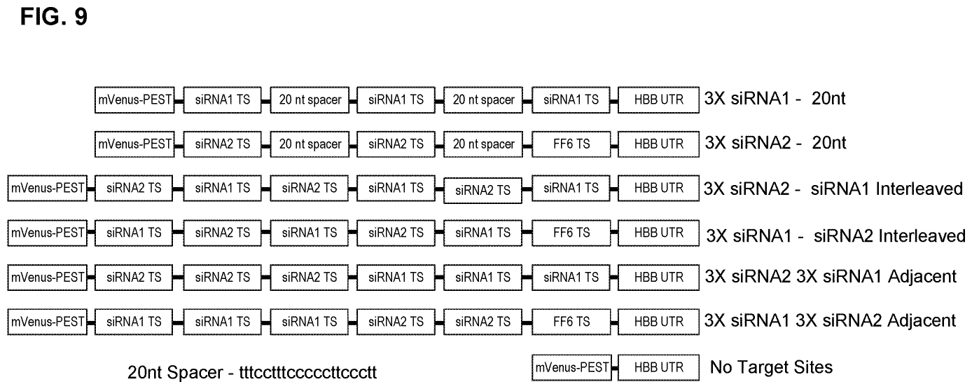

Next, target site arrays for multiple input classifiers, as shown in FIG. 9 were tested for optimal repression.

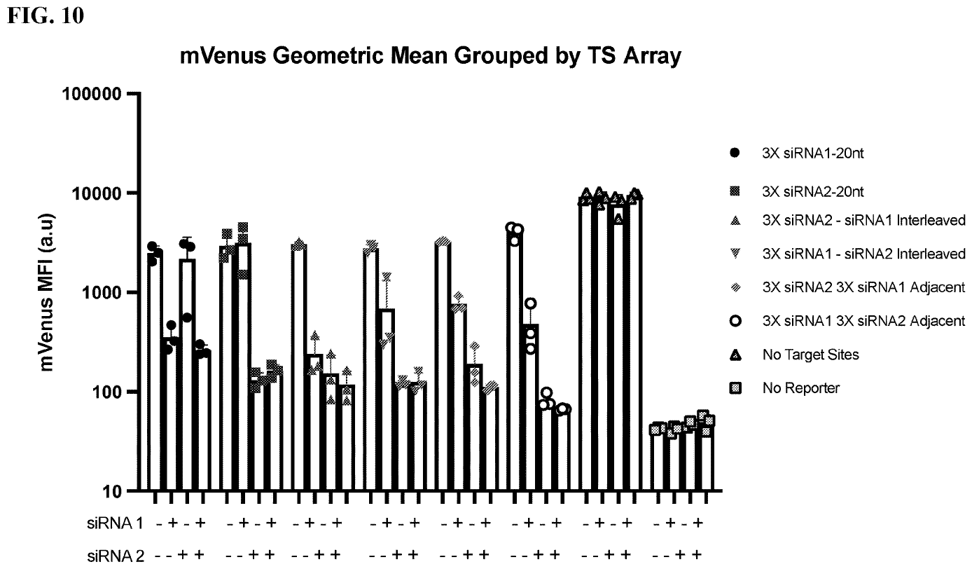

As shown in FIG. 10, HEK293T cells were electroporated with an mVenus-PEST reporter containing a target site array immediately following the stop codon and either, no siRNA, siRNA 1, siRNA 2, or both. Knockdown is observed for all tested constructs (3X siRNA1-20nt, 3X siRNA2-20nt, 3X siRNA2-siRNA1 Interleaved, 3X siRNA1-siRNA2 Interleaved, 3X siRNA2 3X siRNA1 Adjacent, 3X siRNA1 3X siRNA2 Adjacent, no Target Sites, No Reporter) when the co-electroporated siRNA(s) has target sites in the target site array. Combined sensors respond to both siRNA, with no visible synergistic effect, as siRNA2 on its own decreases expression to background. Data represent the average geometric mean collected via flow cytometry from three technical replicates (n=3) at 20 hours post electroporation.

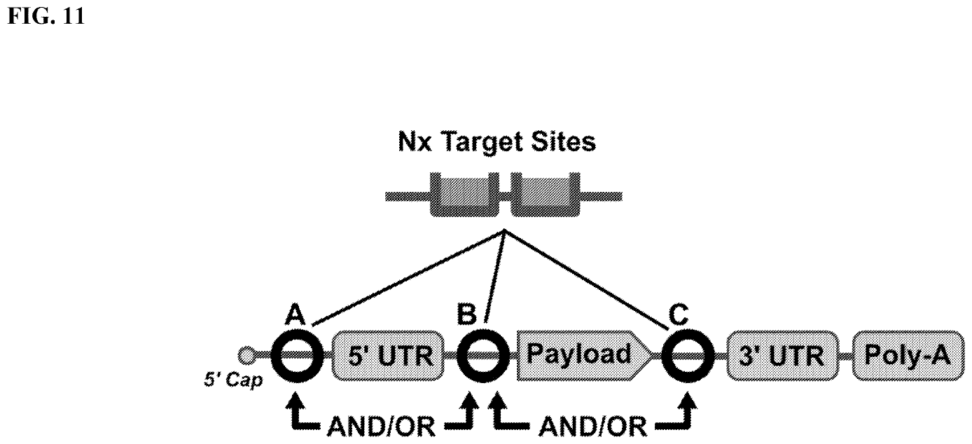

Studies will define the operational range of circuits with Nx miR and regulator target sites placed in either the 5′ or 3′ UTRs in locations A, B, and C outlined in FIG. 11. Studies will specifically assess (1) the type and number of target sites that can be placed in A, B, and C without compromising maximum payload expression in on-target cell types, (2) how the order of target sites in A, B, and C affects payload expression knockdown in different cell types, and (3) the effects of combining miR target sites in both the 5′ and 3′ UTR on payload knockdown.

### Example 3: Synthetic RNA-Based Genetic Circuit Behavior

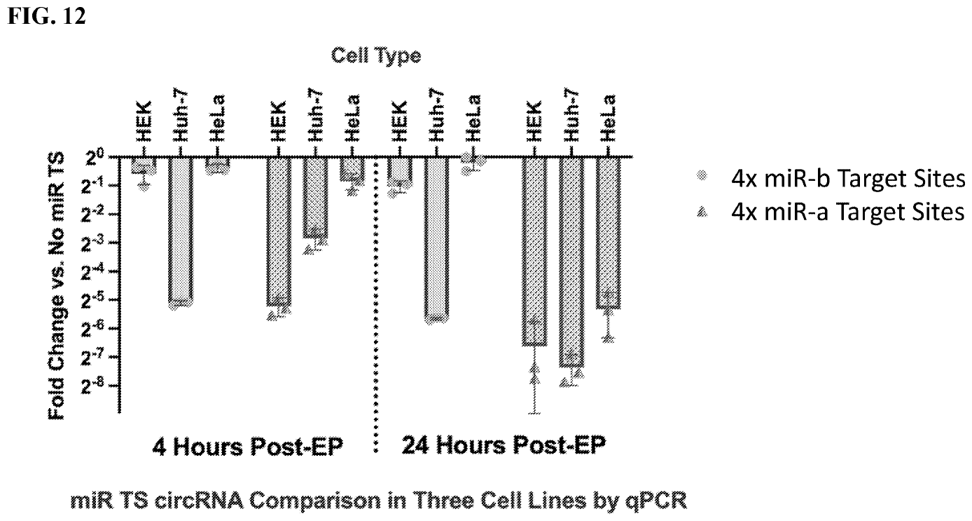

CircRNA with miRNA target sites is degraded by the RISC Complex. As shown in FIG. 12, total RNA was extracted from HEK293T, HeLa, and Huh-7 cells transfected with circular RNA containing either miR-b or miR-a TSs 4- and 24 hours post-transfection. The quantity of transfected circRNA was measured using RT-qPCR with a probe spanning the splice site of the circRNA. HEK cells contain high levels of miR-a, but not miR-b. Huh-7 cells contain high levels of both miR-a and miR-b, and HeLa cells express lesser amounts of miR-a and miR-b. Seen in FIG. 10, circRNA containing target sites for miR-b is significant by 4 hours post transfection in Huh-7 cells while being minimally affected in HEK and HeLa cells. Similarly, circRNA containing target sites for miR-a is downregulated in all cell types at a rate consistent with their relative levels of expression, happening most rapidly in HEK cells followed by Huh-7 and HeLa. circRNA containing no miR sensors was not targeted for degradation and is used as the control for circRNA containing miR sensors.

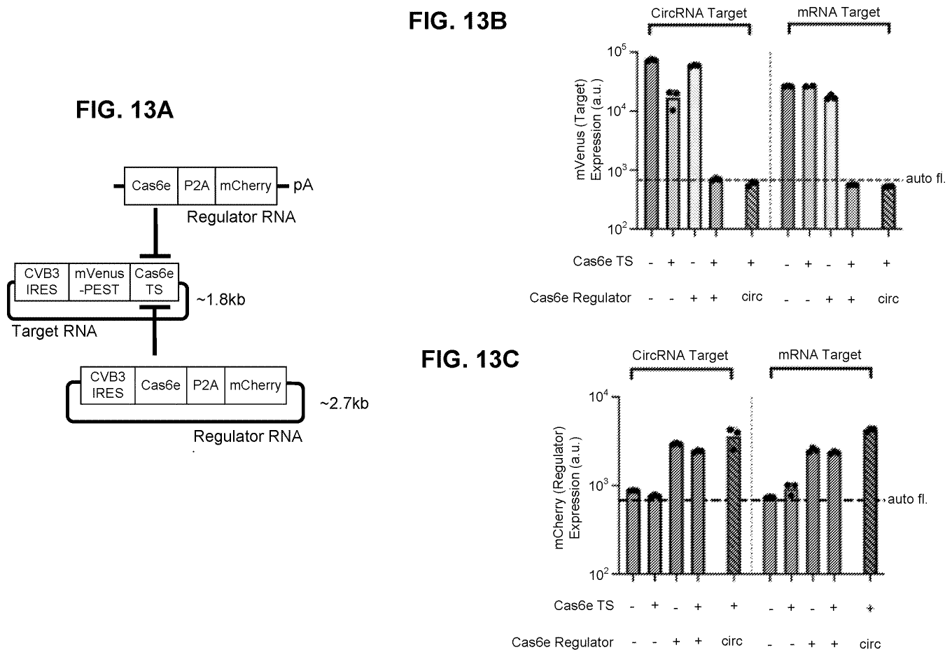

Downregulation of circRNA by Cas6e. The regulatory protein Cas6e efficiently downregulates expression of circRNA molecules containing a protein coding sequence followed by a Cas6e Target Site. BHK-21 cells were transfected with either circular or linear RNA encoding the fluorescent protein mVenus-PEST, each of which either contained no Cas6e target site or a Cas6e target site following the stop codon. Other cells were additionally co-transfected with either a modified linear mRNA or a circRNA encoding the Cas6e regulator, a P2A self-cleaving peptide sequence, and the fluorescent protein mCherry-PEST. See FIG. 13A for a schematic showing linear and circular regulator RNA downregulating target circRNA containing a Cas6e TS and encoding the fluorescent protein mVenus. The downregulation of circRNA containing the target site in the presence of the Cas6e regulator is consistent with results observed for linear mRNA. When both the target site and regulator are present, mVenus expression is reduced to background levels (FIG. 13B). Cas6e has no effect on the expression of circRNA that does not contain its target site (FIG. 13C).

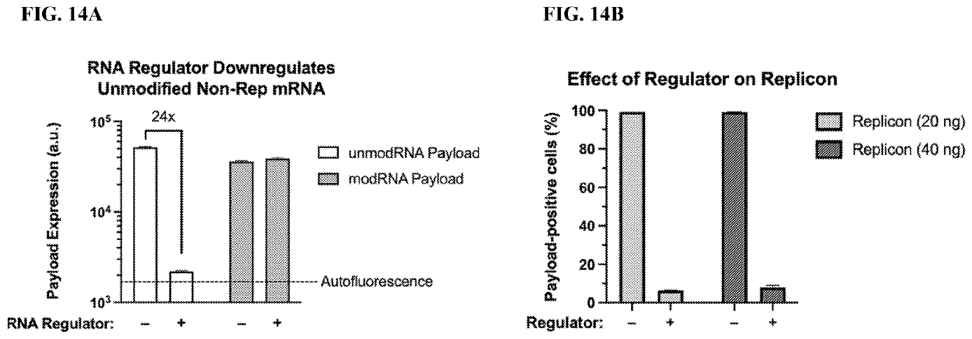

RNA regulator targeting rep and non-rep payloads. In this experiment, an endoribonuclease was utilized as an RNA regulator to control an mRNA strand expressing an mVenus fluorescent reporter payload protein. As shown in in FIG. 14A, linear non-replicating (non-rep) payload mRNA strands bearing a target sequence for the RNA regulator were synthesized either from unmodified bases (unmodRNA Payload) or from bases in which N1-methylpseudouridine was substituted for uridine (modRNA Payload). Payload mRNAs were transfected into BHK-21 cells with or without co-transfection of modRNA expressing the RNA regulator. Cells were assayed by flow cytometry 24 hours later to evaluate payload expression. The RNA regulator downregulated the payload mRNAs synthesized from unmodified bases but did not downregulate the modRNA payload. Further, as shown in FIG. 14B, replicon RNA bearing the RNA regulator's target sequence was transfected at two different doses (20ng or 40ng) into BHK-21 cells with or without co-transfection of modRNA expressing the RNA regulator. While nearly all cells transfected with the replicon alone expressed the payload, co-transfection of the replicon with the RNA regulator reduced the percentage of payload-positive cells to <10%.

### Example 4. Expression of Circular RNA Bearing Homology for Cell-Type-Specific Mirnas is Downregulated in Those Cell Types

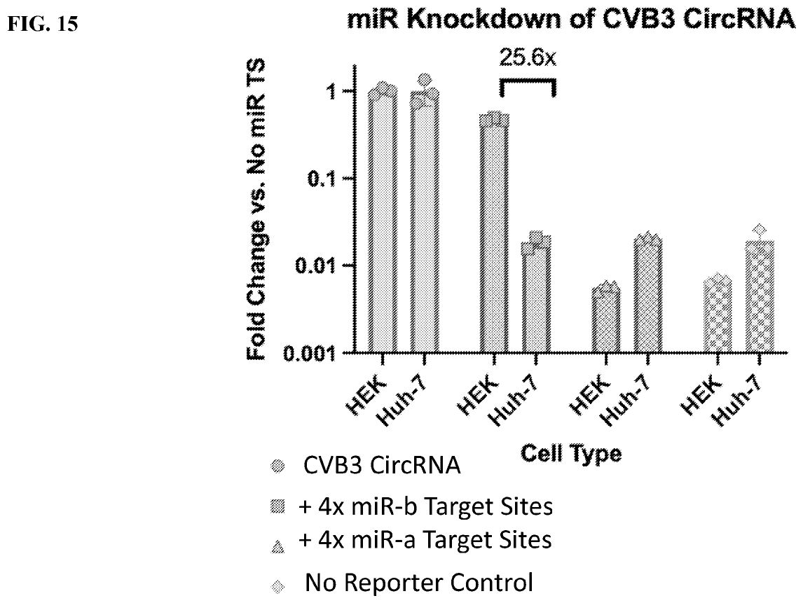

In this example, HEK293T and Huh-7 cells were electroporated with circular RNA encoding EGFP-PEST driven by the coxsackievirus B3 (CVB3) IRES. These circular RNAs contained either no miR TS, 4x miR-b Target Sites, or 4x miR-a Target Sites immediately following the Stop Codon. 24 hours post-transfection, fluorescence of individual cells was measured using flow cytometry, and the data is shown in FIG. 15. In HEK cells, which contain high levels of miR-a but not miR-b, translation of circRNA with 4x miR-a Target Sites was found to be downregulated to autofluorescence levels while circRNA with 4x miR-b Target Sites was not. In Huh-7 cells, which contain levels of both miR-a and miR-b, translation of circRNA with either TS was found to be downregulated to autofluorescence levels. The downregulation of expression of circRNA with miR-b Target Sites in Huh-7 but not HEK293T cells demonstrates that the downregulation is a result of miRNA-mediated RNA degradation.

### Example 5. miRNA Classifiers In Vitro

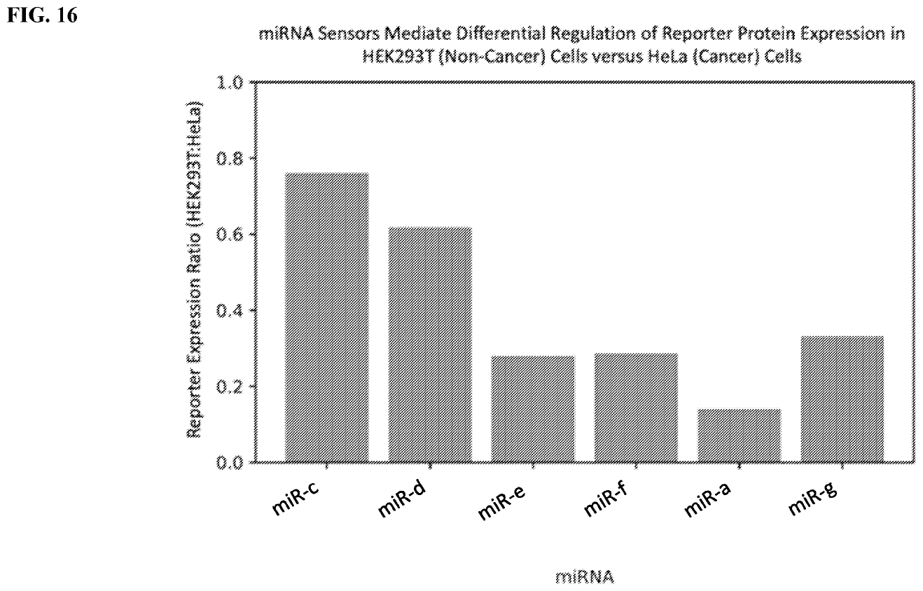

In this Example, non-replicating modRNA that express an mVenus-PEST fluorescent protein and contain various human miRNA target sites, corresponding to miRNAs that have higher activity in HEK293T (non-cancer) cells than HeLa (cancer) cells, were electroporated into both cell types. Flow cytometry data was collected for both cell types (n=3) approximately 24 hours post-electroporation. The geometric mean of mVenus-PEST expression in each cell type was normalized to that of a modRNA with no miRNA sensors, following subtraction of background fluorescence levels. FIG. 16 shows the ratio of these normalized expression levels in HEK293T to those in HeLa.

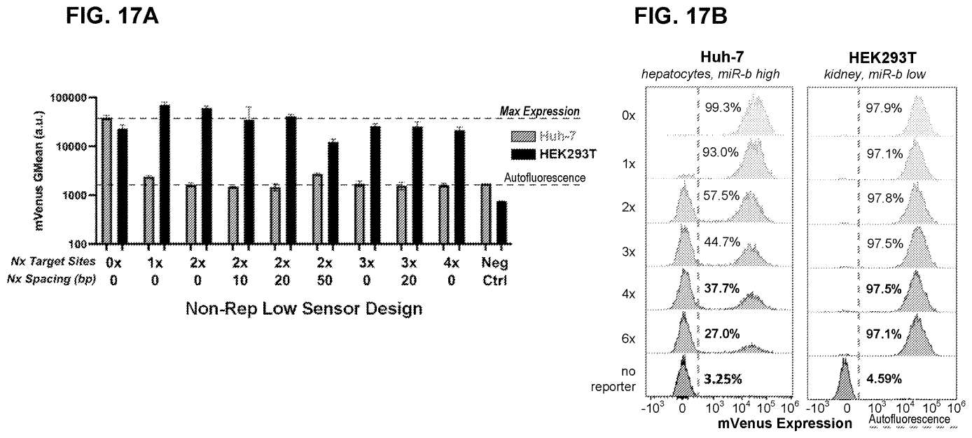

FIGS. 17A-17B shows non-replicating modRNA (FIG. 17A) and replicon RNA (FIG. 17B) reporter constructs containing Nx miR-b target sites in the 3′ UTR were designed and built to express an mVenus-PEST fluorescent protein. Each construct was transfected via electroporation into HEK293T cells or into the human liver cell line Huh-7. A non-replicating modRNA expressing the near-infrared fluorescent reporter protein miRFP720 was co-transfected with each replicon RNA to serve as a transfection marker. Data represents the average geometric mean of three technical replicates (n=3) of flow cytometry data collected approximately 24 hours post-electroporation. The miRFP720-positive population of cells for each cell type (HEK293T or Huh7) was regarded as successfully transfected. The expression output (FIG. 17A) and percentages of miRFP720-positive cells that were also positive for mVenus-PEST (FIG. 17B) were calculated. mVenus-PEST expression was used as a proxy for circuit activity.

While 1x target sites provides near-complete knockdown for non-replicating modRNA, 2x or greater sites yield a complete knockdown reaching autofluorescence levels in the transfected Huh-7 cell line. Additionally, spacing between the miR target sites shows minimal effect on knockdown efficiency (see FIG. 17A).

Nearly all cells containing>0x target sites show mVenus-PEST reporter knockdown in Huh-7 cells which express high levels of miR-b, and no amount of miR-b target sites causes knockdown in miR-b non-expressing HEK293T cells (see FIG. 17B).

Thus, miRNA sensors yield efficient knockdown in vitro.

### Example 6. In Vivo miRNA Sensing

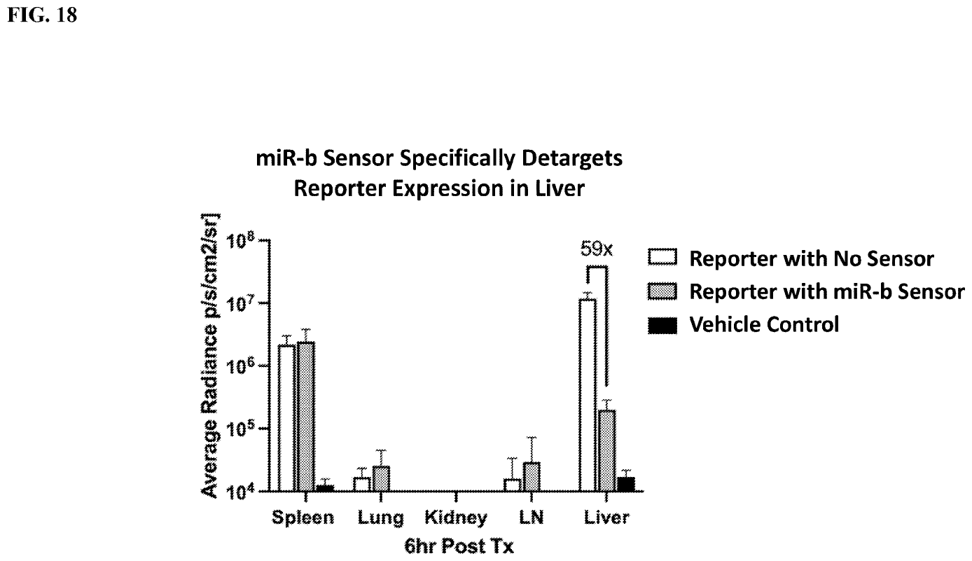

Liver Detargeting. In this Example, mice were injected with lipid nanoparticles bearing reporter modRNA encoding firefly luciferase or with a vehicle control. Mice were sacrificed after 6 hours, and their organs (i.e., spleen, lung, kindey, lymph nodes, and liver) were assessed for luciferase activity. The addition of a sensor for the liver-specific microRNA miR-b to the reporter modRNA resulted in a 59-fold reduction in luciferase expression in the liver compared to the reporter modRNA lacking the sensor. Whereas, luciferase expression in the spleen, lung, kidney, and lymph nodes was not significantly affected by addition of the miR-b sensor. Thus, the miR-b sensor specifically detargets reporter expression in liver (see, FIG. 18).

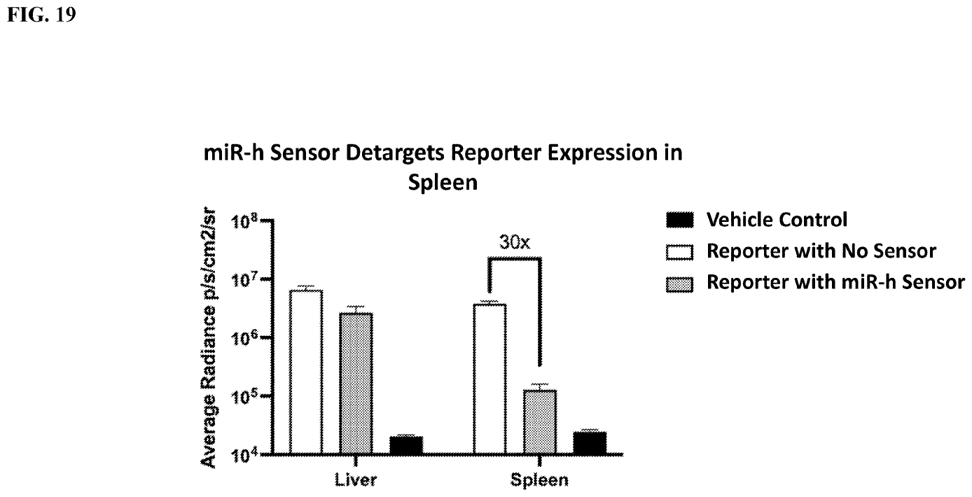

Spleen Detargeting. In this Example, mice were injected with lipid nanoparticles bearing reporter modRNA encoding firefly luciferase or with a vehicle control. Mice were sacrificed after 6 hours, and their organs (i.e., liver and spleen) were assessed for luciferase activity. The addition of a sensor for a spleen-associated miRNA, miR-h, to the reporter modRNA resulted in a 30-fold reduction in luciferase expression in the spleen compared to reporter modRNA lacking the sensor. Meanwhile, addition of the miR-h sensor had only a minimal effect on luciferase expression in the liver. Thus, the miR-h sensor detargets reporter expression in spleen (see, FIG. 19).

### Example 7. Type R Sensors

Type R Sensors Enable Payload Expression when their Cognate Marker is Abundant. In this Example, Huh7 cells, which express high levels of miR-b, and HEK293T cells, which are low in miR-b, were transfected via electroporation with an RNA circuit. The RNA circuit consisted of (1) a replicon payload strand expressing a green fluorescent reporter and comprising a first type P sensor responsive to a regulator protein, Cas6e, and (2) a linear non-replicating regulator strand expressing the regulator protein Cas6e and comprising a type R sensor responsive to miR-b.

As a control, Huh7 and HEK293T cells were transfected in parallel with an otherwise identical RNA circuit that lacks a type R sensor. Expression of the green fluorescent payload was measured via quantitative imaging.

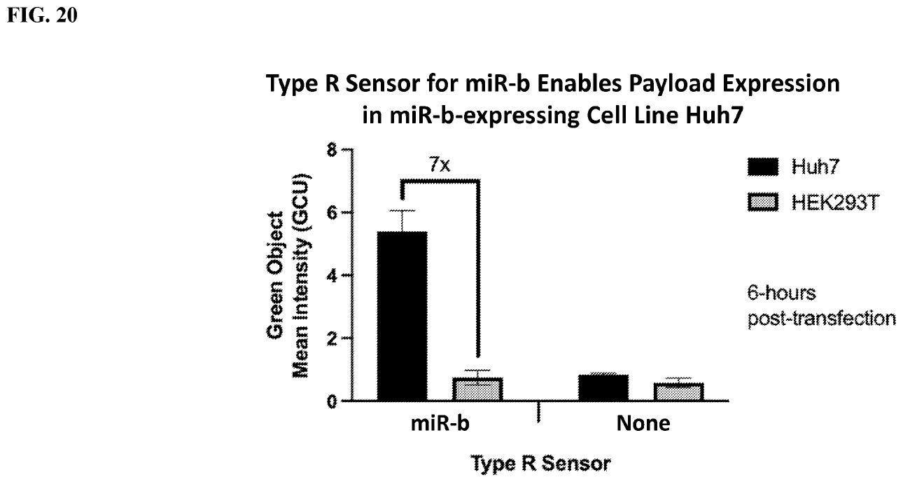

At 6 hours post-transfection, payload expression from the RNA circuit with the miR-b type R sensor turns on in the miR-b-rich Huh7 cells, while payload expression remains OFF in HEK293T cells—in fact, there is a 7× reduction in payload expression in HEK293T cells compared to Huh7 cells (see, FIG. 20). Payload expression from the RNA circuit lacking a type R sensor remains OFF in either cell type (see, FIG. 20).

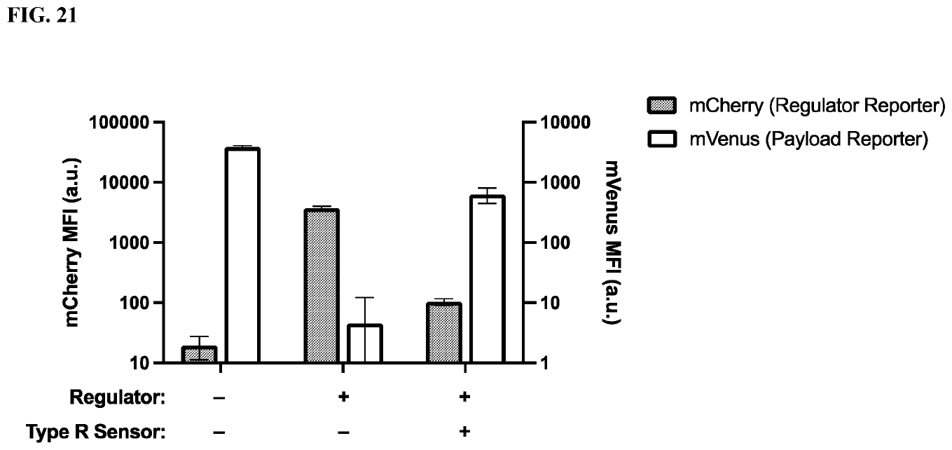

Type R Sensors Enable Payload Expression when their Cognate Marker is Abundant. A549 lung cancer cells, which express high levels of miR-i, were transfected via electroporation with a replicon payload sequence expressing an mVenus reporter and comprising a first type P sensor responsive to the Cas6e regulator protein. Some cells were co-transfected with a linear non-replicating regulator sequence expressing the Cas6e regulator protein linked to an mCherry reporter via a 2A self-cleaving peptide. This resulted in suppression of the payload sequence, as evidenced by reduction of mVenus expression. However, some cells were co-transfected with a version of the regulator sequence comprising a type R sensor for miR-i. In these cells, the type R sensor is activated, resulting in downregulation of the regulator strand as indicated by suppression of mCherry, and enabling expression of the payload sequence, as evidenced by a significant increase in mVenus expression (see, FIG. 21).

### Example 8. Effector Testing on a modRNA Payload

The effectiveness of a linear non-replicating regulator sequence containing a human Pum1 (PUF) RNA binding domain (RBD) and various effector domains [i.e., cNOT7, TTP, DDX6, MCPIP1PIN, Dis3PIN (isoform 2), and SMG6PIN (isoform 2)] to inhibit expression of a modified mRNA (modRNA) payload was assessed. The RBD of human PUF recognizes the following 8nt target RNA sequence, 5′-UGGAUGAA-3′ (i.e., PUF TS #1; PUF_{UGG} binding site). First, the mRNA circuitry was transfected into cells using electroporation then fluorescent cells were visualized every 2 hours for 30 hours to assess mVenus expression.

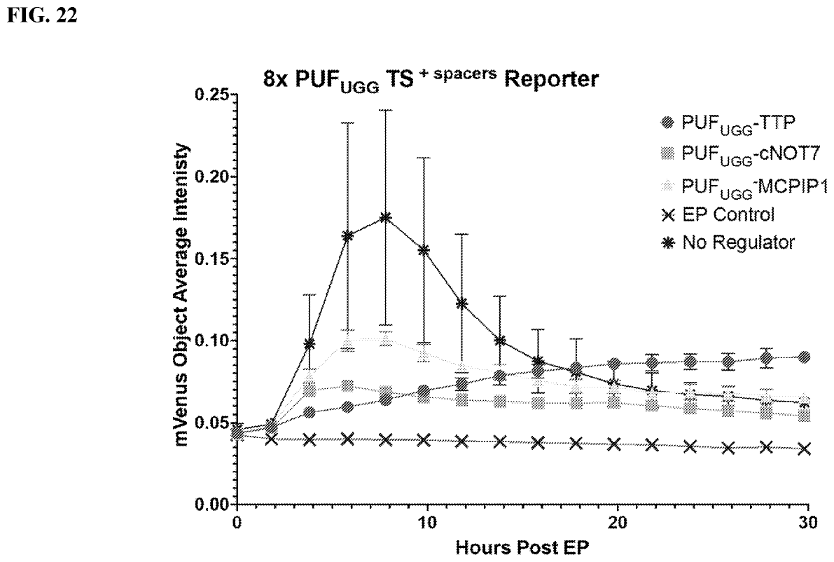

FIG. 22 shows expression of the modRNA payload 0-30 hours post electroporation. Effectors cNOT7, TTP, and MCPIP1_{PIN} downregulate payload expression when the payload sequence contained 8x TS (FIG. 22).

All payload constructs had unique, randomly generated 12 base pair spacer sequences before the first PUF TS, between every PUF TS, and after the final PUF TS.

### Example 9. Effector Testing on a repRNA Payload

The effectiveness of a linear non-replicating regulator sequence containing a human PUF RBD and the tested effector domains (i.e., cNOT7, TTP, DDX6, and MCPIP1_{PIN}) to inhibit expression of a self-amplifying replicon RNA (repRNA) payload was assessed. The RBD of human PUF recognizes the following 8nt target RNA sequence, 5′-UGGAUGAA-3′ (i.e., TS #1; PUF_{UGG} binding site). First, the mRNA circuitry was transfected into cells using electroporation then fluorescent cells were visualized every 2 hours for 200 hours to assess mVenus reporter activity.

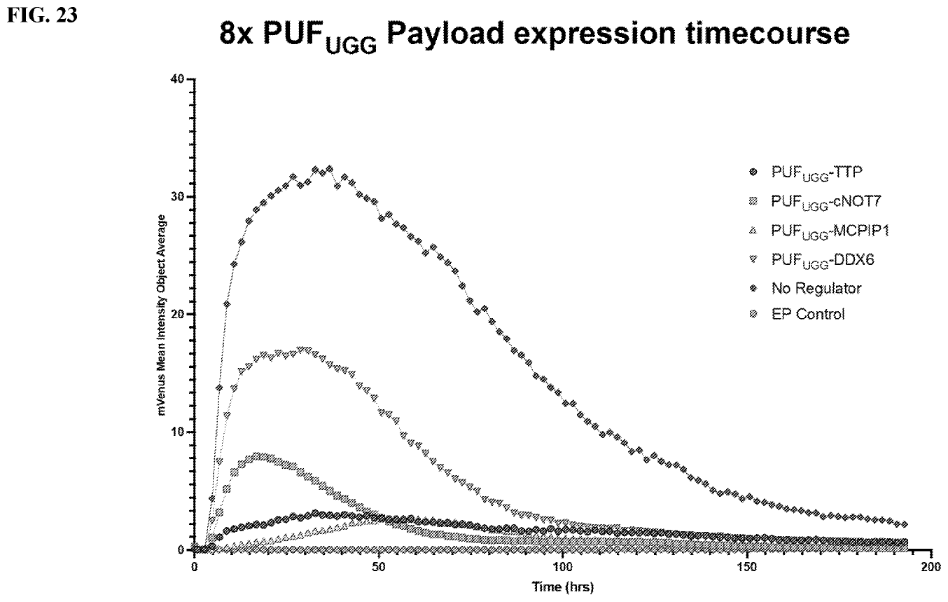

FIG. 23 shows the expression of the payload 0-200 hours post electroporation, demonstrating that effectors cNOT7, TTP, and MCPIP1_{PIN} downregulated payload expression when the payload sequence contained 8x PUF TS. All payload constructs had unique, randomly generated 12 base pair spacer sequences before the first PUF TS, between every PUF TS, and after the final PUF TS.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A synthetic circuit comprising:
(a) a first nucleotide sequence encoding a payload (payload sequence) and
(b) a second nucleotide sequence encoding a regulator (regulator sequence),
wherein the payload sequence comprises a sensor that is capable of specifically recognizing the regulator (type P sensor),
wherein the regulator sequence comprises a sensor that is capable of specifically recognizing a marker (type R sensor), and
wherein the regulator and the marker recognized by the type R sensor (type R marker) are not the same.

2. The synthetic circuit of claim 1, wherein the payload sequence comprises a plurality of the type P sensor.

3. The synthetic circuit of claim 2, wherein the plurality of the type P sensor comprises two type P sensors, three type P sensors, four type P sensors, five type P sensors, six type P sensors, seven type P sensors, or eight or more type P sensors.

4. The synthetic circuit of claim 2 or 3, wherein each of the type P sensors is the same.

5. The synthetic circuit of claim 2 or 3, wherein one or more of the type P sensors are different.

6. The synthetic circuit of any one of claims 1 to 5, wherein the payload sequence comprises a spacer sequence (type P spacer).

7. The synthetic circuit of claim 6, wherein the payload sequence comprises a plurality of type P spacer.

8. The synthetic circuit of claim 7, wherein each of the type P spacers is the same.

9. The synthetic circuit of claim 7, wherein one or more of the type P spacers are different.

10. The synthetic circuit of any one of claims 6 to 9, wherein: (a) at least one type P spacer is positioned upstream of the type P sensor, (b) at least one type P spacer is positioned downstream of the type P sensor, or (c) both (a) and (b).

11. The synthetic circuit of any one of claims 6 to 10, which comprises at least two type P sensors, wherein at least one type P spacer is positioned between the at least two type P sensors.

12. A synthetic circuit comprising:
(a) a first nucleotide sequence encoding a payload (payload sequence) and
(b) a second nucleotide sequence encoding a regulator (regulator sequence),
wherein the payload sequence comprises a first sensor that is capable of specifically recognizing the regulator (first type P sensor) and a second sensor that is capable of specifically recognizing a marker (second type P sensor),
wherein the regulator sequence comprises a sensor that is capable of specifically recognizing a marker (type R sensor), and
wherein the regulator, the marker recognized by the second type P sensor (second type P marker), and/or the marker recognized by the type R sensor (type R marker) are not the same.

13. The synthetic circuit of claim 12, wherein the payload sequence comprises a plurality of the first type P sensor.

14. The synthetic circuit of claim 13, wherein the plurality of the first type P sensor comprises two first type P sensors, three first type P sensors, four first type P sensors, five first type P sensors, six first type P sensors, seven first type P sensors, eight first type P sensors, nine first type P sensors, ten first type P sensors, eleven first type P sensors, or twelve first type P sensors.

15. The synthetic circuit of claim 13 or 14, wherein each of the first type P sensors is the same.

16. The synthetic circuit of claim 13 or 14, wherein one or more of the first type P sensors are different.

17. The synthetic circuit of any one of claims 12 to 16, wherein the payload sequence comprises a plurality of the second type P sensor.

18. The synthetic circuit of claim 17, wherein the plurality of the second type P sensor comprises two second type P sensors, three second type P sensors, four second type P sensors, five second type P sensors, six second type P sensors, seven second type P sensors, eight second type P sensors, nine second type P sensors, ten second type P sensors, eleven second type P sensors, or twelve second type P sensors.

19. The synthetic circuit of claim 17 or 18, wherein each of the second type P sensors is the same.

20. The synthetic circuit of claim 17 or 18, wherein one or more of the second type P sensors are different.

21. The synthetic circuit of any one of claims 12 to 20, wherein the payload sequence comprises a spacer sequence (type P spacer).

22. The synthetic circuit of claim 21, wherein the payload sequence comprises a plurality of type P spacer.

23. The synthetic circuit of claim 22, wherein each of the type P spacers is the same.

24. The synthetic circuit of claim 22, wherein one or more of the type P spacers are different.

25. The synthetic circuit of any one of claims 22 to 24, wherein: (a) at least one type P spacer is positioned between the first type P sensor and the second type P sensor; (b) at least one type P spacer is positioned upstream of both the first type P sensor and the second type P sensor; (c) at least one type P spacer is positioned downstream of both the first type P sensor and the second type P sensor; or (d) any combination of (a) to (c).

26. The synthetic circuit of any one of claims 22 to 24, which comprises the plurality of the first type P sensor, wherein two or more of the first type P sensors are separated by a type P spacer.

27. The synthetic circuit of claim 26, wherein each of the first type P sensors are separated by a type P spacer.

28. The synthetic circuit of any one of claims 21 to 27, which comprises the plurality of the second type P sensor, wherein two or more of the second type P sensors are separated by a type P spacer.

29. The synthetic circuit of claim 28, wherein each of the second type P sensors are separated by a type P spacer.

30. The synthetic circuit of any one of claims 21 to 29, wherein the type P spacer is between about 1 to about 50 nucleotides in length.

31. The synthetic circuit of claim 30, wherein the type P spacer is at least about 10 nucleotides in length.

32. The synthetic circuit of claim 30, wherein the type P spacer is about 10 nucleotides in length, about 20 nucleotides in length, or about 50 nucleotides in length.

33. The synthetic circuit of any one of claims 21 to 32, wherein the type P spacer comprises, consists essentially of, or consists of the sequence tttcctttcccccttccctttttcctttcctttcctttcccccttccctt (SEQ ID NO: 1), tttcctttcccccttccctt (SEQ ID NO: 2) or gcggccgctaaa (SEQ ID NO: 3) or fragments thereof.

34. The synthetic circuit of any one of claims 1 to 33, wherein the regulator sequence comprises a plurality of the type R sensor.

35. The synthetic circuit of claim 34, wherein the plurality of the type R sensor comprises two type R sensors, three type R sensors, four type R sensors, five type R sensors, six type R sensors, seven type R sensors, or eight or more type R sensors.

36. The synthetic circuit of claim 34 or 35, wherein each of the type R sensors is the same.

37. The synthetic circuit of claim 34 or 35, wherein one or more of the type R sensors are different.

38. The synthetic circuit of any one of claims 1 to 37, wherein the regulator sequence comprises a spacer sequence (type R spacer).

39. The synthetic circuit of claim 38, wherein the regulator sequence comprises a plurality of type R spacer.

40. The synthetic circuit of claim 39, wherein each of the type R spacer is the same.

41. The synthetic circuit of claim 39, wherein one or more of the type R spacers are different.

42. The synthetic circuit of any one of claims 38 to 41, which comprises the plurality of the type R sensor, wherein two or more of the type R sensors are separated by a type R spacer.

43. The synthetic circuit of claim 42, wherein each of the type R sensors are separated by a type R spacer.

44. The synthetic circuit of any one of claims 38 to 43, wherein at least one type R spacer is upstream of at least one type R sensor.

45. The synthetic circuit of any one of claims 38 to 44, wherein the type R spacer is between about 1 to about 50 nucleotides in length.

46. The synthetic circuit of claim 45, wherein the type R spacer is at least about 10 nucleotides in length.

47. The synthetic circuit of claim 45, wherein the type R spacer is about 10 nucleotides in length, about 20 nucleotides in length, or about 50 nucleotides in length.

48. The synthetic circuit of any one of claims 38 to 47, wherein the type R spacer comprises, consists essentially of, or consists of the sequence tttcctttcccccttccctttttcctttcctttcctttcccccttccctt (SEQ ID NO: 1) or a fragment thereof.

49. The synthetic circuit of claim 48, wherein the type R spacer comprises, consists essentially of, or consists of the sequence tttcctttcccccttccctt (SEQ ID NO: 2).

50. The synthetic circuit of claims 38 to 45, wherein the type R spacer comprises, consists essentially of, or consists of the sequence gcggccgctaaa (SEQ ID NO: 3).

51. The synthetic circuit of any one of claims 1 to 50, wherein the first marker, the second marker, or the first and second markers comprise a microRNA, a protein, a metabolite, or combinations thereof.

52. The synthetic circuit of any one of claims 1 to 51, wherein the regulator comprises a RNA-binding protein, siRNA, shRNA, pre-miRNA, ribozyme, or combinations thereof.

53. The synthetic circuit of claim 52, wherein the RNA-binding protein comprises a ribonuclease.

54. The synthetic circuit of claim 53, wherein the ribonuclease comprises a Cas protein.

55. The synthetic circuit of claim 54, wherein the Cas protein comprises a Cas6 protein.

56. The synthetic circuit of any one of claims 1 to 55, wherein the payload sequence, the regulator sequence, or both the payload and regulator sequences comprise a linear RNA or a circular RNA.

57. The synthetic circuit of any one of claims 1 to 56, wherein the payload sequence is a self-replicating RNA and the regulator sequence is a non-replicating RNA.

58. The synthetic circuit of claim 56 or 57, wherein the payload sequence is a self-replicating RNA and the regulator sequence is a circular RNA.

59. The synthetic circuit of claim 56 or 57, wherein the payload sequence is a self-replicating RNA and the regulator sequence is a linear non-replicating RNA.

60. The synthetic circuit of claim 56 or 57, wherein the payload sequence is a circular RNA and the regulator sequence is a circular RNA.

61. The synthetic circuit of claim 56 or 57, wherein the payload sequence is a circular RNA and the regulator sequence is a linear non-replicating RNA.

62. A synthetic circuit comprising:
(a) a first nucleotide sequence encoding a payload (payload sequence) and
(b) a second nucleotide sequence encoding a regulator (regulator sequence);
wherein the payload sequence is a self-replicating RNA and comprises a sensor that is capable of specifically recognizing the regulator (type P sensor);
wherein the regulator sequence is a circular RNA and comprises a sensor that is capable of specifically recognizing a marker (type R sensor); and
wherein the regulator and the marker recognized by the type R sensor (type R marker) are not the same.

63. A synthetic circuit comprising: (a) a first nucleotide sequence encoding a payload (payload sequence) and (b) a second nucleotide sequence encoding a regulator (regulator sequence);
wherein the payload sequence is a self-replicating RNA and comprises a first sensor that is capable of specifically recognizing the regulator (first type P sensor) and a second sensor that is capable of specifically recognizing a marker (second type P sensor);
wherein the regulator sequence is a circular RNA and comprises a sensor that is capable of specifically recognizing a marker (type R sensor); and
wherein the regulator, the marker recognized by the second type P sensor (type P marker), and the marker recognized by the type R sensor (type R marker) are not the same.

64. A synthetic circuit comprising:
(a) a first nucleotide sequence encoding a payload (payload sequence) and
(b) a second nucleotide sequence encoding a regulator (regulator sequence);
wherein the payload sequence is a self-replicating RNA comprises a sensor that is capable of specifically recognizing the regulator (type P sensor);
wherein the regulator sequence is a non-replicating linear RNA and comprises a sensor that is capable of specifically recognizing a marker (type R sensor); and
wherein the regulator and the marker recognized by the type R sensor (type R marker) are not the same.

65. A synthetic circuit comprising: (a) a first nucleotide sequence encoding a payload (payload sequence) and (b) a second nucleotide sequence encoding a regulator (regulator sequence);
wherein the payload sequence is a self-replicating RNA and comprises a first sensor that is capable of specifically recognizing the regulator (first type P sensor) and a second sensor that is capable of specifically recognizing a marker (second type P sensor);
wherein the regulator sequence is a non-replicating linear RNA and comprises a sensor that is capable of specifically recognizing a marker (type R sensor); and
wherein the regulator, the marker recognized by the second type P sensor (type P marker), and the marker recognized by the type R sensor (type R marker) are not the same.

66. A synthetic circuit comprising:
(a) a first nucleotide sequence encoding a payload (payload sequence) and
(b) a second nucleotide sequence encoding a regulator (regulator sequence);
wherein the payload sequence is a circular RNA and comprises a sensor that is capable of specifically recognizing the regulator (type P sensor);
wherein the regulator sequence is a circular RNA and comprises a sensor that is capable of specifically recognizing a marker (type R sensor); and
wherein the regulator and the marker recognized by the type R sensor (type R marker) are not the same.

67. A synthetic circuit comprising: (a) a first nucleotide sequence encoding a payload (payload sequence) and (b) a second nucleotide sequence encoding a regulator (regulator sequence);
wherein the payload sequence is a circular RNA and comprises a first sensor that is capable of specifically recognizing the regulator (first type P sensor) and a second sensor that is capable of specifically recognizing a marker (second type P sensor);
wherein the regulator sequence is a circular RNA and comprises a sensor that is capable of specifically recognizing a marker (type R sensor); and
wherein the regulator, the marker recognized by the second type P sensor (type P marker), and the marker recognized by the type R sensor (type R marker) are not the same.

68. A synthetic circuit comprising:
(a) a first nucleotide sequence encoding a payload (payload sequence) and
(b) a second nucleotide sequence encoding a regulator (regulator sequence);
wherein the payload sequence is a circular RNA and comprises a sensor that is capable of specifically recognizing the regulator (type P sensor);
wherein the regulator sequence is a non-replicating linear RNA and comprises a sensor that is capable of specifically recognizing a marker (type R sensor); and
wherein the regulator and the marker recognized by the type R sensor (type R marker) are not the same.

69. A synthetic circuit comprising: (a) a first nucleotide sequence encoding a payload (payload sequence) and (b) a second nucleotide sequence encoding a regulator (regulator sequence);
wherein the payload sequence is a circular RNA and comprises a first sensor that is capable of specifically recognizing the regulator (first type P sensor) and a second sensor that is capable of specifically recognizing a marker (second type P sensor);
wherein the regulator sequence is a non-replicating linear RNA and comprises a sensor that is capable of specifically recognizing a marker (type R sensor); and
wherein the regulator, the marker recognized by the second type P sensor (type P marker), and the marker recognized by the type R sensor (type R marker) are not the same.

70. The synthetic circuit of any one of claims 62 to 69, wherein:
(a) the payload sequence comprises a plurality of the first type P sensor,
(b) the payload sequence comprises a plurality of the second type P sensor,
(c) the regulator sequence comprises a plurality of the type R sensor, or
(d) any combination of (a) to (c).

71. The synthetic circuit of any one of claims 62 to 70, wherein:
(a) the payload sequence comprises a spacer sequence (type P spacer),
(b) the regulator sequence comprises a spacer sequence (type R spacer), or
(c) both (a) and (b).

72. The synthetic circuit of claim 71, wherein: (a) the type P spacer is positioned between the first type P sensor and the second type P sensor; (b) the type P spacer is positioned upstream of both the first type P sensor and the second type P sensor; (c) the type P spacer is positioned downstream of both the first type P sensor and the second type P sensor; or (d) any combination of (a) to (c).

73. The synthetic circuit of claim 71 or 72, wherein the payload sequence comprises the plurality of the first type P sensor, wherein two or more of the first type sensors are separated by a type P spacer.

74. The synthetic circuit of any one of claims 71 to 73, wherein the payload sequence comprises the plurality of the second type P sensor, wherein two or more of the second type sensors are separated by a type P spacer.

75. The synthetic circuit of any one of claims 71 to 74, wherein the regulator sequence comprises the plurality of the type R sensor, wherein two or more of the type R sensors are separated by a type R spacer.

76. The synthetic circuit of any one of claims 71 to 75, wherein the type P spacer, type R spacer, or both are between about 1 to about 50 nucleotides in length.

77. The synthetic circuit of any one of claims 71 to 76, wherein the type P spacer, type R spacer, or both comprise, consist essentially of, or consist of the sequence tttcctttcccccttccctttttcctttcctttcctttcccccttccctt (SEQ ID NO: 1) or a fragment thereof.

78. The synthetic circuit of claim 77, wherein the type P spacer, type R spacer, or both comprise, consist essentially of, or consist of the sequence tttcctttcccccttccctt (SEQ ID NO: 2).

79. The synthetic circuit of any one of claims 71 to 78, wherein the type P spacer, type R spacer, or both comprise, consist essentially of, or consist of the sequence gcggccgctaaa (SEQ ID NO: 3) or a fragment thereof.

80. The synthetic circuit of claim 79, wherein the type P spacer, type R spacer, or both comprise, consist essentially of, or consist of the sequence gcggccgctaaa (SEQ ID NO: 3).

81. The synthetic circuit of any one of claims 62 to 80, wherein the type P marker, type R marker, or both comprise a microRNA, a protein, a metabolite, or combinations thereof.

82. The synthetic circuit of any one of claims 62 to 81, wherein the regulator comprises a RNA-binding protein, siRNA, shRNA, pre-miRNA, ribozyme, or combinations thereof.

83. The synthetic circuit of claim 82, wherein the RNA-binding protein comprises a ribonuclease.

84. The synthetic circuit of claim 83, wherein the ribonuclease comprises a Cas protein.

85. The synthetic circuit of claim 83, wherein the Cas protein comprises a Cas6 protein.

86. A synthetic circuit comprising:
(a) a first nucleotide sequence encoding a payload (payload sequence) and
(b) a second nucleotide sequence encoding a regulator (regulator sequence),
wherein, when the payload sequence and the regulator sequence are present in a target cell, the payload is expressed in the target cell for a first expression and the regulator is expressed in the target cell for a second expression, and
wherein the first expression is greater than the second expression.

87. The synthetic circuit of claim 86, wherein:
(a) the payload sequence comprises a first sensor that is capable of specifically recognizing the regulator (first type P sensor) and a second sensor that is capable of specifically recognizing a marker (second type P sensor),
(b) the regulator sequence comprises a sensor that is capable of specifically recognizing a marker (type R sensor); and
wherein the regulator, the marker recognized by the second type P sensor (type P marker), and the marker recognized by the type R sensor (type R marker) are not the same.

88. The synthetic circuit of claim 87, wherein the recognition of the type P marker by the second type P sensor inhibits the expression of the payload.

89. The synthetic circuit of any one of claims 86 to 88, wherein the recognition of the type R marker by the type R sensor inhibits the expression of the regulator.

90. The synthetic circuit of any one of claims 87 to 89, wherein: (a) the target cell does not express sufficient levels of the type P marker to turn on the second type P sensor, and (b) the target cell expresses sufficient levels of the type R marker to turn on the type R sensor.

91. The synthetic circuit of any one of claims 87 to 90, wherein: (a) the non-target cell expresses sufficient levels of the type P marker to turn on the second type P sensor, (b) the non-target cell does not express sufficient levels of the type R marker to turn on the type R sensor, or (c) both (a) and (b).

92. A synthetic circuit comprising:
(a) a first nucleotide sequence encoding a payload (payload sequence) and
(b) a second nucleotide sequence encoding a regulator (regulator sequence),
wherein, when the payload sequence and the regulator sequence are present in a non-target cell, the payload is expressed in the non-target cell for a first expression and the regulator is expressed in the non-target cell for a second expression, and
wherein the second expression is greater than the first expression.

93. The synthetic circuit of claim 92, wherein:
(a) the payload sequence comprises a first sensor that is capable of specifically recognizing the regulator (first type P sensor) and a second sensor that is capable of specifically recognizing a marker (second type P sensor), and
(b) the regulator sequence comprises a sensor that is capable of specifically recognizing a marker (type R sensor);
and wherein the regulator, the marker recognized by the second type P sensor (type P marker), and the marker recognized by the type R sensor (type R marker) are not the same.

94. The synthetic circuit of claim 92 or 93, wherein the binding of the type P marker to the second type P sensor inhibits the expression of the payload.

95. The synthetic circuit of claim 92, wherein: the non-target cell comprises (a) sufficient levels of the type P marker to turn on the second type P sensor, (b) levels of the type R marker insufficient to turn on the type R sensor, or (c) both (a) and (b).

96. The synthetic circuit of any one of claims 92 to 95, wherein the binding of the type R marker to the type R sensor inhibits the expression of the regulator.

97. The synthetic circuit of any one of claims 92, 95, and 96, wherein the target cell comprises: (a) levels of the type P marker insufficient to turn on the second type P sensor, and and (b) sufficient levels of the type R marker to turn on the type R sensor.

98. A synthetic circuit comprising:
(a) a first nucleotide sequence encoding a payload (payload sequence) and
(b) a second nucleotide sequence encoding a regulator (regulator sequence),
wherein, when the synthetic circuit is contacted with a population of cells comprising a target cell and a non-target cell, the expression of the payload in the target cell is higher than the corresponding expression in the non-target cell.

99. The synthetic circuit of claim 98, wherein the expression of the payload in the target cell is higher than the corresponding expression in the non-target cell by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to the corresponding expression in the non-target cell.

100. The synthetic circuit of claim 98 or 99, wherein:
(a) the payload sequence comprises a first sensor that is capable of specifically recognizing the regulator (first type P sensor) and a second sensor that is capable of specifically recognizing a marker (second type P sensor), and
(b) the regulator sequence comprises a sensor that is capable of specifically recognizing a marker (type R sensor); and
wherein the regulator, the marker recognized by the second type P sensor (type P marker), and the marker recognized by the type R sensor (type R marker) are not the same.

101. The synthetic circuit of any one of claim 98 to 100, wherein the binding of the type P marker to the second type P sensor inhibits the expression of the payload.

102. The synthetic circuit of any one of claims 98 to 101, wherein the binding of the type R marker to the type R sensor inhibits the expression of the regulator.

103. The synthetic circuit of any one of claims 98 to 102, wherein: (a) the target cell does not comprise sufficient level of the type P marker to turn on the second type P sensor, and (b) the target cell expresses sufficient levels of the type R marker to turn on the type R sensor.

104. The synthetic circuit of any one of claims 98 to 103, wherein: (a) the non-target cell expresses sufficient levels of the type P marker to turn on the second type P sensor, (b) the non-target cell does not express sufficient levels of the type R marker to turn on the type R sensor, or (c) both (a) and (b).

105. The synthetic circuit of any one of claims 86 to 104, wherein the payload sequence is a self-replicating RNA.

106. The synthetic circuit of any one of claims 86 to 105, wherein the regulator sequence is a non-replicating linear RNA.

107. The synthetic circuit of any one of claims 86 to 106, wherein the payload sequence is a circular RNA.

108. The synthetic circuit of any one of claims 86 to 107, wherein the regulator sequence is a circular RNA.

109. The synthetic circuit of any one of claims 86 to 104, wherein the payload sequence is a self-replicating RNA and the regulator sequence is a circular RNA.

110. The synthetic circuit of any one of claims 86 to 104, wherein the payload sequence is a self-replicating RNA and the regulator sequence is a non-replicating linear RNA.

111. The synthetic circuit of any one of claims 86 to 104, wherein the payload sequence is a circular RNA and the regulator sequence is a circular RNA.

112. The synthetic circuit of any one of claims 86 to 104, wherein the payload sequence is a circular RNA and the regulator sequence is a non-replicating linear RNA.

113. The synthetic circuit of any one of claims 86 to 112, wherein:
(a) the payload sequence comprises a plurality of the first type P sensor,
(b) the payload sequence comprises a plurality of the second type P sensor,
(c) the regulator sequence comprises a plurality of the type R sensor, or
(d) any combination of (a) to (c).

114. The synthetic circuit of any one of claims 86 to 113, wherein:
(a) the payload sequence comprises a spacer sequence (type P spacer),
(b) the payload sequence comprises a spacer sequence (type R spacer), or
(c) both (a) and (b).

115. The synthetic circuit of claim 114, wherein the type P spacer is positioned between the first type P sensor and the second type P sensor.

116. The synthetic circuit of claim 114 or 115, wherein the type P spacer is positioned between the payload coding sequence and (a) the first type P sensor, (b) the second type P sensor, or (c) both (a) and (b).

117. The synthetic circuit of any one of claims 114 to 116, wherein the type R spacer is positioned between the regulator coding sequence and the type R sensor.

118. The synthetic circuit of any one of claims 114 to 117, wherein the payload sequence comprises the plurality of the first type P sensor, wherein two or more of the first type sensors are separated by a type P spacer.

119. The synthetic circuit of any one of claims 114 to 118, wherein the payload sequence comprises the plurality of the second type P sensor, wherein two or more of the second type sensors are separated by a type P spacer.

120. The synthetic circuit of any one of claims 114 to 119, wherein the regulator sequence comprises the plurality of the type R sensor, wherein two or more of the type R sensors are separated by a type R spacer.

121. The synthetic circuit of any one of claims 114 to 120, wherein the type P spacer, type R spacer, or both are between about 1 to about 50 nucleotides in length.

122. The synthetic circuit of any one of claims 114 to 121, wherein the type P spacer, type R spacer, or both comprise, consist essentially of, or consist of the sequence tttcctttcccccttccctttttcctttcctttcctttcccccttccctt (SEQ ID NO: 1) or a fragment thereof.

123. The synthetic circuit of claim 122, wherein the type P spacer, type R spacer, or both comprise, consist essentially of, or consist of the sequence tttcctttcccccttccctt (SEQ ID NO: 2).

124. The synthetic circuit of any one of claims 114 to 121, wherein the type P spacer, type R spacer, or both comprise, consist essentially of, or consist of the sequence gcggccgctaaa (SEQ ID NO: 3) or a fragment thereof.

125. The synthetic circuit of any one of claims 87 to 124, wherein the type P marker, type R marker, or both comprise a microRNA.

126. The synthetic circuit of any one of claims 86 to 125, wherein the regulator comprises a RNA-binding protein, siRNA, aptamer, or combinations thereof.

127. The synthetic circuit of claim 126, wherein the RNA-binding protein comprises a ribonuclease.

128. The synthetic circuit of claim 127, wherein the ribonuclease comprises a Cas protein.

129. The synthetic circuit of claim 128, wherein the Cas protein comprises a Cas6 protein.

130. The synthetic circuit of any one of claims 1 to 129, wherein the payload comprises a therapeutic protein, reporter protein, immunomodulatory protein, chimeric antigen receptor (CAR), or combinations thereof.

131. The synthetic circuit of any one of claims 1 to 130, wherein the payload sequence comprises one or more elements that enhance the translation of the encoded protein as compared to the regulator sequence.

132. The synthetic circuit of claim 131, wherein the one or more elements comprise an aptamer for a translational initiation factor (e.g., eIF4G).

133. The synthetic circuit of any one of claims 1 to 132, which further comprises
(1) an Internal Ribosome Entry Site (IRES),
(3) a UTR,
(4) a sequence encoding a signal peptide,
(5) a translation initiation sequence,
(6) a polyA sequence,
(7) a sequence encoding a RNA binding protein,
(8) a sequence encoding a 2A ribosome skip peptide, or
(9) any combination of (1) to (8).

134. The synthetic circuit of any one of claims 1 to 133, which does not comprise any sequences derived from a non-human genome.

135. A vector comprising the synthetic circuit of any one of claims 1 to 134.

136. A nanoparticle comprising (i) the synthetic circuit of any one of claims 1 to 134, and (ii) one or more types of lipids and/or lipid like materials.

137. The nanoparticle of claim 136, wherein the one or more types of lipid comprise an ionizable lipid, cationic lipid, lipidoid, non-cationic helper lipid, phospholipid, sterol or other structural lipids, or combinations thereof

138. The nanoparticle of claim 137, wherein the ionizable lipid comprises ((4-hydroxybutyl)azanediyl)bis(hexane-6,1-diyl)bis(2-hexyldecanoate) (ALC-0315), heptadecan-9-yl 8-((2-hydroxyethyl)(6-oxo-6-(undecyloxy)hexyl)amino) octanoate (SM-102), heptadecan-9-yl 8-((2-hydroxyethyl)(8-(nonyloxy)-8-oxooctyl)amino)octanoate (Lipid 5), di((Z)-non-2-en-1-yl) 9-((4-(dimethylamino)butanoyl)oxy)heptadecanedioate (L319), 3-(didodecylamino)-N1,N1,4 tridodecyl-1-piperazineethanamine (KL10), Nl-[2 (didodecylamino)ethyl]-N1,N4,N4-tridodecyl 1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl-4-(dimethylamino)butanoate (DLin-MC3-DMA), 2,2-dilinoleyl-4-(2 dimethylaminoethyl)-[1,3]-dioxolane (DLin-KC2-DMA), 1,2-dioleyloxy-N,N-dimethylaminopropane (DODMA), 2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3f3)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), or combinations thereof.

139. The nanoparticle of claim 137, wherein the cationic lipid comprises 1,2-dioleoyl-3-trimethylammonium-propane (DOTAP), lipofectamine, N-[l-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTEVI), 2,3-dioleyloxy-N-[2(sperminecarboxamido)ethyl]-N,N-dimethyl-l-propanaminium trifluoroacetate (DOSPA), N,N-distearyl-N,N-dimethylammonium bromide (DDAB), N-(1,2-dimyristyloxyprop-3-yl)-N,N-dimethyl-N-hydroxy ethyl ammonium bromide (DMRIE), N-(1,2-dioleoyloxyprop-3-yl)-N,N-dimethyl-N-hydroxy ethyl ammonium bromide (DORIE), N,N-dioleyl-N,N-dimethylammonium chloride (DODAC), 1,2-dilauroyl-sn-glycero-3-ethylphosphocholine (DLePC), 1,2-distearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP), 1,2-distearoyl-sn-glycero-3-ethylphosphocholine (DSePC), 1,2-dipalmitoyl-sn-glycero-3-ethylphosphocholine (DPePC), 1,2-dimyristoyl-sn-glycero-3-ethylphosphocholine (DMePC), 1,2-dioleoyl-sn-glycero-3-ethylphosphocholine (DOePC), 1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), or any combination thereof.

140. The nanoparticle of claim 137, wherein the lipidoid comprises 1,1′-((2-(4-(2-((2-(bis(2-hydroxydodecyl) amino)ethyl) (2-hydroxydodecyl)amino)ethyl) piperazin-1-yl)ethyl)azanediyl) bis(dodecan-2-ol) (C12-200), 3,6-bis(4-(bis(2-hydroxydodecyl)amino)butyl)piperazine2,5-dione (cKK-E12), tetrakis(8-methylnonyl) 3,3′,3″,3′″-(((methylazanediyl) bis(propane-3,1 diyl))bis (azanetriyl))tetrapropionate (306Oi₁₀), G0-C14, 5A2-SC8, 3,6-bis(4-(bis((9Z,12Z)-2-hydroxyoctadeca9,12-dien-1-yl)amino)butyl)piperazine-2,5-dione (OF-02), (((3,6-dioxopiperazine-2,5-diyl)bis(butane-4,1-diyl))bis(azanetriyl))tetrakis(ethane2,1-diyl) (9Z,9′Z,9″Z,9′″Z,12Z,12′Z,12″Z,12′″Z)-tetrakis (octadeca-9,12-dienoate) (OF-Deg-Lin), (((3,6-dioxopiperazine-2,5-diyl)bis(butane-4,1-diyl)) bis(azanetriyl))tetrakis (butane-4,1-diyl) (9Z,9′Z,9″Z,9′″Z,12Z,12′Z,12″Z,12′″Z)-tetrakis (octadeca-9,12-dienoate) (OF-C4-Deg-Lin), Ni,N3,N5-tris(3-(didodecylamino)propyl)benzenel,3,5-tricarboxamide (TT3), Hexa(octan-3-yl) 9,9′,9″,9′″^{,9″} ″,9′″″-((((benzene-1,3,5-tricarbonyl)ris(azanediyl)) tris (propane-3,1-diyl))tris(azanetriyl))hexanonanoate (FTT5), PL-1, 98N12-5, ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18 (A2)), A12-Iso5-2DC18 (A12), or any combination thereof.

141. The nanoparticle of claim 140, wherein the lipidoid is TT3.

142. The nanoparticle of any one of claims 137 to 141, wherein the phospholipid is selected from the group consisting of 1,2-dilinoleoyl-sn-glycero-3 phosphocholine (DLPC), 1,2-dimyristoyl-sn-glycerol-phosphocholine (DMPC), 1,2-dioleoyl-sn glycerol-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and any combinations thereof.

143. The nanoparticle of any one of claims 137 to 142, wherein the phospholipid is selected from the group consisting of 1-myristoyl-2-palmitoyl-sn-glycero-3-phosphocholine (14:0-16:0 PC, MPPC), 1-myristoyl-2 stearoyl-sn-glycero-3-phosphocholine (14:0-18:0 PC, MSPC), 1-palmitoyl 2-acetyl-sn-glycero-3-phosphocholine (16:0-02:0 PC), 1-palmitoyl-2-myristoyl-sn-glycero-3-phosphocholine (16:0-14:0 PC, PMPC), 1-palmitoyl-2-stearoyl-sn-glycero-3-phosphocholine (16:0-18:0 PC, PSPC), 1-palmitoyl-2-oleoyl-sn-glycero-3-phosphocholine (16:0-18:1 PC, POPC), 1-palmitoyl-2-linoleoyl-sn-glycero-3-phosphocholine (16:0-18:2 PC, PLPC), 1-palmitoyl-2-arachidonoyl-sn-glycero-3-phosphocholine (16:0-20:4 PC), 1-palmitoyl-2-docosahexaenoyl-sn-glycero-3-phosphocholine (14:0-22:6 PC), 1-stearoyl-2-myristoyl-sn-glycero-3-phosphocholine (18:0-14:0 PC, SMPC), 1-stearoyl-2-palmitoyl-sn-glycero-3-phosphocholine (18:0-16:0 PC, SPPC), 1-stearoyl-2-oleoyl-sn-glycero-3-phosphocholine (18:0-18:1 PC, SOPC), 1-stearoyl-2-linoleoyl-sn-glycero-3-phosphocholine (18:0-18:2 PC), 1-stearoyl-2-arachidonoyl-sn-glycero-3-phosphocholine (18:0-20:4 PC), 1-stearoyl-2-docosahexaenoyl-sn-glycero-3-phosphocholine (18:0-22:6 PC), 1-oleoyl-2-myristoyl-sn-glycero-3-phosphocholine (18: 1-14:0 PC, OMPC), 1-oleoyl-2-palmitoyl-sn-glycero-3-phosphocholine (18: 1-16:0 PC, OPPC), 1-oleoyl-2-stearoyl-sn-glycero-3-phosphocholine (18: 1-18:0 PC, OSPC), 1-palmitoyl-2-oleoyl-sn-glycero-3-phosphoethanolamine (16:0-18:1 PE, POPE), 1-palmitoyl-2-linoleoyl-sn-glycero-3-phosphoethanolamine (16:0-18:2 PE), 1-palmitoyl-2-arachidonoyl-sn-glycero-3-phosphoethanolamine (16:0-20:4 PE), 1-palmitoyl-2-docosahexaenoyl-sn-glycero-3-phosphoethanolamine (16:0-22:6 PE), 1-stearoyl-2-oleoyl-sn-glycero-3-phosphoethanolamine (18:0-18:1 PE), 1-stearoyl-2-linoleoyl-sn-glycero-3-phosphoethanolamine (18:0-18:2 PE), 1-stearoyl-2-arachidonoyl-sn-glycero-3-phosphoethanolamine (18:0-20:4 PE), 1-stearoyl-2-docosahexaenoyl-sn-glycero-3-phosphoethanolamine (18:0-22:6 PE), 1-oleoyl-2-cholesterylhemisuccinoyl-sn-glycero-3-phosphocholine (OChemsPC), and any combination thereof.

144. The nanoparticle of any one of claims 137 to 143, wherein the sterol comprises a cholesterol, fecosterol, sitosterol, ergosterol, campesterol, stigmasterol, brassicasterol, tomatidine, tomatine, ursolic acid, alpha-tocopherol, and combinations thereof

145. The nanoparticle of any one of claims 136 to 144, wherein the one or more types of lipids and/or lipid like materials are pegylated.

146. The nanoparticle of any one of claims 136 to 145, which further comprises a targeting ligand.

147. The nanoparticle of any one of claims 136 to 146, wherein the one or more types of lipids and/or lipid like materials comprise a molar ratio of about 10-50% ionizable lipid (e.g., cationic lipid).

148. The nanoparticle of any one of claims 136 to 147, wherein the one or more types of lipids and/or lipid like materials comprise a molar ratio of about 10-40% phospholipid.

149. The nanoparticle of any one of claims 136 to 148, wherein the one or more types of lipids and/or lipid like materials comprise a molar ratio of about 20-50% sterol (e.g., cholesterol).

150. The nanoparticle of any one of claims 136 to 149, wherein the one or more types of lipids and/or lipid like materials comprise a molar ratio of about 0-10% pegylated lipid.

151. A pharmaceutical composition comprising the synthetic circuit of any one of claims 1 to 134, the vector of claim 135, or the nanoparticle of any one of claims 136 to 150, and a pharmaceutically acceptable carrier.

152. The pharmaceutical composition of claim 151, which is formulated for intratumoral, intrathecal, intramuscular, intravenous, subcutaneous, inhalation, intradermal, intralymphatic, intraocular, intraperitoneal, intrapleural, intraspinal, intravascular, nasal, percutaneous, sublingual, submucosal, transdermal, or transmucosal administration.

153. A cell comprising the synthetic circuit of any one of claims 1 to 134, the vector of claim 135, or the nanoparticle of any one of claims 136 to 150.

154. A cell comprising a payload expressed by the synthetic circuit of any one of claims 1 to 134, the vector of claim 135, or the nanoparticle of any one of claims 136 to 150.

155. A method of treating a disease or disorder in a subject in need thereof, comprising administering to the subject the synthetic circuit of any one of claims 1 to 134, the vector of claim 135, the nanoparticle of any one of claims 136 to 150, the pharmaceutical composition of claim 151 or 152, or the cell of claim 153 or 154.

156. The method of claim 155, wherein the subject receives multiple administrations of the synthetic circuit, the vector, the nanoparticle, or the pharmaceutical composition.

157. A method of inducing the expression of a payload in a cell, comprising contacting the cell with the synthetic circuit of any one of claims 1 to 134, the vector of claim 135, the nanoparticle of any one of claims 136 to 150, the pharmaceutical compostion of claim 151 or 152, or the cell of claim 153 or 154, wherein the payload is expressed in the cell if the regulator is not expressed in the cell.

158. The method of claim 155 or 156, wherein the disease or disorder comprises a cancer.

159. The method of any one of claims 155, 156, and 158, wherein the payload comprises a chimeric antigen receptor (CAR), T cell receptor (TCR) or TCR mimic.

160. A method of generating immune cells expressing a chimeric antigen receptor (CAR), T cell receptor (TCR) or TCR mimic in vivo in a subject in need thereof comprising administering the synthetic circuit of any one of claims 1 to 134, the vector of claim 135, the nanoparticle of any one of claims 136 to 150, or the pharmaceutical compostion of claim 151 or 152, wherein the synthetic circuit expresses the CAR, TCR, or TCR mimic as a payload.

161. A method of treating cancer in a subject in need thereof by in situ generated immune cells expressing a chimeric antigen receptor (CAR), T cell receptor (TCR) or TCR mimic, comprising administering the synthetic circuit of any one of claims 1 to 134, the vector of claim 135, the nanoparticle of any one of claims 136 to 150, or the pharmaceutical compostion of claim 151 or 152, wherein the synthetic circuit expresses the CAR, TCR, or TCR mimic as a payload.

162. The method of any one of claims 159 to 161, wherein the CAR targets CD19, TRAC, TCRβ, BCMA, CLL-1, CS1, CD38, CD19, TSHR, CD123, CD22, CD30, CD70, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin BI, MYCN, RhoC, TRP-2, CYPlB1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLECl2A, BST2, EMR2, LY75, GPC3, FCRL5, IGLL1, CD2, CD3F, CD4, CD5, CD7, the extracellular portion of the APRIL protein, or any combinations thereof.

163. The method of any one of claims 159 to 161, wherein the TCR targets AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-l Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTl, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p5³, p⁵³ mutant, prostein, surviving, telomerase, PCTA-1/Galectin 8, MelanA/MARTl, Ras mutant, hTERT, sarcoma translocation breakpoints, ML-IAP, ERG (TMPRSS2 ETS fusion gene), NA17, PAX3, androgen receptor, cyclin BI, MYCN, RhoC, TRP-2, CYPlB1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3F, CD4, CD5, CD7, the extracellular portion of the APRIL protein, or any combinations thereof.


SEQUENCE LISTING (10 sequences)

SEQ ID NO: 1 - RNA, 50 nt, synthetic construct
  source 1..50: mol_type = other RNA; note = Spacer
tttcctttcccccttccctttttcctttcctttcctttcccccttccctt

SEQ ID NO: 2 - RNA, 20 nt, synthetic construct
  source 1..20: mol_type = other RNA; note = Spacer
tttcctttcccccttccctt

SEQ ID NO: 3 - RNA, 12 nt, synthetic construct
  source 1..12: mol_type = other RNA; note = Spacer
gcggccgctaaa

SEQ ID NO: 4-5 - skipped (2 entries)

SEQ ID NO: 6 - protein, 349 aa, synthetic construct
  source 1..349: note = WT PUF RBD
GRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 7 - protein, 286 aa, synthetic construct
  source 1..286: note = cNOT7
MPAATVDHSQRICEVWACNLDEEMKKIRQVIRKYNYVAMDTEFPGVVARPIGEFRSNADYQYQLLRCNVDLLKIIQLGLTFMNEQGEYPPGTSTWQFNFKFNLTEDMYAQDSIELLTTSGIQFKKHEEEGIETQYFAELLMTSGVVLCEGVKWLSFHSGYDFGYLIKILTNSNLPEEELDFFEILRLFFPVIYDVKYLMKSCKNLKGGLQEVAEQLELERIGPQHQAGSDSLLTGMAFFKMREMFFEDHIDDAKYCGHLYGLGSGSSYVQNGTGNAYEEEANKQSV

SEQ ID NO: 8 - protein, 326 aa, synthetic construct
  source 1..326: note = TTP
MDLTAIYESLLSLSPDVPVPSDHGGTESSPGWGSSGPWSLSPSDSSPSGVTSRLPGRSTSLVEGRSCGWVPPPPGFAPLAPRLGPELSPSPTSPTATSTTPSRYKTELCRTFSESGRCRYGAKCQFAHGLGELRQANRHPKYKTELRHKFYLQGRCPYGSRCHFIHNPSEDLAAPGHPPVLRQSISFSGLPSGRRTSPPPPGLAGPSLSSSSFSPSSSPPPPGDLPLSPSAFSAAPGTPLARRDPTPVCCPSCRRATPISVWGPLGGLVRTPSVQSLGSDPDEYASSGSSLGGSDSPVFEAGVFAPPQPVAAPRRLPIFNRISVSE

SEQ ID NO: 9 - protein, 483 aa, synthetic construct
  source 1..483: note = DDX6
MSTARTENPVIMGLSSQNGQLRGPVKPTGGPGGGGTQTQQQMNQLKNTNTINNGTQQQAQSMTTTIKPGDDWKKTLKLPPKDLRIKTSDVTSTKGNEFEDYCLKRELLMGIFEMGWEKPSPIQEESIPIALSGRDILARAKNGTGKSGAYLIPLLERLDLKKDNIQAMVIVPTRELALQVSQICIQVSKHMGGAKVMATTGGTNLRDDIMRLDDTVHVVIATPGRILDLIKKGVAKVDHVQMIVLDEADKLLSQDFVQIMEDIILTLPKNRQILLYSATFPLSVQKFMNSHLQKPYEINLMEELTLKGVTQYYAYVTERQKVHCLNTLFSRLQINQSIIFCNSSQRVELLAKKISQLGYSCFYIHAKMRQEHRNRVFHDFRNGLCRNLVCTDLFTRGIDIQAVNVVINFDFPKLAETYLHRIGRSGRFGHLGLAINLITYDDRFNLKSIEEQLGTEIKPIPSNIDKSLYVAEYHSEPVEDEKP

SEQ ID NO: 10 - protein, 190 aa, synthetic construct
  source 1..190: note = MCPIP1 PIN; note = MCPIP1 PIN
MGGGTPKAPNLEPPLPEEEKEGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAYESDGIVVSNDTYRDLQGERQEWKRFIEERLLMYSFVNDKFMPPDDPLGRHGPSLDNFLRKKPLTLE